(19) United States
(12) Patent Application Publication
(10) Pub. No.: US 2019/0151437 A1
(43) Pub. Date: May 23, 2019

(54) COMBINATION PRIME: BOOST THERAPY

(71) Applicant: TURNSTONE LIMITED PARTNERSHIP, Toronto, Ontario (CA)

(72) Inventors: Brian Lichty, Brantford (CA); Kyle Stephenson, Smithville (CA); Matthew Atherton, Wynnewood, PA (US)

(21) Appl. No.: 16/300,014

(22) PCT Filed: May 9, 2017
(86) PCT No.: PCT/IB2017/000622

§ 371 Date: Nov. 8, 2018

Related U.S. Application Data
(60) Provisional application No. 62/402,670, filed on Sep. 30, 2016.
(60) Provisional application No. 62/333,685, filed on May 9, 2016.

(51) Int. Cl. A61K 39/12; A61K 39/00; A61P 35/00; A61K 35/766; C07K 14/025

(52) U.S. Cl. CPC A61K 39/12; A61K 39/0011; A61K 2039/54; A61K 35/766; C07K 14/025; A61P 35/00

(57) ABSTRACT

A combination prime:boost therapy is described herein. The combination therapy is for use in inducing an immune response in a mammal. The combination includes: an adenovirus that is capable of expressing an antigenic protein, and that is formulated to generate an immunity to the protein in the mammal; and a Maraba MG1 virus that is capable of expressing an antigenic protein, and that is formulated to induce the immune response in the mammal. The antigenic proteins are both based on the same tumour associated antigen, but need not be identical. An adenovirus, methods of treatment, and uses are also described.

### CROSS REFERENCE TO RELATED APPLICATIONS

This application claims the benefit of priority of U.S. Provisional Patent Application No. 62/333,685 filed May 9, 2016, and U.S. Provisional Patent Application No. 62/402,670 filed Sep. 30, 2016, which are hereby incorporated by reference.

### FIELD

The present disclosure relates to oncolytic viruses for inducing an immune response.

### BACKGROUND

Oncolytic viruses (OVs) specifically infect, replicate in and kill malignant cells, leaving normal tissues unaffected. Several OVs have reached advanced stages of clinical evaluation for the treatment of various neoplasms (Russell S J. et al., (2012) Nat Biotechnol 30:658-670). Once approved, such viral agents could substitute or combine with standard cancer therapies and allow for reduced toxicity and improved therapeutic efficacy.

In addition to the vesicular stomatitis virus (VSV) (Stojdl D F. et al., (2000) Nat Med 6:821-825; Stojdl D F. et al., (2003) Cancer Cell 4:263-275), other rhabdoviruses displaying oncolytic activity have been described recently (Brun J. et al., (2010) Mol Ther 18:1440-1449; Mahoney D J. et al., (2011) Cancer Cell 20:443-456). Among them, the non-VSV Maraba virus showed the broadest oncotropism in vitro (WO 2009/016433). A mutant Maraba virus with improved tumor selectivity and reduced virulence in normal cells was engineered. The attenuated strain is a double mutant strain containing both G protein (Q242R) and M protein (L123W) mutations. In vivo, this attenuated strain, called MG1 or Maraba MG1, demonstrated potent anti-tumor activity in xenograft and syngeneic tumor models in mice, with superior therapeutic efficacy than the attenuated VSV, VSVΔM51 (WO 2011/070440).

Data accumulated over the past several years has revealed that anti-tumor efficacy of oncolytic viruses not only depends on their direct oncolysis but may also depend on their ability to stimulate anti-tumor immunity (Bridle B W. et al., (2010) Mol Ther 184:4269-4275). This immune-mediated tumor control seems to play a critical role in the overall efficacy of OV therapy. Indeed, tumor-specific adaptive immune cells can patrol the tissues and destroy tumor cells that have been missed by the OV. Moreover, their memory compartment can prevent tumor recurrence.

Various strategies have been developed to improve OV-induced anti-tumor immunity (Poi J. et al., (2012) Virus Adaptation and Treatment 4:1-21). Some groups have genetically engineered OV expressing immunomostimulatory cytokines. A herpes simplex and a vaccinia virus expressing Granulocyte-Macrophage Colony-Stimulating Factor (GM-CSF) have respectively reached phase III and IIB of the clinical evaluation for cancer therapy while a VSV expressing IFN-β has just entered phase I.

Another strategy, defined as an oncolytic vaccine, consists of expressing a tumor antigen from the OV (Russell S J. et al., (2012) Nat Biotechnol 30:658-670). Previously, it has been demonstrated that VSV could also be used as a cancer vaccine vector (Bridle B W. et al., (2010) Mol Ther 184:4269-4275). When applied in a heterologous prime:boost setting to treat a murine melanoma model, a VSV-human dopachrome tautomerase (hDCT) oncolytic vaccine not only induced an increased tumor-specific immunity to DOT but also a concomitant reduction in antiviral adaptive immunity. As a result, the therapeutic efficacy was dramatically improved with an increase of both median and long term survivals (WO 2010/105347). Although VSV was shown to be effective using hDCT as a tumor associated antigen, there is no way to predict what tumor associated antigens will be effective in a heterologous prime:boost setting.

Three specific prime:boost combination therapies are disclosed in PCT Application No. PCT/CA2014/050118. The combination therapies include a lentivirus that encodes as an antigen: a Human Papilloma Virus (HPV) E6/E7 fusion protein, human Six-Transmembrane Epithelial Antigen of the Prostate (huSTEAP) protein, or Cancer Testis Antigen 1; and a Maraba MG1 virus that encodes the same antigen. PCT Application No. PCT/CA2014/050118 also discloses a prime:boost combination therapy using an adenovirus that encodes MAGEA3 as an antigen, and a Maraba MG1 virus that encodes the same antigen.

### SUMMARY

The following summary is intended to introduce the reader to one or more inventions described herein but not to define any one of them.

It is an object of the present disclosure to obviate or mitigate at least one disadvantage of previous anti-cancer vaccines.

The authors of the present disclosure have identified a combination prime:boost therapy that induces an immune response in a mammal. In contrast to the prime:boost combination therapy disclosed in PCT Application No. PCT/CA2014/050118, discussed above, which uses a lentivirus as the “prime”, the combination prime:boost therapy according to the present disclosure uses a recombinant adenovirus expressing the antigen as the priming virus. A recombinant Maraba MG1 virus expressing the antigen is used as the boosting virus. Exemplary combination therapies according to the present disclosure use HPV E6/E7 or STEAP as the antigen.

The results discussed herein show that a recombinant adenovirus provides at least one advantage over the recombinant lentivirus of the '118 POT application. These results are unexpected and not predictable because there is no way to predict if or how efficacy will be affected if the priming virus is changed. One would not have be able to predict which, if any, priming virus would provide a beneficial effect on the immune response in a prime:boost combination therapy.

In one aspect, there is provided a combination prime:boost therapy for use in inducing an immune response in a mammal. The combination therapy includes: an adenovirus that (a) expresses an antigenic protein and (b) is formulated to generate an immunity to the protein in the mammal. The combination therapy also includes a Maraba MG1 virus that (a) expresses an antigenic protein and (b) is formulated to induce the immune response in the mammal. The antigenic proteins expressed by the adenovirus and the Maraba MG1 virus are based on the same tumor associated antigen, but do not need to be identical in sequence.

In some examples of the combination therapy, the antigenic protein is a Human Papilloma Virus E6/E7 fusion protein. In other examples of the combination therapy, the antigen protein is a huSTEAP protein.

In other aspects, the present disclosure provides for uses of the combination of viruses, and methods of using the combination of viruses. The uses and methods may relate to: treatment or prevention of an HPV-derived cancer, such as a cancer caused by HPV16 or HPV18; increasing an immune response against E6 and/or E7 proteins; or combinations thereof.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is an illustration of the sequence of an exemplary HPV E6/E7 fusion protein that may be expressed by an adenovirus and a Maraba MG1 virus and used in an combination prime:boost therapy according to the present disclosure.

FIG. 2 is a graph illustrating the interaction of the exemplary HPV E6/E7 fusion with p53.

FIG. 3 is a graph illustrating the interaction of the exemplary HPV E6/E7 fusion with pRb.

FIGS. 4A-4D is a set of graphs illustrating the stimulation of an immune response against an E6 or an E7 epitope, as measured by the percentage of CD8+ T cells secreting interferon-γ (IFNγ), of a combination prime:boost therapy according to the present disclosure, and of control therapies.

FIGS. 5A and 5B are graphs illustrating the number of CD8+ T cells, and the total number of E7 specific CD8+ T cells, generated after a combination prime:boost therapy according to the present disclosure, versus after the prime only.

FIG. 6 is a graph illustrating the immune response against an E7 epitope, as measured by percentage of CD8+ T cells secreting interferon-γ (IFNγ) over time, of a combination prime:boost therapy according to the present disclosure, and of control therapies.

FIGS. 7A-7D are graphs illustrating the quality of the generated T cell response, as measured by double positive (IFNγ and TNFα) or triple positive (IFNγ, TNFα and IL-2) CD8+ T cells found in the circulatory and splenic pools.

FIGS. 8A and 8B are graphs illustrating expansion of E6 and E7 specific T cells in tumour bearing mice after an exemplary combination prime:boost therapy according to the present disclosure, as measured the percentage of CD8+ T cells secreting interferon-γ (IFNγ) by intracellular staining.

FIG. 9 is a graph illustrating percent survival vs. time of mice treated with a combination prime:boost therapy according to the present disclosure, and control treatments.

FIG. 10 is a graph illustrating percent survival vs. time of mice treated with a curative combination prime:boost therapy according to the present disclosure, with depletion of CD8+ T cells at different times in the treatment.

FIGS. 11A and 11B are graphs illustrating persistence in the blood and spleen of E7 specific CD8+ T cells, at 62 and 117 days after boosting using an exemplary combination prime:boost therapy according to the present disclosure.

FIG. 12 is an illustration of a treatment schema for the test discussed in Example 7.

FIG. 13 is a graph illustrating the ex vivo peptide re-stimulation responses to specific peptide antigens after priming tumour-free mice with Ad-huSTEAP.

FIG. 14 is a graph illustrating the ex vivo peptide re-stimulation responses to specific peptide antigens after priming tumour-free mice with Ad-huSTEAP and subsequent boosting with MG1-huSTEAP.

FIG. 15 is an illustration of a treatment schema for the test discussed in Example 8.

FIG. 16 is a graph illustrating the ex vivo peptide re-stimulation responses to specific peptide antigens after priming mice engrafed with TrampC2 cells with Ad-huSTEAP.

FIG. 17 is a graph illustrating the ex vivo peptide re-stimulation responses to specific peptide antigens after priming mice engrafed with TrampC2 cells with Ad-huSTEAP and subsequent boosting with MG1-huSTEAP.

FIG. 18 is an illustration of TrampC2 tumour growth by volume in the three mice groups.

FIG. 19 is an illustration of mice survival over time in the three mice groups.

### DETAILED DESCRIPTION

The present disclosure provides a combination prime:boost therapy for use in inducing an immune response in a mammal. Prime:boost immunizations can be given with unmatched vaccine delivery methods while using the same antigen, in a ‘heterologous’ prime:boost format; or with matched vaccine delivery methods, in a ‘homologous’ prime:boost. Heterologous prime:boost methods are preferable when using vectored vaccine platforms as homologous vaccination would lead to boosting of responses to both the vector and the transgene in the secondary response. In contrast, a heterologous system focuses the secondary response (that is, the boosted response) on the antigen as responses against the first and the second vector are primary responses, and are therefore much less robust.

Generally, a combination prime:boost therapy of the present disclosure includes: (1) an adenovirus that is capable of expressing an antigenic protein and that is formulated to generate an immunity to the protein in the mammal; and (2) a Maraba MG1 virus that is capable of expressing an antigenic protein and that is formulated to induce the immune response in the mammal.

The antigenic protein expressed by the adenovirus and the antigenic protein expressed by the Maraba MG1 virus may be identical, or different. If different, the antigenic proteins are sufficiently similar that the immune response to the antigenic protein expressed by the Maraba MG1 virus is increased in comparison to an immune response induced in the absence of a priming virus.

In some exemplary combination therapies according to the present disclosure, the therapy may be used to activate the patient's immune system to kill tumour cells with reduced toxicity to normal tissues, for example by activating antibodies and/or lymphocytes against a tumor associated antigen on the tumour. In particular examples, the therapy may display both oncolytic activity and an ability to boost adaptive cell immunity.

In one aspect, the combination prime:boost therapy of the present disclosure includes: (1) an adenovirus that is capable of expressing a Human Papilloma Virus E6/E7 fusion protein as an antigenic protein and that is formulated to generate an immunity to the protein in the mammal; and (2) a Maraba MG1 virus that is capable of expressing a Human Papilloma Virus E6/E7 fusion protein as an antigenic protein and that is formulated to induce the immune response in the mammal. The antigenic protein expressed by the adenovirus and the antigenic protein expressed by the Maraba MG1 virus may be identical, or different.

In another aspect, the combination prime:boost therapy of the present disclosure includes: (1) an adenovirus that is capable of expressing a human Six-Transmembrane Epithelial Antigen of the Prostate (huSTEAP) protein as an antigenic protein and that is formulated to generate an immunity to the protein in the mammal; and (2) a Maraba MG1 virus that is capable of expressing a huSTEAP protein as an antigenic protein and that is formulated to induce the immune response in the mammal. The antigenic protein expressed by the adenovirus and the antigenic protein expressed by the Maraba MG1 virus may be identical, or different.

In the context of the present disclosure, the terms “priming adenovirus” and “boosting maraba virus” should be understood to refer to an adenovirus that is capable of expressing an antigenic protein, and a Maraba MG1 virus that is capable of expressing an antigenic protein, respectively. The terms “Ad-E6E7”, “Adenovirus E6E7”, and “Adenovirus encoding HPV E6/E7 protein” should all be understood to refer to an adenovirus that is capable of expressing a Human Papilloma Virus E6/E7 fusion protein as an antigenic protein; and the terms “MG1-E6E7”, “Maraba MG1 E6E7”, and “Maraba MG1 virus encoding HPV E6/E7 protein” should all be understood to refer to a Maraba MG1 virus that is capable of expressing a Human Papilloma Virus E6/E7 fusion protein. Similarly, the terms “Ad-huSTEAP”, “Adenovirus huSTEAP”, “Adenovirus encoding huSTEAP protein” and “priming adenovirus” should all be understood to refer to an adenovirus that is capable of expressing a huSTEAP protein as an antigenic protein; and the terms “MG1-huSTEAP”, “Maraba MG1 huSTEAP”, and “Maraba MG1 virus encoding huSTEAP” should all be understood to refer to a Maraba MG1 virus that is capable of expressing a huSTEAP protein.

Human Papilloma Virus E6/E7 fusion protein is one example of an antigenic protein that may be used in therapies and methods according to the present disclosure. HPV E6/E7 includes sequences corresponding to the E6 and E7 transforming proteins of both the HPV16 and HPV18 serotypes, resulting in a fusion protein that includes HPV16 E6, HPV18 E6, HPV16 E7 and HPV18 E7 protein domains. The four protein domains are linked by proteasomally degradable linkers that result in the separate HPV16 E6, HPV18 E6, HPV16 E7 and HPV18 E7 proteins once the fusion protein is in the proteasome. The proteasomally degradeable linkers in a fusion protein may be the same or different. The terms “HPV E6/E7 protein”, “HPV E6/E7 fusion protein”, and “therapeutic E6E7 construct” should all be understood to be synonymous with “Human Papilloma Virus E6/E7 fusion protein”.

One example of a Human Papilloma Virus E6/E7 fusion protein according to the present disclosure has an amino acid sequence according to SEQ ID NO: 1. In SEQ ID NO: 1, the proteasomally degradable linkers have the sequence GGGGGAAY (SEQ ID NO: 2). Other proteasomally degradable linkers could alternatively be used.

To generate an HPV E6/E7 fusion protein according to SEQ ID NO: 1, the Maraba MG1 virus genome may include a reverse complement and RNA version of a nucleotide sequence of SEQ ID NO: 3. In specific examples, the Maraba MG1 virus genome may include a nucleotide sequence that is the reverse complement and RNA version of SEQ ID NO: 4.

To generate an HPV E6/E7 fusion protein according to SEQ ID NO: 1, the adenovirus may include a transgene comprising a nucleotide sequence of SEQ ID NO: 3. The transgene may additionally include a promoter, such as murine cytomegalovirus (MCMV) IE promoter, preceding the HPV E6E7 encoding region. The transgene may additionally include, preferably in combination with the promoter, a region encoding a SV40 polyadenylation signal sequence after the HPV E6E7 encoding region.

Because the HPV16 E6, HPV18 E6, HPV16 E7 and HPV18 E7 proteins are separated from each other in the proteasome (due to the presence of the proteasomally degrabable linkers in the fusion protein), a Human Papilloma Virus E6/E7 fusion protein according to the present disclosure may have a sequence where the protein domains are rearranged in a different order than they are in SEQ ID NO: 1 and still provide the HPV16 E6, HPV18 E6, HPV16 E7 and HPV18 E7 proteins.

If the sequence of the HPV16 E6, HPV18 E6, HPV16 E7 and HPV18 E7 domains in SEQ ID NO: 1 corresponded to A, B, C, D, respectively, a Human Papilloma Virus E6/E7 fusion protein according to the present disclosure could have a sequence where the four domains were rearranged in any of the other 23 possible permutations, for example: ABDC, ACBD, ACDB, ADBC, ADCB, BACD, BACD, BADC, CABD, CADB, DACB, DCAB, DCBA, etc. Four specific examples of such rearrangements of SEQ ID NO: 1 are shown in SEQ ID NOs: 5-8, but it should be understood that the present disclosure also contemplates the other nineteen permutations, and that sequences of such permutations are readily derivable from the protein domains and the linkers disclosed in SEQ ID NOs: 1 and 5-8.

Although HPV E6/E7 fusion proteins according to the present disclosure may be formed from wild type sequences of the HPV16 E6, HPV18 E6, HPV16 E7 and HPV18 E7 proteins, it is desirable to modify the wild type sequences to prevent the formation of zinc fingers. If cells transduced with adenovirus HPV E6/E7 were to undergo an integration event with an E6E7 transgene that encoded a fusion protein that produced E6 and E7 proteins that could not form zinc fingers, the proteins produced would be unable to interfere with the functions of p53 or retinoblastoma, thereby reducing the possibility of a de novo neoplasm from forming.

In preferred examples, the sequences of one or more of the wild type HPV16 E6, HPV18 E6, HPV16 E7 and HPV18 E7 proteins may be modified to abrogate the ability of one or more CXXC motifs to form zinc fingers. The sequences of one or more of the wild type HPV16 E7 and HPV18 E7 proteins may additionally, or alternatively, be modified to abrogate the ability of a LXCXE sequence motif to bind to Retinoblastoma (Rb) protein. Preventing the formation of zinc fingers may be achieved, for example, by deleting one or both of the cysteines in a CXXC motif. Preventing the bind to Rb protein may be achieved, for example, by deleting one or more of the amino acids in a LXCXE sequence motif, such as deleting the CXE amino acids. In an alternative to deleting amino acids, replacing one or more of the amino acids in either the CXXC or LXCXE motifs with other amino acids, such as alanine, may prevent binding. Preferably, all four of the protein sequences are modified to prevent the separated E6 and E7 proteins from forming zinc fingers and/or from binding to Rb protein.

The sequences of HPV16 E6, HPV18 E6, HPV16 E7 and HPV18 E7 proteins which may be used in a Human Papilloma Virus E6/E7 fusion protein are shown in SEQ ID NOs: 9-12.
In SEQ ID NOs: 9 and 10, each Xaa is independently: absent, cysteine, or a non-cystine amino acid. When the identified variable Xaa's residules are cysteines, the sequences correspond to the wild type sequences of HPV16 E6 and HPV18 E6, respectively.
In SEQ ID NO: 11, the Xaa at position 24 is either absent, cysteine, or a non-cysteine amino acid; the Xaa at position 25 is either absent, tyrosine, or a non-tyrosine amino acid; the Xaa at position 26 is either absent, glutamic acid, or a non-glutamic acid amino acid; and the Xaa's at positions 91 and 94 are, independently; absent, cysteine, or a non-cysteine amino acid. The sequence of SEQ ID NO: 11 corresponds to the wild type sequence of HPV16 E7 when the Xaa's at positions 24-26 are cysteine-tyrosine-glutamic acid and the Xaa's at positions 91 and 94 are cysteines.
In SEQ ID NO: 12, the Xaa at position 27 is either absent, cysteine, or a non-cysteine amino acid; the Xaa at position 28 is either absent, histidine, or a non-histidine amino acid; the Xaa at position 29 is either absent, glutamic acid, or a non-glutamic acid amino acid; and the Xaa's at positions 98 and 101 are, independently: absent, cysteine, or a non-cysteine amino acid. The sequence of SEQ ID NO: 12 corresponds to the wild type sequence of HPV18 E7 when the Xaa's at positions 27-29 are cysteine-histidine-glutamic acid and the Xaa's at positions 98 and 101 are cysteines.

A Human Papilloma Virus E6/E7 fusion protein according to the present disclosure may be defined as a fusion protein that includes, in any order, four protein domains having sequences according to SEQ ID NOs: 9, 10, 11 and 12, where the protein domains are linked by proteasomally degradable linkers, which may be the same or different.

Preferably, at least one Xaa in each of SEQ ID NOs: 9, 10, 11 and 12 is absent. More preferably, sufficient Xaa's are absent to reduce zinc finger formation in the separated proteins that are generated in the proteasome. In some preferred examples, the Xaa's in the first CXXC motifs of both of SEQ ID NOs: 9 and 10 are absent.

The proteasomally degradable linkers are preferably amino acid linkers having the sequence: GGGGGAAY.

It should be understood that the definition above corresponds to SEQ ID NO: 1 when:
the protein domains are arranged in the order: SEQ ID NO: 9 then SEQ ID NO: 10 then SEQ ID NO: 11 then SEQ ID NO: 12;
the protein domains are linked together by linkers having the sequence GGGGGAAY;
the Xaa's at positions 70, 73, 110 and 113 in SEQ ID NO: 9 are absent;
the Xaa's at positions 65, 68, 105 and 108 in SEQ ID NO: 10 are absent;
the Xaa's at positions 24-26 and 91 in SEQ ID NO: 11 are absent; and
the Xaa's at positions 27-29 and 98 in SEQ ID NO: 12 are absent.

It should be understood that all the variables discussed above with respect to the HPV E6/E7 fusion protein (such as: the order of the protein domains, the sequence of the proteasomally degradable linkers, whether the proteasomally degradable linkers are the same or different, and whether the wildtype sequences are modified to prevent the formation of zinc fingers) can be used alone or in combination to generate protein sequences that would be considered an HPV E6/E7 fusion protein according to the present disclosure.

Another example of an antigenic protein that may be used in therapies and methods according to the present disclosure is Six-Transmembrane Epithelial Antigen of the Prostate (STEAP) protein. Human STEAP (huSTEAP) is overexpressed in prostate cancer and up-regulated in multiple cancer cell lines, including pancreas, colon, breast, testicular, cervical, bladder, ovarian, acute lyphocytic leukemia, and Ewing sarcoma (Hubert R S et al., (1999) Proc Natl Acad Sci 96: 14523-14528).

The STEAP gene encodes a protein with six potential membrane-spanning regions flanked by hydrophilic amino- and carboxyl-terminal domains. The huSTEAP protein was used by the authors of the present disclosure as the antigenic protein in both the priming adenovirus and the Maraba MG1 virus. In the present disclosure, the authors tested a codon-optimized sequence for expression in human and mouse that gives rise to a 341 amino acid protein (SEQ ID NO: 13). A negative sense RNA virus that expresses the protein of SEQ ID NO: 13 may include a reverse complement and RNA version of a polynucleotide of SEQ ID NO: 14. A DNA virus that expresses the protein of SEQ ID NO: 13 may include a sequence that is SEQ ID NO: 14.

Maraba MG1 was engineered to contain human Six-Transmembrane Epithelial Antigen of the Prostate transgene inserted between the G and L viral genes of the MG1 double mutant of Maraba virus (Brun J. et al., (2010) Mol Ther 8: 1440-1449). The transgene sequence was codon optimized for expression in mammalian cells. The resulting Maraba MG1 containing the huSTEAP protein is designated as “Maraba-MG1-huSTEAP” or “MG1-huSTEAP”. A modified Maraba MG1 backbone was used to facilitate cloning. A silent mutation was introduced into the L gene of the Maraba MG1 genome backbone to remove one of the MluI sites. The second MluI site was replaced with a BsiWI site at the cloning region between G and L. These modifications to the Maraba MG1 genome backbone allowed for a more direct cloning system than that described in the Brun et al. paper as it avoids using the shuttle plasmid pMRB-MG1/pNF. The huSTEAP transgene sequence was ligated into the modified Maraba MG1 genome backbone at its MluI and BsiWI site (at cloning region between G and L). The Maraba-MG1-huSTEAP was then rescued (as previously described in Brun J. et al., (2010) Mol Ther 18: 1440-1449), plaque purified once, and subjected to opti-prep purification. The Maraba-MG1-huSTEAP has a genomic sequence that is the reverse complement and RNA version of SEQ ID NO: 15.

An exemplary priming virus according to the present disclosure is adenovirus type 5 with E1/E3 deletion expressing huSTEAP or murine STEAP (muSTEAP). In tumour-free mice, huSTEAP immunization in tumour free animals using Ad-huSTEAP was successful in generating anti-STEAP immune responses. While the responses had a stronger reactivity to the human peptides, there was evidence of boosted immune responses directed towards epitopes present in the murine STEAP protein. Treatment with Ad:MG1-huSTEAP was able to generate anti-STEAP immune responses and significantly impaired tumour growth leading to significantly improved survival.

The adenovirus, the Maraba MG1 virus, or both, may be formulated for administration as isolated viruses. The adenovirus may be formulated, for example, in 10 mM Tris-Cl, pH 8.0, with 10% glycerol. The Maraba MG1 virus may be formulated, for example, in 10 mM HEPES, 0.15 M NaCl and 4% sucrose at an approximate pH of 7.5.

In combination prime:boost therapies according to the present disclosure, the two viruses may be capable of expressing antigenic proteins, such as HPV E6/E7 fusion proteins or huSTEAP proteins, that do not have identical sequences. For example, the adenovirus may be capable of expressing an HPV E6/E7 fusion protein that has the four protein domains in the order ABCD, while the Maraba MG1 virus may be capable of expressing an HPV E6/E7 fusion protein that has the four protein domains in the order BADC. In another example, the adenovirus may be capable of expressing an HPV E6/E7 fusion protein where the four protein domains are linked by proteasomally degradable linkers that are different from the proteasomally degradable linkers linking the four protein domains of the fusion protein expressed by the Maraba MG1 virus. In still another example, the adenovirus may be capable of expressing a huSTEAP protein according to SEQ ID NO: 13, while the Maraba MG1 virus may be capable of expressing a huSTEAP protein that is variant of SEQ ID NO: 13, such as a protein that is 90% identical to SEQ ID NO: 13.

The term “variant” should be understood to refer to a protein that is at least 70% identical to the sequence of the reference protein. Preferably, the variant will be at least 80% identical. More preferably, the variant will be at least 90% identical. Even more preferably, the variant will be at least 95% identical. In the context of a fusion protein of a specific sequence, such as SEQ ID NO: 1, a variant of the fusion protein would be understood to refer to a protein where each of the protein domains are at least 70% identical to the sequences of their corresponding domains in the reference protein. Preferably, the variant will be at least 80% identical. More preferably, the variant will be at least 90% identical. Even more preferably, the variant will be at least 95% identical. Variants with higher sequence identities have increased likelihood that the epitopes are presented in a similar 3-dimensional manner to the reference protein. Accordingly, in yet another example of combinations where the two viruses do not generate proteins with identical sequences, the adenovirus may be capable of expressing a protein according to SEQ ID NO: 1, while the Maraba MG1 virus may be capable of expressing a protein that is a variant of SEQ ID NO: 1, such as a fusion protein where each of the four protein domains are at least 90% identical to the sequences of their corresponding protein domains in SEQ ID NO: 1.

In the context of the present disclosure, it should be understood that all discussions of, and references to, a ‘protein expressed by a virus’ more exactly refer to a protein expressed by a cell infected with the virus since viruses do not themselves have the capability to express proteins. Similarly, all discussions of, and references to, a ‘virus that expresses a protein’ or ‘virus capable of expressing a protein’ more exactly refer to a virus that includes the genetic information necessary for the protein to be expressed by a cell infected with the virus (i.e. “encodes” the protein).

In the context of the present disclosure, a “combination prime:boost therapy” should be understood to refer to therapies where the adenovirus and the Maraba MG1 virus discussed herein are to be administered as a prime:boost treatment. The adenovirus and the Maraba MG1 virus need not be physically provided or packaged together since the adenovirus is to be administered first and the Maraba MG1 virus is to be administered only after an immune response has been generated in the mammal. In some examples, the combination is provided to a medical institute, such as a hospital or doctors office, in the form of a plurality of packages of the priming adenovirus, and a separate plurality of packages of the boosting Maraba MG1 virus. The packages of adenovirus and the packages of Maraba MG1 virus may be provided at different times. In other examples, the combination is provided to a medical institute, such as a hospital or doctors office, in the form of a package that includes both the priming adenovirus and the boosting Maraba MG1 virus.

The combination prime:boost therapy may additionally include an immune-potentiating compound, such as cyclophosphamide (CPA), that increases the prime immune response to the tumor associated antigenic protein generated in the mammal by administrating the first virus. Cyclophosphamide is a chemotherapeutic agent that may lead to enhanced immune responses against the tumor associated antigenic protein. In a synergistic murine melanoma tumor model, CPA administered prior to the priming vector significantly increased survival, while CPA administered prior to the boosting vector did not.

The therapeutic approach disclosed herein combines: (1) an adenoviral vaccine, and (2) a Maraba MG1 virus as an oncolytic viral vaccine, both expressing an antigenic protein, such as Human Papilloma Virus E6/E7 fusion protein or huSTEAP. Boosting with a oncolytic vaccine of the present disclosure may lead to both tumour debulking by the oncolytic virus and a large increase in the number of tumour-specific CTL (cytotoxic T-lymphocytes) in animals primed by the adenoviral vaccine. Paradoxically, this methodology actually generates larger anti-tumour responses in tumour-bearing animals, as compared to tumour-free animals, since the replication of oncolytic virus is amplified in the tumor-bearing animals, which leads to an increase in the number of antigen-specific Tumour Infiltrating Lymphocytes (TILs), when compared to the replication of oncolytic virus in the tumor-free animals and the associated number of antigen-specific Tumour Infiltrating Lymphocytes (TILs).

The expression products of the HPV gene are processed into peptides, which, in turn, are expressed on cell surfaces. This can lead to lysis of the tumour cells by specific CTLs. The T cell response to foreign antigens includes both cytolytic T lymphocytes and helper T lymphocytes. CD8⁺ cytotoxic or cytolytic T cells (CTLs) are T cells which, when activated, lyse cells that present the appropriate antigen presented by HLA class I molecules. CD4⁺ T helper cells are T cells which secrete cytokines to stimulate macrophages and antigen-producing B cells which present the appropriate antigen by HLA class II molecules on their surface.

The term “mammal” refers to humans as well as non-human mammals. The term “cancer” is used herein to encompass any cancer that expresses, as antigenic proteins, the proteins encoded by the prime and boost viruses, such as E6 and E7 proteins or huSTEAP protein. Examples of such a cancer include, but are not limited to: multiple epithelial malignancies such as cervical cancer, head and neck cancer, and other ano-genital cancers; prostate cancer, pancreatic cancer, colon cancer, breast cancer, testicular cancer, cervical cancer, bladder cancer, ovarian cancer, acute lyphocytic leukemia, and Ewing sarcoma.

The adenovirus, the Maraba MG1 virus, or both may be independently administered to the mammal intravenously, intramuscularly, intraperitoneally, or intranasally. Following administration of the viruses, an immune response is generated by the mammal within an immune response interval, e.g. within about 4 days, and extending for months, years, or potentially life.

To establish an immune response to the antigenic protein, vaccination using the adenovirus and the Maraba MG1 virus may be conducted using well-established techniques. As one of skill in the art will appreciate, the amount of virus required to generate an immune response will vary with a number of factors, including, for example, the mammal to be treated, e.g. species, age, size, etc. In this regard, for example, intramuscular administration of at least about 10⁷ PFU of Adenoviral vector encoding HPV E6/E7 protein to a mouse is sufficient to generate an immune response. A corresponding amount would be sufficient for administration to a human to generate an immune response.

Once an immune response has been generated in the mammal by administration of the adenovirus encoding the antigenic protein, Maraba MG1 virus encoding the antigenic protein is administered in an amount suitable for oncolytic viral therapy within a suitable immune response interval. A suitable immune response interval may be, for example, at least about 24 hours, preferably at least about 2-4 days or longer, e.g. at least about 1 week, or at least about 2 weeks. The amount of Maraba MG1 virus suitable for oncolytic viral therapy will vary with the mammal to be treated, as will be appreciated by one of skill in the art. For example, 10⁸ PFU of Maraba MG1 virus encoding HPV E6/E7 protein administered IV to a mouse is sufficient for oncolytic therapy. A corresponding amount would be sufficient for use in a human.

Maraba MG1 virus encoding HPV E6/E7 protein may be prepared by incorporating a reverse complement of a transgene encoding the HPV E6/E7 protein into the Maraba MG1 virus using standard recombinant technology. For example, the reverse complement of the transgene may be incorporated into the genome of the Marama MG1 virus, or alternatively, may be incorporated into the virus using a plasmid incorporating the transgene. The transgene encoding the protein may be a codon optimized transgene.

An exemplary combination prime:boost therapy according to the present disclosure is shown in the examples to be capable of curing, in mice, the majority of advanced and bulky subcutaneous tumours with a mean volume of 250 mm³. The exemply combination prime:boost therapy is shown to induce tumour specific CD8+ T cell responses in mice with the potential to produce over fifty million E7-specific T cells in the mouse. Without wishing to be bound by theory, the authors of the present disclosure believe that a combination prime:boost therapy according to the present disclosure using HPV E6/E7 as the antigenic protein may be used in humans to treat an HPV-positive tumour.

While the TC1 tumour model used herein does not appear to be susceptible to direct oncolysis, the authors of the present disclosure believe that HPV positive human tumours will be selectively infected and killed by MG1 Maraba. In the absence of type I IFNs, Maraba is able lyse TC1 cells in vitro. However, the pre-treatment of TC1 cells with IFNβ protects these cells from viral oncolysis. The resultant protection by type I IFN explains the lack of efficacy when mice bearing established TC1 tumours are treated with MG1 Maraba encoding a non-specific transgene. Both E6 and E7 inhibit cellular responses to type I IFNs16 when integrated into the genomes of human cells (as is the case in high-grade HPV malignancies). The data from the G-deleted VSV assay (assay discussed below, data not shown) recapitulates this effect in a human epithelial cell line. By confirming the expression of E6 and E7 in the TC1 cell line the lack of susceptibility to viral oncolysis could be explained by the inability of E6 and E7 to interact with the type I IFN cascade in murine tissue, thus such an effect is specific to the adaptation of HPV to its host human organism. Without wishing to be bound by theory, the authors of the present disclosure believe that susceptibility of HPV positive human tumours may further bolster the potency of a combination prime:boost therapy according to the present disclosure.

As Maraba virus is a member of the rhabdovirus family it does not pose a risk of insertional mutagenesis due to the fact DNA is never manufactured in the virus's life cycle, which occurs entirely outside of the nucleus. The frequency of adenoviral integration into the host genome is low. If cells transduced with Ad-E6E7 were to undergo an integration event with an E6E7 transgene that encoded a protein with the optional mutations discussed above that prevent zinc finger formation, the protein produced would be unable to interfere with the functions of p53 or retinoblastoma thereby reducing the possibility of a de novo neoplasm from forming.

By including the full-length sequences of E6 and E7 from HPV 16 and HPV 18, a patient with an HPV associated cancer may be eligible for treatment with a combination prime:boost therapy according to the present disclosure. The combination therapy may be able to elicit responses against multiple potential epitopes.

The efficacy of a vaccine-based therapy in treating an infectious disease is believed to be related to the ability of protective T cells being able to produce multiple cytokines. An exemplary combination prime:boost therapy is shown herein to induce multiple different populations of T cells, defined by their pattern of cytokine production. In some examples, cytokine positive T cells are able to degranulate in the presence of an E7 peptide. The present disclosure illustrates that administration of Ad-E6E7 alone is able to induce multifunctional T cells, but that the numbers of these cells are increased when boosted with MG1-E6E7. Combination prime:boost therapies according to the present disclosure may be used to generate multi-functional T cells, which is believed to be beneficial for a therapeutic vaccine.

As illustrated by the experiments discussed herein, an exemplary combination prime:boost therapy according to the present disclosure may generate specific anti-tumour cytotoxic T cells, and depletion of such CD8+ T cells results in a loss in efficacy.

Without wishing to be bound by theory, the authors of the present disclosure believe that a combination therapy that is able to generate a sizeable specific immune response will result in a favourable clinical outcome, perhaps in the face of advanced disease. The authors of the present disclosure also believe that such a combination therapy may be useful for inducing specific E6 and/or E7 responses in patients that lacked such responses, and that treatment with such a combination therapy may improve a patient's prognosis. In mice cured of advanced TC1 tumours, the exemplary combination therapy tested herein shows marked and durable persistence of reactive CD8+ T cells with a relative expansion of central memory T cells with time. The lack of central memory T cells is considered one potential cause for failure of therapeutic cancer vaccinations. The results discussed herein shows that an exemplary combination therapy according to the present disclosure is capable of treating, in mice, a model of an advanced HPV positive tumour.

### Materials and Methods

Mice

Six to eight week old female C57BL/6 mice were purchased from Charles River (Wilmington, Mass.) and housed in specific pathogen-free conditions. All animal studies were approved by McMaster University's Animal Research Ethics Board and complied with guidelines from the Canadian Council on Animal Care.

Recombinant Viruses

Codon optimised transgenes were specifically manufactured encoding the mutant attenuated E6E7 and WT E6E7 (GensScript, Piscataway, N.J.) sequences. Ad BHG and Ad E6E7 are human serotype 5 replicate deficient (E1/E3 deleted) adenoviruses. Ad BHG contains no transgene, AdE6E7 contains the transgene encoding the attenuated therapeutic E6E7 construct. The GFP or E6E7 transgenes were inserted between the G and L viral genes of the attenuated MG1 strain of Maraba virus to produce MG1 GFP and MG1 E6E7 respectively.

Cell Culture

Murine TC1 cells expressing E6 and E7 from HPV 16 were grown in RPMI containing 10% foetal bovine serum, 10 mmol/l HEPES, 2 mmol/l L-glutamine and 400 μg/ml G418 (Gold Biotechnology, St Lois, Mo.). Vero, L929 and A549 cells were all cultured in αMEM containing 8% foetal bovine serum and 2 mmol/l L-glutamine. SaOS2 cells were cultured in DMEM containing 10% foetal bovine serum and 2 mmol/l L-glutamine. Panc02 cells were cultured in RPMI containing 10% foetal bovine serum and 2 mmol/l L-glutamine.

In Vitro Infections

Six well plates containing confluent TC1 cells (approximately 1.5×10⁶ per well) were infected at decreasing multiplicity of infection (from 10 to 0.001 as well as an uninfected control well) in 200 μl of culture medium for 45 minutes, following infection fresh medium was added and at 48 hours post-infection, cells were fixed with methanol and stained with 0.1% crystal violet (Sigma-Aldridge St Lois, Mo.) in 20% ethanol for viability.

Interferon β Response Test

The IFNβ responsive L929 and IFNβ resistant Panc02 cell lines were plated alongside TC1 cells in a 96 well plate and upon reaching confluence were treated with a dilution series of murine IFNβ overnight. The following day the cells were infected with 5×10⁵ PFU per well of wild type VSV expressing GFP. Fluorescence was detected 24 hours after infection using a Typhoon Trio Variable Mode Imager (GE Healthcare, Buckinghamshire, U.K.).

G Deleted VSV Assay to Determine the Effect of E6 and E7 on Innate Antiviral Response in Human Epithelial Tumour Cells

A549 human lung adenocarcinoma cells were seeded in a 96 well plate and co-transfected, using Lipofectamine 2000 (ThermoFisher Scientific, Waltham, Mass.), with a plasmid of interest in combination with a plasmid encoding the VSV glycoprotein (PSG5-G). Cells were subsequently infected with a G deleted VSV expressing GFP and supernatants were harvested. Supernatants containing any rescued viral progeny were collected and serial dilutions were used to infect confluent Vero cells in a 96 well plate and this was imaged for fluorescence. Only cells that were successfully transfected with PSG5-G and have inhibition of the anti-viral state by the transfected plasmid of interest are able to produce viral progeny as detected by fluorescence.

Transient Transfections

A549 cells plated were in 6 well plates and when 80% confluent, transfected with 2 μgs of wild type E6E7 from HPV 16 and 18, the attenuated E6E7 transgene or GFP in the pShuttle-CMV vector (Agilent, Santa Clara, Calif.) using Lipofectamine 2000 (ThermoFisher Scientific, Waltham, Mass.). SaOS2 cells were cotransfected with HA tagged retinoblastoma in pcDNA 3 (Gift from Joe Mymryk), GFP and one of WT E6E7, attenuated E6E7 or empty pShuttle-CMV. Cells were lysed in 100 μl of radioimmunoprecipitation assay buffer supplemented with Complete Mini protease inhibitor tablets (Roche, Mannheim, Germany) 24-48 hours after transfection.

Western Blotting and Antibodies

Equivalent amounts (20 or 30 μgs) of protein lysate were loaded per lane onto polyacrylamide gels and separated by SDS-PAGE, transferred to 0.45 μm nitrocellulose membrane. Membranes were blocked with either 5% fat-free milk in PBS or Odyssey Blocking Buffer (LI COR Biosciences, Lincoln, Nebr.) for 40 minutes at room temperature. Membranes were probed with antibodies raised against p53 (clone DO1, Santa Cruz, Dallas, Tex.), HA (clone F7, Santa Cruz, Dallas, Tex.), E7 (clone 8E2, Abcam, Cambridge, U.K.), β-actin (clone 13E5, Cell Signalling, Danvers, Mass.) and GFP (clone D5.1, Cell Signalling, Danvers, Mass.). Membranes were then probed with secondary IRDye (LI COR Biosciences, Lincoln, Nebr.) antibodies. Membranes were scanned and had fluorescence quantified using the LI COR Odyssey system (LI COR Biosciences, Lincoln, Nebr.).

Vaccination of Mice

Adenovirus was administered under gaseous general anaesthesia at a dose of 2×10⁸ PFU in 100 μl of 0.9% NaCl for injection (Hospira, Lake Forest, Ill.), the dose was split in two and 50 μl was injected in the semimembranosus muscle of both hind limbs. For direct oncolysis of TC1 tumours Maraba MG1 GFP was injected intravenously at a dose of 5×10⁸ PFU in 200 μl 0.9% NaCl in 3 doses given 48 hours apart. When used as a boost Maraba MG1 GFP or E6E7 was administered at a dose of 1×10⁹ PFU in 200 μl 0.9% NaCl as a single dose 9 days after adenoviral vaccination.

Tumour Challenge

Mice were engrafted with 1×10⁸ TC1 cells subcutaneously under gaseous general anaesthesia. The longest axis of the tumour (length) and the axis perpendicular (width) to this were measured every 2-3 days and tumour volume was calculated using the following formula:

Volume=4/3π(0.5 length×0.5 width²)

Treatment of tumour bearing mice was initiated when a mean tumour volume of 250 mm³ was reached in engrafted mice. Mice reached end point when tumours grew to a volume of 1500 mm³ or the mouse lost 20% of its body weight relative to weight recorded prior to tumour engraftment.

Peptides

Known immunodominant peptides from HPV serotype 16 were synthesised by Biomer Technologies (San Francisco, Calif.). The sequence of H-2K^{b} binding E6 peptide used was EVYDFAFRDL (SEQ ID NO: 16) and the sequence of the H-2D^{b} binding E7 peptide used was RAHYNIVTF (SEQ ID NO: 17).

Intracellular Cytokine Staining and Antibodies

Blood samples were acquired 8 days after adenoviral vaccination and 5 days after Maraba MG1 treatment, spleens were also harvested 5 days post Maraba. Peripheral blood mononuclear cells and splenocytes were incubated in complete RPMI (containing 10% foetal bovine serum and 2 mmol/l L-glutamine) with 2 μg/ml peptide and anti CD107a (clone 1D4B, BD, Franklin Lakes, N.J.). Incubations were performed for a total of 5 hours in a 37° C., 5% CO₂ incubator at 95% humidity, 1 μg/ml of brefeldin A (GolgiPlug, BD, Franklin Lakes, N.J.) was added for the last 4 hours. Cells were then incubated with anti CD16/CD32 (clone 2.4G2, Mouse BD Fc Block, BD, Franklin Lakes, N.J.). T cell surface staining was performed with antibodies against CD8a (clone 53-6.7, eBiosciences, Inc., San Diego, Calif.) and CD4 (clone RM4-5, eBiosciences, Inc., San Diego, Calif.). Cells were subsequently fixed and permeabilised (Cytofix/Cytoperm, BD, Franklin Lakes, N.J.). Intracellular cytokine staining was then performed using antibodies against IFNγ (clone XMG1.2, BD, Franklin Lakes, N.J.), TNFα (clone MP6-XT22, BD, Franklin Lakes, N.J.) and IL-2 (clone JES6-5H4, BD, Franklin Lakes, N.J.). Data were acquired using an LSRFORTESSA cytometer (BD, Franklin Lakes, N.J.) and analysed with FlowJo Mac software (Treestar, Ashland, Oreg.).

T Cell Counts

A known quantity of fluorescent beads (123count eBeads, eBiosciences, Inc., San Diego, Calif.) were added to 50 μl of whole blood which had been stained with antibodies against CD8a (clone 53-6.7, eBiosciences, Inc., San Diego, Calif.) and CD4 (clone RM4-5, eBiosciences, Inc., San Diego, Calif.) and fixed as well as lysed (1-step Fix/Lyse solution, eBiosciences, Inc., San Diego, Calif.). The cells and beads were re-suspended in FACS after 2 wash steps and absolute cell numbers were calculated. For enumeration of splenocytes, the entire spleen was processed and re-suspended in complete RPMI, 50 μl of the re-suspended splenocytes were then analysed as for peripheral blood. Total blood volume in μl was calculated by multiplying each mouse's body weight in grams by 70 thus allowing a total circulating count of T cells.

T Cell Memory Phenotype and Antibodies

PBMCs and splenocytes were incubated with anti CD16/CD32 (clone 2.4G2, Mouse BD Fc Block, BD, Franklin Lakes, N.J.). Cells were then stained with antibodies against CD8a (clone 53-6.7, eBiosciences, Inc., San Diego, Calif.), CD4 (clone RM4-5, eBiosciences, Inc., San Diego, Calif.), CD62L (clone MEL-14, BD, Franklin Lakes, N.J.), CD127 (clone SB/199, BD, Franklin Lakes, N.J.) and the HPV H-2D^{b} E7 tetramer RAHYNIVTF (Baylor College of Medicine, Houston, Tex.).

Depletion Antibodies

T cells were selectively depleted with 2 doses of anti-CD8a (2.43 clone) or anti-CD4 (GK1.5) clone given 48 hours apart. Mice were injected intra-peritoneally with 200 μgs antibody in 300 μls 0.9% NaCl. Depletions were assessed flow cytometrically from peripheral blood samples stained for CD8a and CD4.

Statistical Analysis

Data were graphically displayed and analysed using GraphPad Prism version 6 for Mac (GraphPad Software, San Diego, Calif.). Transfection and immune response data were plotted as column charts with mean and standard error of the mean displayed. Unpaired T-tests were used when comparing two groups and ANOVA tests were used to compare greater than two groups. Survivals were plotted using Kaplan-Meier curves and median survivals were compared using the log-rank tests. Statistical significance was defined as p≤0.05 (*p≤0.05, **p≤0.01, ***p≤0.001, ****p≤0.0001).

### EXAMPLES

For clarity, the HPV E6E7 fusion protein encoded by the viruses in the following examples has a sequence according to SEQ ID NO: 1. It may be referred to as the “attenuated therapeutic E6E7 construct”.

### Example 1

An Attenuated E6E7 Transgene Does Not Degrade p53 or Retinoblastoma In Vitro

One example of an HPV E6/E7 fusion protein according to the present disclosure was designed and cloned into the adenoviral and Maraba MG1 viruses. The exemplary fusion protein was based on the E6 and E7 transforming proteins of the 16 and 18 serotypes of HPV. GGGGGAAY linkers were included between each of the four protein domains to promote proteasomal degradation and generate the HPV16 E6, HPV18 E6, HPV16 E7 and HPV18 E7 proteins. In both of the HPV16 and HPV18 E6 domains, deletion mutations were made to two of the four CXXC motifs which function to form zinc fingers mediating the degradation of p53. In both of the HPV16 E7 and HPV18 E7 domains of the mutated transgene a deletion was applied to one of the carboxy terminus CXXC motifs as well as deletions applied to the LXCXE sequences responsible for the dysfunction of retinoblastoma in HPV induced cancers. The exemplary mutated transgene has a sequence according to SEQ ID NO: 1, and is illustrated in FIG. 1 where the first and second protein domains correspond to HPV16 E6 and HPV18 E6, respectively, the third and fourth protein domains correspond to HPV16 E7 and HPV18 E7, respectively, and the GGGGGAAY linkers are not highlighted.

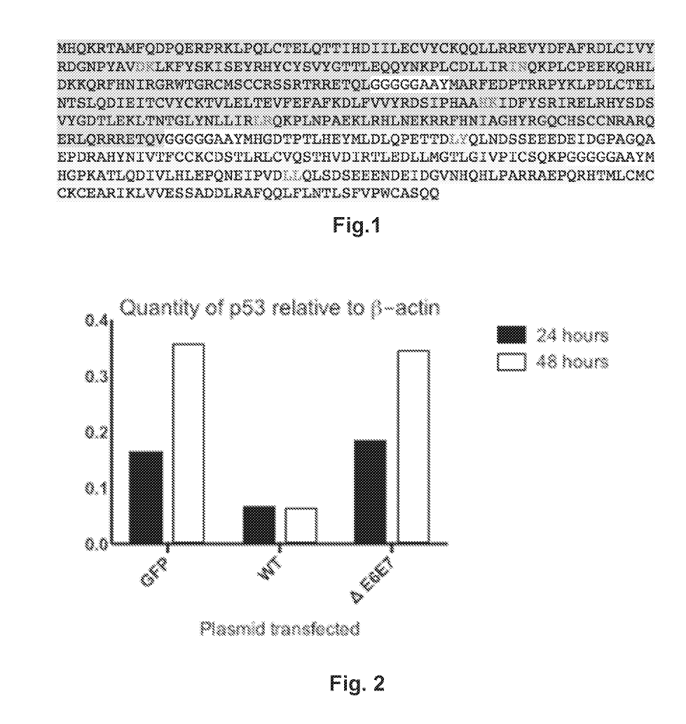

A549 cells containing wild type p53 were transfected with expression vectors containing wild type sequences of the E6E7 transgene, the mutated E6E7 transgene, or an irrelevant control plasmid (GFP) and subsequently levels of p53 were quantified by western blotting. Degradation of p53 was noted with the wild type transgene sequence. However, this activity was inhibited by the mutations introduced to the therapeutic transgene, discussed above, relative to the control (GFP) plasmid. The western blot data for the experiments associated with p53 levels is summarized in FIG. 2, which shows that the expression of the wildtype E6E7 transgene leads to p53 degradation, whereas the mutant E6E7 leaves p53 protein levels intact.

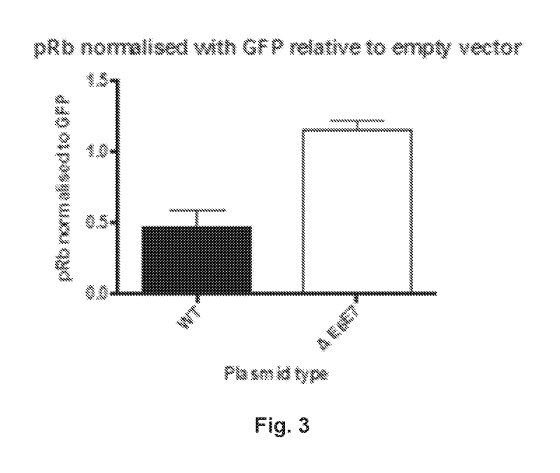

In a parallel set of experiments, the retinoblastoma-null cell line SaOS2 was co-transfected with an expression plasmid encoding HA-tagged retinoblastoma (pRb) alongside a GFP encoding plasmid as well as one of the following three expression vectors encoding: the WT E6E7 transgene, the mutated therapeutic transgene, or a control plasmid. Following transfection, levels of HA-tagged retinoblastoma were quantified using western blot. A significant decrease in the WT E6E7 transfected cell lysates was observed whereas the control plasmid and the mutant E6E7 expression had no effect on steady-state pRb levels. A standardization graph illustrating the western blot data is shown in FIG. 3.

In summary, deletions of specific amino acids in the wild type sequences prevent the transforming activity of the E6 and E7 proteins with relation to their interactions with the p53 and retinoblastoma tumour suppressor proteins respectively, reducing the carcinogenic potential of the vaccine vectors when used in vivo.

### Example 2

### Construction and Immune Testing of Adenovirual Priming Vector and Maraba MG1 Vaccine Vector Expressing a Human Papilloma Virus E6/E7 Fusion Protein

In an exemplary method according to the present disclosure, an oncolytic vaccination strategy was tested. The exemplary method used an adenoviral prime encoding the therapeutic E6E7 (Ad-E6E7) transgene followed by the MG1 Maraba virus boost (MG1-E6E7) encoding the same transgene. The viruses were administered to mice and immune responses were quantified using intracellular cytokine staining (ICS). For comparison sham prime (Ad-BHG) and boost (MG1-GFP) groups were also analysed.

As noted above in the materials and method section, Ad-BHG and Ad-E6E7 are human serotype 5 replicate deficient (E1/E3 deleted) adenoviruses. Ad BHG contains no transgene, Ad-E6E7 contains the transgene encoding the attenuated therapeutic E6E7 construct. The GFP or E6E7 transgenes were inserted between the G and L viral genes of the attenuated MG1 strain of Maraba virus to produce MG1-GFP and MG1-E6E7 respectively.

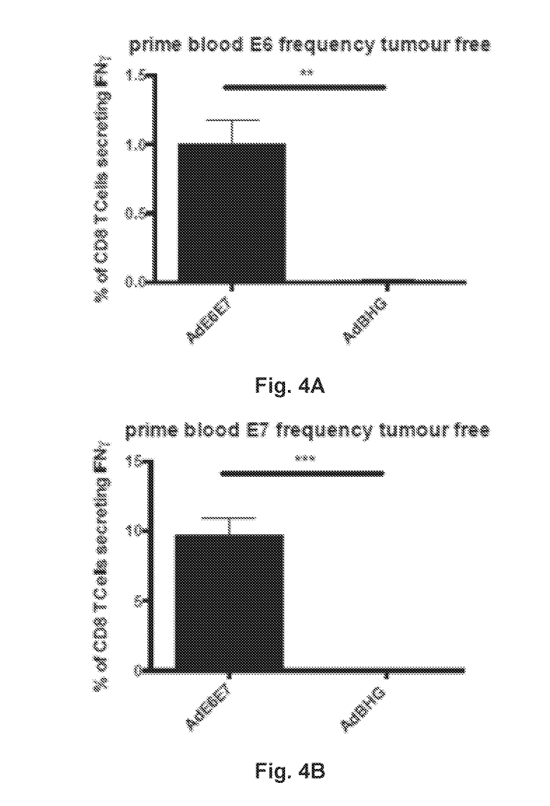

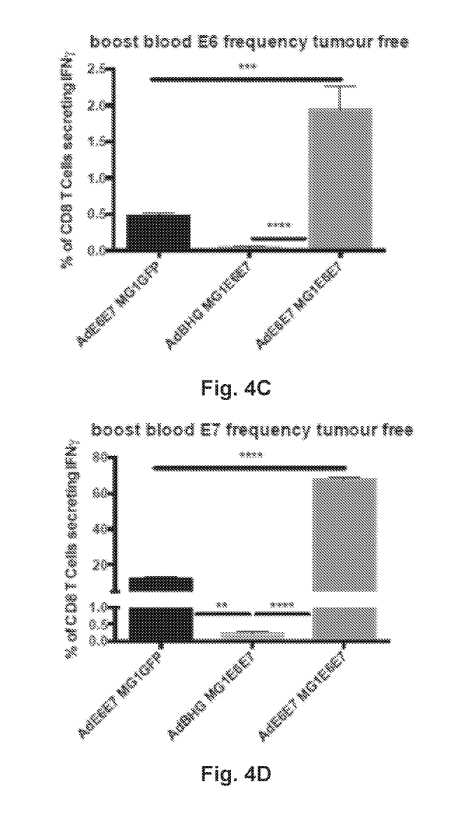

Peripheral blood mononuclear cells were re-stimulated with known E6 (EVYDFAFRDL) and E7 (RAHYNIVTF) C57BL/6 CD8+ epitopes. Blood samples after the Ad-E6E7 prime revealed that specific responses were generated against both the E6 and E7 epitopes by the production of interferon-γ (IFNγ) from CD8+ T cells. About 1% of CD8+ T cells secreted IFNγ on stimulation with the E6 epitope, and about 10% of CD8+ T cells secreted IFNγ on stimulation with the E7 epitope. No responses were seen after the sham prime. Small responses were seen after MG1 E6E7 was administered following the sham prime (mean frequency of 0.042% and 0.21% of CD8+ T cells producing IFNγ for the E6 and E7 peptides respectively). However, mice administered MG1 E6E7 following the Ad-E6E7 prime showed a significant increase in IFNγ+ T cell frequency. Again the E7 epitope appeared to be dominant with a mean frequency of 68.87% of CD8+ T cells producing IFNγ following restimulation with this peptide. These data are summarized in FIGS. 4A-4D.

In order to compare the true magnitude of the immune responses generated with AdE6E7 alone compared to the Ad-E6E7 prime: MG1-E6E7 boost, a subset of mice were sacrificed following the time boosting, ICS was again performed following restimulation with the E7 peptide and the number of peripheral blood and splenic CD8+ T cells were quantified using fluorescent microparticle beads specifically designed for enumerating cell numbers flow cytometrically.

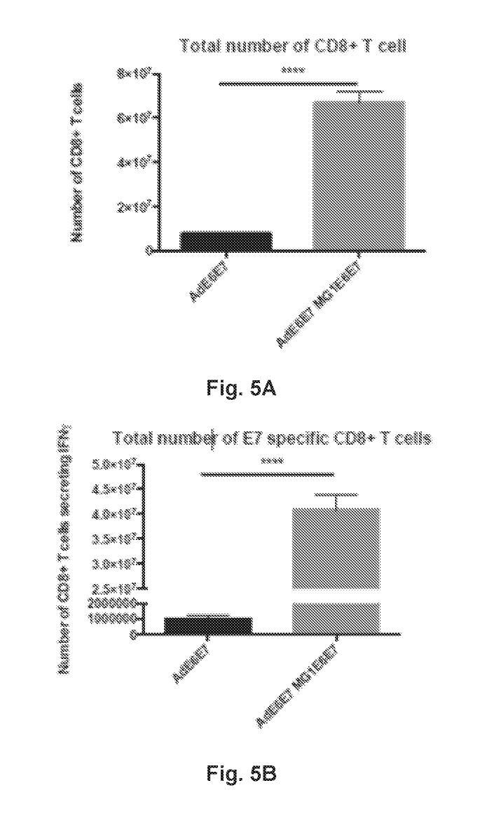

The Ad-E6E7 prime: MG1-E6E7 boost induced a marked and highly significant expansion of total and E7 specific CD8+ T cell populations compared to the AdE6E7 alone, as illustrated in FIG. 5. The Ad-E6E7 prime: MG1-E6E7 boost generated 6.5×10⁷ total number of CD8+ T cells. When the splenic and circulating pools of E7 specific CD8+ T cells were combined a mean absolute count of 4.1×10⁷ was generated following the Ad-E6E7 prime: MG1-E6E7 boost (n=10, range=2.4-5.1×10⁷). Combination prime:boost therapy using an exemplary HPV E6E7 fusion protein as the tumour antigen was able to generate specific CD8+ T cell responses against E6 and E7 epitopes with an extensive expansion of effector cells.

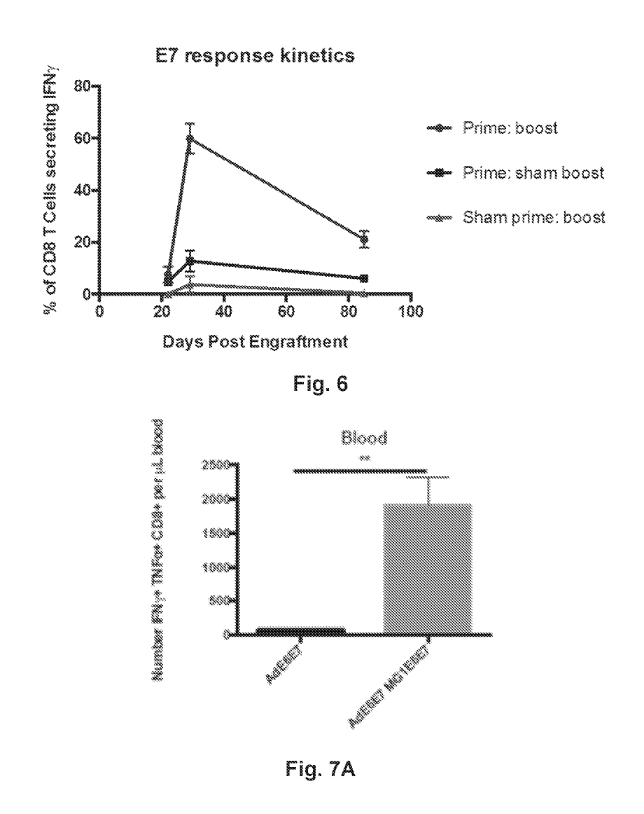

The response kinetics was also measured for the exemplary combination prime:boost therapy. CD8+ T cell responses exceeding 50% of all peripheral CD8+ T cells at peak. At a later time point, greater than 20% of peripheral CD8+ T cells responded to the transgene, as measured against a single known C57/B6 epitope in E7. This data is illustrated in FIG. 6.

### Comparative Example 3

### Construction and Immune Testing of Lentiviral Priming Vectors and Oncolytic Vaccine Vectors Expressing Human Papilloma Virus E6/E7 Fusion Protein

Lentivirus expressing HPV E6/E7 was tested as a comparative priming virus.

The HPV transgene is a fusion of HPV serotype 16 full-length wild-type E6 (gi/4927720/gb/AAD33252.1/AF125673_1 E6 Human papillomavirus type 16) and E7 (gi/4927721/gb/AAD33253.1/AF125673_2 E7 Human papillomavirus type 16) sequences and HPV serotype 18 full-length wild-type E6 (gi/137758/sp/P06463.1/VE6_HPV18 RecName: Full=Protein E6) and E7 (gi/137792/sp/P06788.2/VE7_HPV18 RecName: Full=Protein E7) sequences with deletions in all 4 nucleotide sequences to remove zinc fingers required for Rb or p53 binding (removing oncogenic potential of the proteins). The resulting fusion protein has a flexible glycine linker plus AAY sequence (which serves as a proteasomal cleavage site to ensure that each antigen is proteolytically degraded to the peptides normally generated for antigen presentation). This codon-optimized fusion nucleotide sequence gives rise to a 527 amino acid HPV16/18 E6/E7 fusion protein (SEQ ID NO: 1).

Lentiviruses expressing Human Papilloma Virus E6/E7 fusion transgene were made using the pDY.EG.WS lentivirus vector. The modified HPV transgene was PCR amplified using primers containing the EcoRI restriction site (forward primer ACTGGAATTCATGCATCAGAAGCGAACTGC, SEQ ID NO: 18) and the BamHI restriction site (reverse primer ACTGGGATCCTCACTGCTGGGAGGCACAC, SEQ ID NO: 19). The HPV transgene PCR product was agarose gel purified. The pDY.EG.WS lentivirus vector was cut at the EcoRI and BamHI sites to remove eGFP, was agarose gel purified, and was subjected to dephosphorylation using CIAP (Invitrogen Catalogue 18009-019). The cut vector was then subjected to additional agarose gel purification. The HPV transgene PCR product was then ligated into the EcoRI/BamHI cut vector using T4 DNA ligase (Invitrogen). The ligation reaction was subjected to a transformation using competent cells, and plasmid DNA from positive colonies was subjected to mini-prep amplification. The pDY.EG.WS lentivirus vector expressing the modified HPV transgene was then subjected to maxi-prep amplification. The lentivirus expressing Human Papilloma Virus E6/E7 fusion transgene were rescued on 293T cells after transfection of 6.4 μg of each of three plasmids: the pDY.EG.WS lentivirus vector expressing the modified HPV transgene, the packaging pCMV-8.84 plasmid, and the envelope pMD2G plasmid. Virus supernatants were pooled, and filtered through a 0.45 μM filter and centrifuged for 120 minutes at 50,000×g at 16° C. The lentivirus expressing Human Papilloma Virus E6/E7 fusion transgene was resuspended in PBS, and stored at −80° C.

Maraba MG1 was engineered to contain a Papilloma Virus E6/E7 fusion transgene inserted between the G and L viral genes of the MG1 double mutant of Maraba virus (Brun J. et al., (2010) Mol Ther 18:1440-1449). The transgene sequence (SEQ ID NO: 2) was codon optimized for expression in mammalian cells. The resulting Maraba MG1 containing the HPV E6/E7 is designated, generally, “Maraba-MG1-HPV E6/E7”. A modified Maraba MG1 backbone was used to facilitate cloning. A silent mutation was introduced into the L gene of the Maraba MG1 genome backbone to remove one of the MluI sites. The second MluI site was replaced with a site at the cloning region between G and L. These modifications to the Maraba MG1 genome backbone allowed for a more direct cloning system than that described in the Brun et al. paper as it avoids using the shuttle plasmid pMRB-MG1/pNF. The HPV E6/E7 fused transgene sequence was ligated into the modified Maraba MG1 genome backbone at its MluI site and BsiWI site (at cloning region between G and L) The Maraba-MG1-HPV E6/E7 was then rescued (as previously described in Brun et al., (2010) Mol Ther 18:1440-1449), plaque purified once, and subjected to opti-prep purification.). The Maraba-MG1-HPV E6/E7 used in this example has a genomic sequence that is the reverse complement and RNA version of SEQ ID NO: 4.

Generally, animals were immunized by administration of the priming vector (lentivirus-HPV E6/E7+ poly I:C as an adjuvant) at day 0 and by administration of 1e9 PFU of the boosting vector (Maraba-MG1-HPV E6/E7) at day 14. Control animals were prime:boosted with viral vectors encoding GFP instead of the HPV E6/E7 transgene as a control non-immunogenic transgene insertion. Analysis of the prime response was conducted at day 14 and of the boost response at day 19. Each lentivirus-HPV E6/E7 preparation was made with 250 ug poly I:C added as an adjuvant to the priming virus and then split between 5 animals for each virus. Mice were anesthetized with isoflurane and 30 uL of lentivirus-HPV E6/E7/poly I:C was injected into each hind foot pad. The remaining virus was injected subcutaneously near the left inguinal lymph node. 14 days after prime, blood was collected and analyzed by flow cytometry. Mice were then boosted with 1×10⁹ PFU MG1-HPV E6/E7 intravenously. 5 days following the boost, blood was drawn and immune responses were assessed by flow cytometry.

Immune analysis was performed as follows: Blood was collected via retro-orbital bleeding using heparinzied capillary tube and blood was collected into heparin. Red blood cells were then lysed using ACK lysis buffer and the resulting PBMCs were analyzed for immune responses to the tumour antigens. PBMCs were either incubated in the absence of peptide or stimulated with 2 ug/mL peptides (RAHYNIVTF) for a total of 5 hours with golgi plug added 1 hour into the stimulation. Following stimulation the PBMCs were stained for CD4, CD8 and IFNγ and analyzed on FACSCanto and FlowJo. Responding T-cells were detected after intracellular cytokine staining (ICS) for IFN-γ by flow cytometry. Values from unstimulated PBMCs were considered background and subtracted from values obtained from stimulated PBMCs. Data represents mean +/−SEM. In Table 2 it is demonstrated that the HPV E6/E7 peptides were able to stimulate IFN-γ production in CD8 cells indicating the existence of an immune response.

TABLE 2
IMMUNE RESPONSE TO A COMPARATIVE
COMBINATION THERAPY (LENTIVIRUS HPV-E6E7 AS PRIME)

| | Percentage of CD8 T Cells Secreting Interferon (IFN) γ | | | |
|---|---|---|---|---|
| | | | Immune Group | |
| | | | Lentivirus-HPV | |
| Stimulatory | Control Group | | E6/E7 Prime | |
| Peptide | Lentivirus-GFP Prime | | MG1-HPV E6/E7 Boost | |
| Epitope | MG1-GFP Boost | | (N = 5) | |
| RAHYNIVTF | 0.0033 ± | 0.03 ± 0.025 | 0.036 ± 0.012 | 5.9 ± 2.7 |
| | 0.0033 | (after boost) | (after prime) | (after |
| | (after prime) | | | boost) |

### Example 4

### Oncolytic Vaccination Generates Multifunctional T Cells

To further assess the quality of the generated T cell responses, multifunctional T cell analysis was performed on blood and splenic tissue of mice vaccinated with the Ad-E6E7 prime: MG1-E6E7 boost following restimulation with the E7 peptide. A group of mice receiving AdE6E7 alone was used for comparison.

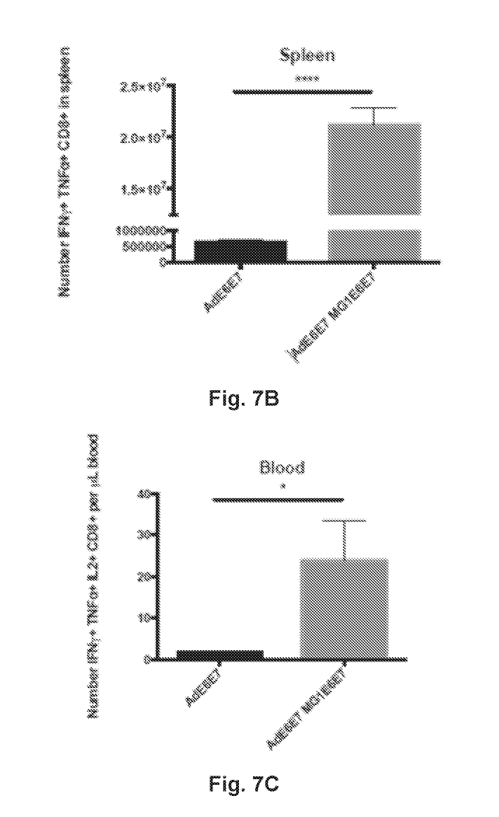

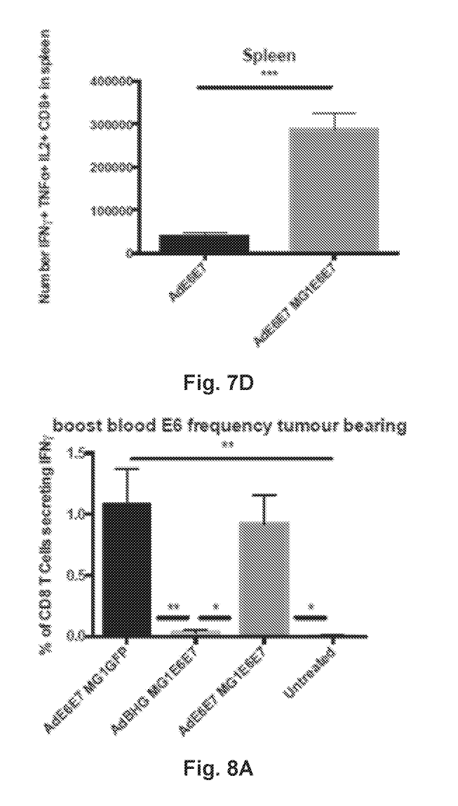

Vaccination with AdE6E7 either alone or followed by the MG1-E6E7 was able to generate double positive (IFNγ and TNFα) and triple positive (IFNγ, TNFα and IL-2) CD8+ T cells found in the circulatory and splenic pools. These data are illustrated in FIGS. 7A-7D.

As illustrated by the data in FIGS. 7A-7D, when the circulatory and splenic, double and triple positive CD8+ T cells were enumerated, mice receiving the Ad-E6E7 prime: MG1-E6E7 boost regimen had significantly more of the two populations in both locations. The degranulation marker, CD107a (LAMP1), was also included in the multifunction analysis and virtually all cells that produced any cytokine in response to the E7 peptide were positive for this marker. Vaccination with AdE6E7 generated CD8+ T cells capable of secreting multiple effector cytokines in response to an E7 peptide, albeit in small numbers, when such mice received the MG1 E6E7 boost a dramatic and significant expansion was seen in these populations.

### Example 5

### Oncolytic E6E7 Vaccination Cures Mice Bearing an Advanced Model of HPV Positive Carcinoma in a CD8+ Dependent Manner

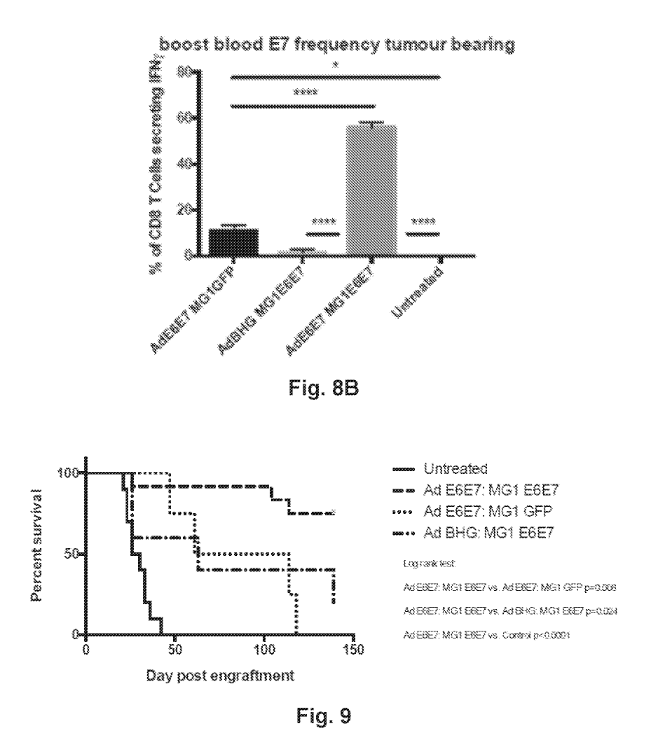

The C57BL/6 cell line TC1 was acquired as a murine model of HPV induced cancer and expression of the E6 and E7 antigens was confirmed by RT-PCR. Following subcutaneous engraftment, mice received the Ad-E6E7 prime when their tumours reached an advanced volume (250 mm³). Intracellular staining in tumour bearing animals revealed specific CD8+ T cell responses against both the E6 and E7 peptides. Moreover a significant expansion of E7 specific T cells was documented after boosting with MG1-E6E7 relative to all other groups. These data are shown in FIGS. 8A and 8B.

Tumour free mice that received the same treatment therapy and control therapies exhibited similar results (data not shown).

Spontaneous immunity was not noted in untreated animals. Mice were sacrificed when either end-point volume was reached (1500 mm³) or they lost 20% of their body mass due to cachexia.

All untreated mice succumbed to tumour progression whereas treatment with either: sham Ad-BHG prime: MG1-E6E7 boost; or Ad-E6-E7 prime: sham MG1-GFP boost, delayed tumour progression but were infrequently curative. However, treatment of mice bearing advanced TC1 tumours with Ad-E6E7 prime followed by MG1-E6E7 boost resulted in durable cures in 75% of mice (n=12). The percent survival vs. time is illustrated in FIG. 9.

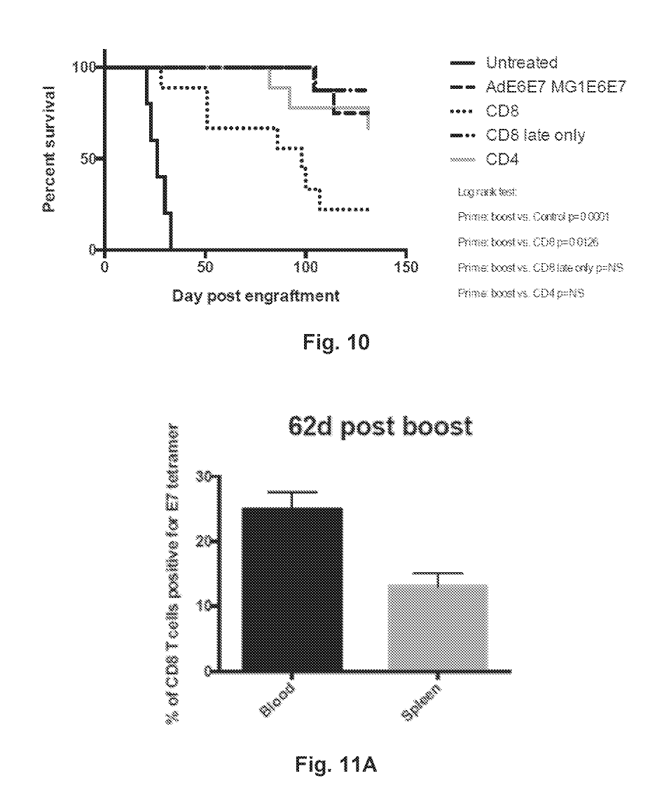

In mice treated with the curative regimen, depletion of CD8+ T cells two days before boosting and subsequently forty days after the boost (“CD8”) resulted in loss of tumour control, no such effect was seen when CD8+ cells were depleted at the later time point alone (“CD8 late”), or when CD4+ cells were depleted two days prior to boosting (“CD4”). Controls included mice that were not treated with a combination prime:boost therapy, and mice that were treated with the exemplary combination prime:boost therapy but did not have CD8 or CD4 cells depleted during the treatment regimen. The percent survival vs. time is illustrated in FIG. 10. Depletion of the CD8+ T cells was accomplished using anti-CD8a (2.43 clone) or anti-CD4 (GK1.5) clone, as discussed above in the methods section.

The exemplary combination prime:boost therapy using Ad-E6E7 as the prime and MG1-E6E7 as the boost is able to generate specific immunity against E6 and E7 antigens in a murine model of HPV with advanced measurable disease leading to durable cures in a CD8+ dependent manner.

### Example 6

### Cured Mice Have Long Lasting Antigen Specific Immunity With a Predominance of Memory CD8+ T Cells

A subset of the mice from Example 5 that were cured from advanced TC1 tumours had further immune analysis performed to assess CD8+ T cell memory phenotype. Circulatory and splenic T cells from cured mice were labelled with an E7 specific tetramer (RAHYNIVTF on H-2D(b)), staining was performed for CD62L and CD127 at 62 days and 117 days after MG1-E6E7 boosting.

Immune analysis revealed a significant persistence of E7 specific CD8+ T cells at both long-term time points in the blood and spleen. The majority of the specific cells were of the effector memory phenotype, with their distribution between blood and spleen illustrated in FIGS. 11A and 11B.

The relative proportions of central memory T cells increased in concert with the interval between boosting and analysis (5.1% in blood and 9.4% in spleen at 62 days post boost compared to 11.8% in blood and 18.8% in spleen at 117 days post boost). The data is shown in Table 5.

TABLE 5
MEMORY PHENOTYPE 62 AND 117 DAYS POST BOOST

| | 62 days | | 117 days | |
|---|---|---|---|---|
| | Blood | Spleen | Blood | Spleen |
| Percentage of E7 tetramer+ cells | 32.7% | 19.5% | 25.5% | 16.8% |
| that are T-effector cells | | | | |
| Percentage of E7 tetramer+ cells | 61.3% | 70.4% | 61.9% | 63.7% |
| that are effector memory cells | | | | |
| Percentage of E7 tetramer+ cells | 5.1% | 9.4% | 11.8% | 18.8% |
| that are central memory cells | | | | |

Oncolytic E6E7 vaccination generates long lasting CD8+ immune memory in mice cured from advanced TC1 tumours.

### Example 7

### Assessment of Induction of Specific Immune Responses in Tumour Free Mice

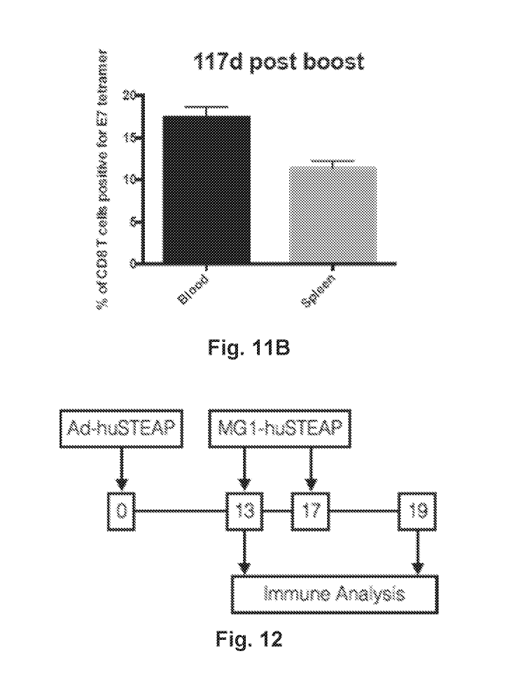

Female C57BI/6 mice were administered Ad-huSTEAP at a total dose of 2e8 PFU i.m. (50 uL of 1e8 PFU given i.m. into each leg). MG1-huSTEAP was administered i.v. at a dose of 1e9 PFU according to the treatment schema illustrated in FIG. 12.

Immune analyses were performed at day 13 (pre-boost) and day 19 (peak boost). Immune analyses were completed on PBMCs by ex vivo peptide re-stimulation and were stained for a panel of cytokines to assess the quantity of STEAP-specific CD8 T cells. All peptides listed in Table 1 were used individually to determine which peptides T cells were responding to and if they were human transgene specific or able to cross-react with mouse sequence.

TABLE 1

| | | Human/mouse | Re-stimulation |
|---|---|---|---|
| Peptide | Sequence | peptide | concentration |
| 186-193 | RSYRYKLL (SEQ ID NO: 20) | Fully conserved | 5 ug/mL |
| hu327-335 | VTKINKTEI (SEQ ID NO: 21) | Human | 5 ug/mL |
| mu327-335 | VSKINRTEM (SEQ ID NO: 22) | Mouse | 5 ug/mL |
| 5-13 | KDITNQEEL (SEQ ID NO: 23 | Fully conserved | 5 ug/mL |

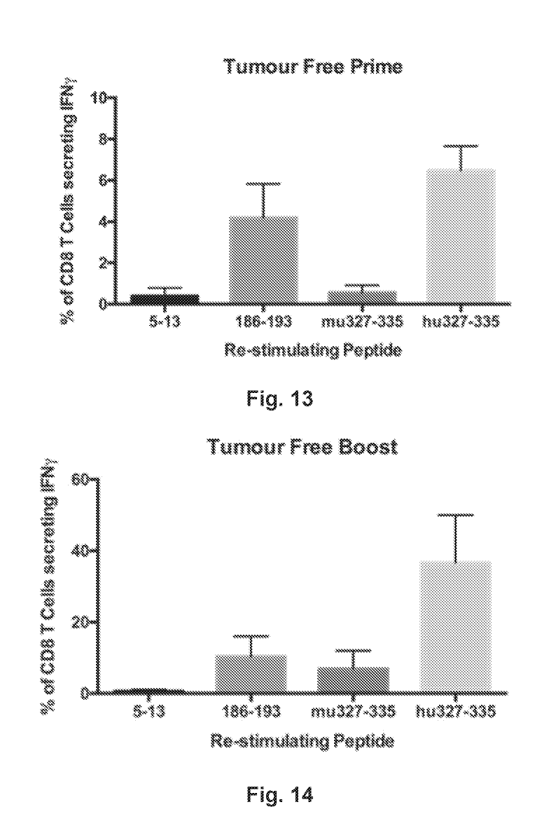

Ad-huSTEAP immunization was able to prime immune responses. The largest response was observed in the hu327-335 peptide re-stimulation. There were also immune responses observed in the mu327 re-stimulation, as well as the conserved 186-193 and 5-13 re-stimulations. These data are illustrated in FIG. 13. Following MG1-huSTEAP, boosted immune responses were observed with all peptides except for the conserved 5-13 peptides. As with the prime immune analysis, the hu327-335 re-stimulation led to the largest anti-STEAP responses. These data are illustrated in FIG. 14.

### Example 8

### Immune Induction and Efficacy in TrampC2 Prostate Cancer Model

Male C57BI/6 mice were engrafted with 2.5e6 TrampC2 cells s.q. on the left flank and tumours were allowed to grow for 33 days. Mice were assigned to one of the three groups shown in Table 2. Since male mice were used in this experiment, mice cannot be swapped in cages. To achieve optimal tumour volume starting point (mean and variance) two cages were combined into one group to achieve the desired mean tumour volume.

TABLE 2

| | | Ad, | MG1 | |
|---|---|---|---|---|
| Group | Group name | Day 0 | Days 8 + 11 | Number |
| 1 | Control | | | 9 |
| 2 | Ad-BHG | ✓ | ✓ | 8 |
| | MG1-GFP | | | |
| 3 | Ad-huSTEAP | ✓ | ✓ | 9 |
| | MG1-huSTEAP | | | |

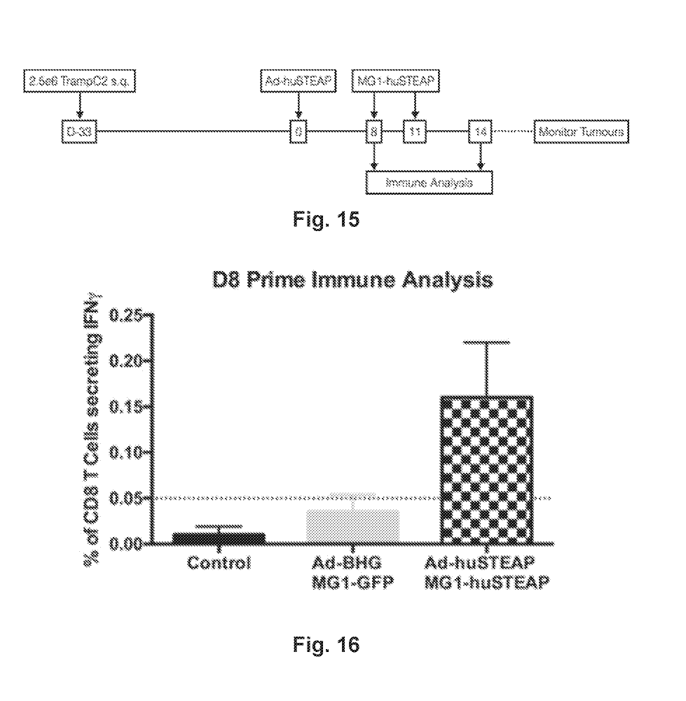

Ad-huSTEAP was administered at a total dose of 2e8 PFU i.m. (50 μL of 1e8 PFU given i.m. into each hind leg). MG1-huSTEAP was administered i.v. at a dose of 1e9. The treatment schema is illustrated in FIG. 15.

Immune analyses were performed on day 8 (prime analysis) and day 14 (peak boost). Immune analyses were completed on PBMCs by ex vivo peptide re-stimulation and were stained for a panel of cytokines to assess the quantity of STEAP-specific CD8+ T cells. Re-stimulation of PBMCs was conducted using a pool of mouse specific peptides (186-193, mu327-335, 5-13, see Table 1).

Survival was recorded for all mice. Mice were considered at endpoint when tumour volumes reached 1500 mm³. To calculate tumour volumes, the following formula was utilized: Volume=(4/3)*3.14159*(L/2)*((W/2)²).

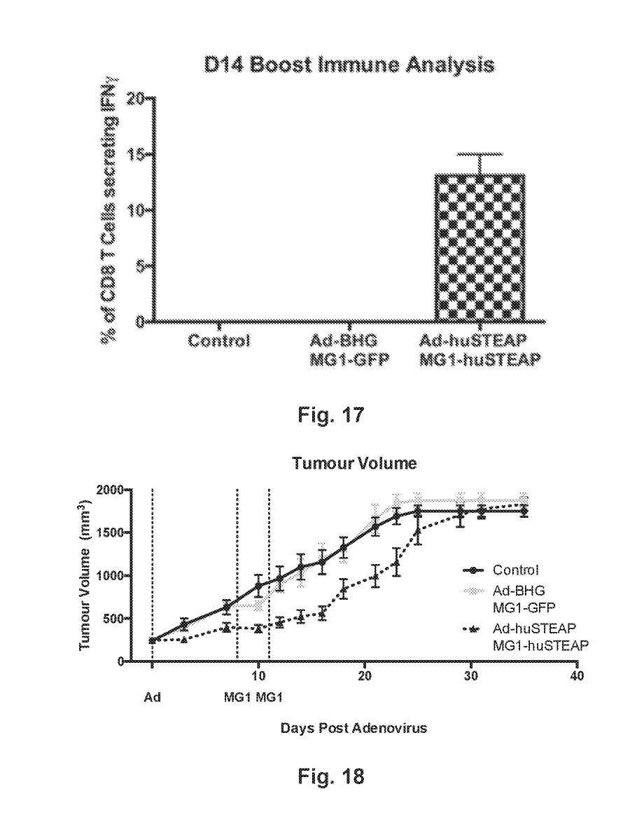

The mean immune response generated following Ad-huSTEAP was diminished compared to that of the tumour free studies (0.15% vs. 5%). However, following MG1-huSTEAP administration, the mean boost response of approximately 15% was closer to that observed in the tumour free experiment (approximately 17%). These data are illustrated in FIGS. 16 and 17, respectively.

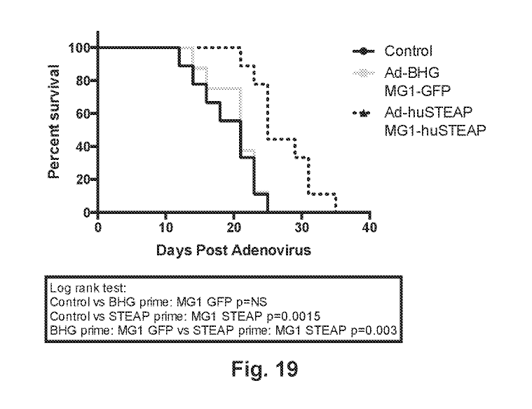

The TrampC2 tumour growth was blunted by the Ad-huSTEAP within three days of Ad-huSTEAP administration, as illustrated in FIG. 18. Tumour control continued for another 10 days following MG1-huSTEAP when the tumours began to slowly grow out. The MG1-GFP however, only had a very limited effect with a small 2-3 day plateau in tumour growth after which the tumours grew back to control sizes and continued to track with the control animals through the rest of the experiment. Treatment with Ad-BHG/MG1-GFP had no impact on survival. However, treatment with with Ad:MG1-huSTEAP led to a significant survival advantage as can be seen in FIG. 19.

In the preceding description, for purposes of explanation, numerous details are set forth in order to provide a thorough understanding of the examples. The above-described examples are intended to be exemplary only. Alterations, modifications and variations can be effected to the particular examples by those of skill in the art without departing from the scope, which is defined solely by the claims appended hereto.

APPEDDIX A
Protein and Nucleotide Sequences

Protein sequence of HPV E6/E7fusion protein (SEQ ID NO: 1):
MHQKRTAMFQDPQERPRKLPQLCTELQTTIHDIILECVYCKQQLLRREVYDFAFRDLCIVYRDGNP
YAV [symbol] LKFYSKISEYRHYCYSVYGTTLEQQYNKPLCDLLIR [symbol] QKPLCPEEKQRHLDKKQRFHNIR
GRWTGRCMSCCRSSRTRRETQLGGGGGAAYMARFEDPTRRPYKLPDLCTELNTSLQDIEITCVYCK
TVLELTEVFEFAFKDLFVVYRDSIPHAA [symbol] IDFYSRIRELRHYSDSVYGDTLEKLTNTGLYNLLIR [symbol]
QKPLNPAEKLRHLNEKRRFHNIAGHYRGQCHSCCNRARQERLQRRRETQVGGGGGAAYMHGDTP
TLHEYMLDLQPETTDLYQLNDSSEEEDEIDGPAGQAEPDRAHYNIVTFCCKCDSTLRLCVQSTHVD
IRTLEDLLMGTLGIVPICSQKPGGGGGAAYMHGPKATLQDIVLHLEPQNEIPVDLLQLSDSEEEND
EIDGVNHQHLPARRAEPQRHTMLCMCCKCEARIKLVVESSADDLRAFQQLFLNTLSFVPWCASQQ*
Note: the diamino acids identified in bold/italics (i,e DK, IN, HK
and LR) correspond to the XX amino acids of CXXC motifs otherwise
found in the wild type sequences, where the cysteines have been
deleted; and the diamino acids identified with underline
correspond to the LX amino acids of LXCXE motifs otherwise found
in the wild type sequences, where the CXE amino acids have been
deleted.
DNA sequence of HPV E6/E7 fusion protein (SEQ ID NO: 3):
ATGCATCAGAAGCGAACTGCTATGTTTCAGGACCCTCAGGAGCGGCCGCGCAAACTGCCTCAGCTG
TGCACCGAACTGCAGACAACTATCCACGACATCATTCTGGAATGCGTGTACTGTAAGCAGCAGCTG
CTGAGGAGAGAGGTCTATGACTTCGCTTTTCGCGATCTGTGCATCGTGTACCGAGACGGAAACCCA
TATGCAGTCGATAAGCTGAAGTTCTACAGCAAGATCTCCGAATACAGGCATTACTGTTACAGCGTG
TACGGGACCACACTGGAGCAGCAGTATAACAAGCCCCTGTGCGACCTGCTGATCAGAATTAATCAG
AAGCCCCTGTGCCCTGAGGAAAAACAGAGGCACCTGGATAAGAAACAGAGATTTCATAACATCCGA
GGACGATGGACCGGGCGGTGCATGTCCTGCTGTAGAAGCTCCCGGACTCGACGAGAGACCCAGCTG
GGCGGAGGAGGAGGAGCAGCTTACATGGCACGATTCGAGGACCCTACCCGAAGGCCATATAAGCTG
CCCGACCTGTGCACAGAACTGAATACTTCTCTGCAGGACATCGAGATTACATGCGTGTACTGTAAA
ACCGTCCTGGAGCTGACAGAAGTGTTCGAGTTTGCTTTCAAGGACCTGTTTGTGGTCTACCGGGAT
TCAATCCCTCACGCAGCCCATAAAATCGACTTCTACAGCAGGATCAGGGAACTGCGCCACTACTCC
GACAGCGTGTACGGGGATACACTGGAGAAGCTGACAAACACTGGCCTGTACAATCTGCTGATCCGA
CTGCGACAGAAGCCACTGAACCCAGCCGAAAAACTGAGACACCTGAACGAGAAGAGACGGTTTCAC
AATATTGCAGGCCATTATAGGGGACAGTGCCATAGTTGCTGTAATCGAGCCAGGCAGGAAAGACTG
CAGCGCCGAAGGGAGACTCAAGTCGGCGGAGGAGGAGGAGCTGCATACATGCACGGCGACACCCCC
ACACTGCATGAATATATGCTGGATCTGCAGCCTGAGACTACCGACCTGTACCAGCTGAACGATTCT
AGTGAGGAAGAGGACGAAATCGACGGACCAGCAGGACAGGCAGAGCCTGACCGGGCCCACTATAAT
ATTGTGACATTCTGCTGTAAGTGCGATTCTACTCTGCGGCTGTGCGTGCAGAGTACTCATGTCGAC
ATCCGCACCCTGGAGGATCTGCTGATGGGGACTCTGGGCATCGTCCCAATTTGTAGCCAGAAACCA
GGCGGCGGCGGCGGAGCAGCTTACATGCACGGACCCAAGGCTACCCTGCAGGACATCGTGCTGCAT
CTGGAACCTCAGAATGAGATTCCAGTCGACCTGCTGCAGCTGAGTGATTCAGAAGAGGAAAACGAC
GAGATCGACGGCGTGAATCACCAGCATCTGCCTGCTAGACGGGCAGAGCCACAGCGACACACAATG
CTGTGCATGTGCTGTAAGTGTGAAGCCAGGATCAAGCTGGTGGTCGAGTCAAGCGCCGACGATCTG
CGCGCCTTCCAGCAGCTGTTCCTGAATACTCTGTCATTTGTCCCTTGGTGTGCCTCCCAGCAGTGA
Exemplary Maraba MG1 DNA sequence that encodes an HPV-E6E7 fusion protein
(SEQ ID NO: 4)

| | |
|---|---|
| acgaagacaa acaaaccatt gataaaatta agaggctcat gaaaatcctt aacagcgttc | 60 |
| aaaatgtctg ttacagtcaa gagagtcatt gatgattcac tcatcacccc caaattgcct | 120 |
| gcqaatgagg accctgtqga gtaccctgct gattatttca aaaagtcccg tgatattccg | 180 |
| gtgtacataa acacgaccaa aagtttgtct gatttgcggg gctatgttta tcaaggccta | 240 |
| aagtcaggca acatctctat aattcatgtc aacagttatc tgtatgcagc attaaaagag | 300 |
| atcagaggaa aattggacag agattggatc acctttggta tccaaatcgg aaaaacagga | 360 |
| gatagcgtgg ggatattcga tttactgacc ctaaaacctc tagatggtgt tttaccagat | 420 |
| ggggtgtctg atgctactcg aactagctca gacgatgcat ggcttccact gtatctattg | 480 |
| gggttataca gagttggtcg aacdcagatg ccagaataca ggaagaagct gatggatggt | 540 |
| ctgattaatc aatgtaagat gatcaatgag cagtttgaac cactgttgcc agaaggaaga | 600 |
| gatgtctttg atgtctgggg aaatgacagc aattacacaa agatcgtggc cgctgtagat | 660 |
| atgttcttcc atatgttcaa aaagcatgag aaggcctctt tcaggtatgg cacaatagtg | 720 |
| tcaagattta aggattgtgc agcattggct acatttggtc atctgtgtaa gatcactggt | 780 |
| atgtccactg aagatgtgac aacttggatt ctaaacaggg aggtggctga tgagatggtt | 840 |
| caaatgatgt acccaggaca ggagatagat aaggctgatt cttacatgcc ttatctaatc | 900 |
| gacttaggtc tgtcctcaaa atctccatat tcatcagtta aaaatccagc tttccatttt | 960 |
| tggggtcaat tgaccgcatt gttactgaga tcaaccagag ccagaaatgc acgtcagccg | 1020 |
| gatgacatcg agtatacatc cctgaccact gctgggctgt tgtatgcata tgccgttggt | 1080 |
| tcgtctgcag acctggctca acaattctac gttggggaca acaagtatgt gccagaaact | 1140 |
| ggagatggag gattaaccac caatgcaccg ccacaagggc gagatgtggt cgagtggctt | 1200 |
| agttggtttg aagatcaaaa cagaaaacct accccagaca tgctcatgta tgctaagaga | 1260 |
| gctgtcagtg ctttacaagg attgagggag aagacgattg gcaagtacgc caagtcagag | 1320 |
| tttgacaaat gacaactcac tcaccatatg tattactacc tttgcttcat atgaaaaaaa | 1380 |
| ctaacagcga tcatggatca gctatcaaag gtcaaggaat tccttaagac ttacgcgcag | 1440 |
| ttggatcaag cagtacaaga gatggatgac attgagtctc agagagagga aaagactaat | 1500 |
| tttgatttgt ttcaggaaga aggattggag attaaggaga agccttccta ttatcgggca | 1560 |
| gatgaagaag agattgattc agatgaagac agcgtggatg atgcacaaga cttagggata | 1620 |
| cgtacatcaa caagtcccat cgaggggtat gtggatgagg agcaggatga ttatgaggat | 1680 |
| gaggaagtga acgtggtgtt tacatcggac tggaaacagc ctgagctgga atccgacggg | 1740 |
| gatgggaaaa ctctccgatt gacgatacca gatggattga ctggggagca gaagtcgcaa | 1800 |
| tggcttgcca cgattaaggc agttgttcag agtgctaaat attggaacat ctcagaatgt | 1860 |
| tcatttgaga gttatgagca aggggttttg attagagaga gacaaatgac tcctgatgtc | 1920 |
| tacaaagtca ctcctgtttt aaatgctcca ccggttcaaa tgacagctaa tcaagatgtt | 1980 |
| tggtctctca gcagcactcc atttacattt ttgcccaaga aacaaggtgt gactccattg | 2040 |
| accatgtcct tagaagaacc cttcaacacc cgaggtgaat tcatatctct gggaggaaac | 2100 |
| gggaaaatga gtcaccggga ggccatcatt ctagggttga gacacaagaa gctctataat | 2160 |
| caagccagac taaagtataa cttagcttga atatgaaaaa aactaacaga tatcaaaaga | 2220 |
| tatctctaac tcagtccatt gtgttcagtt caatcatgag ctctctcaag aaaattttgg | 2280 |
| gtattaaagg gaaagggaag aaatctaaga aattaggtat ggctccccca ccctatgaag | 2340 |
| aagagactcc aatggaata* tctccaagtg caccttatga taagtcattg tttggagtcg | 2400 |
| aagatatgga tttccatgat caacgtcaac tccgatatga gaaatttcac ttctcattga | 2460 |
| agatgactgt gagatcaaac aaaccatttc gaaattatga tgacgttgca gcagcggtgt | 2520 |
| ccaattggga tcatatgtac atcggcatgg caggaaaacg tcctttttat aagatattag | 2580 |
| cattcatggg ttctactcta ttgaaggcta caccagccgt ctgggctgac caaggacagc | 2640 |
| cagaatatca tgctcactgt gagggacgag cttacttgcc gcatcggtta gggccgaccc | 2700 |
| ctccgatgtt gaatgtccct gaacattttc gccgtccatt taacatcgga ttattcagag | 2760 |
| ggacaatcga cataaccctg gtacttttcg atgatgaatc tgtagattct gccccggtca | 2820 |
| tatgggatca ttttaatgca tccagattga gcagcttcag agaaaaggct ttgttgtttg | 2880 |
| gtttgattct agaaaagaaa gccactggga attgggtatt ggactctatt agtcatttca | 2940 |
| agtaattatc acaagtgttg aggtgatggg cagactatga aaaaaactaa cagggttcaa | 3000 |
| acactcttga tcgaggtacc cagttatatt tgttacaaca atgttgagac tttttctctt | 3060 |
| ttgtttcttg gccttaggag cccactccaa atttactata gtattccctc atcatcaaaa | 3120 |
| agggaattgg aagaatgtgc cttccacata tcattattgc ccttctagtt ctgaccagaa | 3180 |
| ttggcataat gatttgactg gagttagtct tcatgtgaaa attcccaaaa gtcacaaagc | 3240 |
| tatacaagca gatggctgga tgtgccacgc tgctaaatgg gtgactactt gtgacttcag | 3300 |
| atggtacgga cccaaataca tcacgcattc catacactct atgtcaccca ccctagaaca | 3360 |
| gtgcaagacc agtattgagc agacaaagca aggagtttgg attaatccag gctttccccc | 3420 |
| tcaaagctgc ggatatgcta cagtgacgga tgcagaggtg gttgttgtac aagcaacacc | 3480 |
| tcatcatgtg ttggttgatg agtacacagg agaatggatt gactcacaat tggtgggggg | 3540 |
| caaatgttcc aagcaggttt gtcaaacggt tcacaactcg accgtgtggc atgctgatta | 3600 |
| caagattaca gggctgtgcg agtcaaatct ggcatcagtg gatatcacct tcttctctga | 3660 |
| ggatggtcaa aagacgtctt tgggaaaacc gaacactgga ttcaggagta atcactttgc | 3720 |
| ttacgaaagt ggagagaagg catgccgtat gcagtactgc acacgatggg gaatccgact | 3780 |
| accttctgga gtatggtttg aattagtgga caaagatctc ttccaggcgg caaaattgcc | 3840 |
| tgaatgtcct agaggatcca gtatctcagc tccttctcag acttctgtgg atgttagttt | 3900 |
| gatacaagac gtagagagga tcttagatta ctctctatgc caggagacgt ggagtaagat | 3960 |
| acgagccaag cttcctgtat ctccagtaga tctgagttat ctcgccccaa aaaatccagg | 4020 |
| gagcggaccg gccttcacta tcattaatgg cactttgaaa tatttcgaaa caagatacat | 4080 |
| cagagttgac ataagtaatc ccatcatccc tcacatggtg ggaacaatga gtggaaccac | 4140 |
| gactgagcgt gaattgtgga atgattggta tccatatgaa gacgtagaga ttggtccaaa | 4200 |
| tggggtgttg aaaactccca ctggtttcaa gtttccgctg tacatgattg ggcacggaat | 4260 |
| gttggattcc gatctccaca aatcctccca ggctcaagtc ttcgaacatc cacacgcaaa | 4320 |
| ggacgctgca tcacagcttc ctgatgatga gactttattt tttggtgaca caggactatc | 4380 |
| aaaaaaccca gtagagttag tagaaggctg gttcagtagc tggaagagca cattggcatc | 4440 |
| gttctttctg attataggct tgggggttgc attaatcttc atcattcgaa ttattgttgc | 4500 |
| gattcgctat aaatacaagg ggaggaagac ccaaaaaatt tacaatgatg tcgagatcag | 4560 |
| tcgattggga aataaataac agatgacgca tgagggtcag atcagattta cagcgtaagt | 4620 |
| gtgatattta ggattataaa ggttccttaa ttttaatttg ttacgcgttg tatgaaaaaa | 4680 |
| actcatcaac agccatcatg catcagaagc gaactgctat gtttcaggac cctcaggagc | 4740 |
| ggccacgcaa actgcctcag ctgtgcaccg aactgcagac aactatccac gacatcattc | 4800 |
| tggaatgcgt gtactgtaag cagcagctgc tgaggagaga ggtctatgac ttcgcttttc | 4860 |
| gcgatctgtg catcgtgtac cgagacggaa acccatatgc agtcgataag ctgaagttct | 4920 |
| acagcaagat ctccgaatac aggcattact gttacagcgt gtacgggacc acactggagc | 4980 |
| agcagtataa caagcccctg tgcgacctgc tgatcagaat taatcagaag cccctgtgcc | 5040 |
| ctgaggaaaa acagaggcac ctggataaga aacagagatt tcataacatc cgaggacgat | 5100 |
| ggaccgggcg gtgcatgtcc tgctgtagaa gctcccggac tcgacgagag acccagctgg | 5160 |
| gcggaggagg aggagcagct tacatggcac gattcgagga ccctacccga aggccatata | 5220 |
| agctgcccga cctgtgcaca gaactgaata cttctctgca ggacatcgag attacatgcg | 5280 |
| tgtactgtaa aaccgtcctg gagctgacag aagtgttcga gtttgctttc aaggacctgt | 5340 |
| ttgtggtcta ccgggattca atccctcacg cagcccataa aatcgacttc tacagcagga | 5400 |
| tcagggaact gcgccactac tccgacagcg tgtacgggga tacactggag aagctgacaa | 5460 |
| acactggcct gtacaatctg ctgatccgac tgcgacagaa gccactgaac ccagccgaaa | 5520 |
| aactgagaca cctgaacgag aagagacggt ttcacaatat tgcaggccat tataggggac | 5580 |
| agtgccatag ttgctgtaat cgagccaggc agcaaagact gcagcgccga agggagactc | 5640 |
| aagtcggcgg aggaggagga gctgcataca tgcacggcga cacccccaca ctgcatgaat | 5700 |
| atatgctgga tctgcagcct gagactaccg acctgzacca gctgaacgat tctagtgagg | 5760 |
| aagaggacga aatcgacgga ccagcaggac aggcagagcc tgaccgggcc cactataata | 5820 |
| ttgtgacatt ctgctgtaag tgcgattcta ctctgcggct gtgcgtgcag agtactcatg | 5880 |
| tcgacatccg caccctggag gatctgctga tggggactct gggcatcgtc ccaatttgta | 5940 |
| gccagaaacc aggcggcggc ggcggagcag cttacatgca cggacccaag gctaccctgc | 6000 |
| aggacatcgt gctgcatctg gaacctcaga atgagattcc agtcgacctg ctgcagctga | 6060 |
| gtgattcaga agaggaaaac gacgagatcg acggcgtgaa tcaccagcat ctgcctgcta | 6120 |
| gacgggcaga gccacagcga cacacaatgc tgtgcatgtg ctgtaagtgt gaagccagga | 6180 |
| tcaagctggt ggtcgagtca agcgccgacg atctgcgcgc cttccagcag ctgttcctga | 6240 |
| atactctgtc atttgtccct tggtgtgcct cccagcagtg acgtacgtgt atgaaaaaaa | 6300 |
| ctcatcaaca gccatcatgg atgttaacga ttttgagttg catgaggact ttgcattgtc | 6360 |
| tgaagatgac tttgtcactt cagaatttct caatccggaa gaccaaatga catacctgaa | 6420 |
| tcatgccgat tataatttga attctccctt aatcagcgat gatattgatt tcctgatcaa | 6480 |
| gaaatataat catgagcaaa ttccgaaaat gtgggatgtc aagaattggg agggagtgtt | 6540 |
| agagatgttg acagcctgtc aagccagtcc aattttatct agcactatgc ataagtgggt | 6600 |
| gggaaagtgg ctcatgtctg atgatcatga cgcaagccaa ggcttcagtt ttcttcatga | 6660 |
| agtggacaaa gaagctgatc tgacgtttga ggtggtggag acattcatta gaggatgggg | 6720 |
| aggtcgagaa ttgcagtaca agaggaaaga cacatttccg gactccttta gagttgcagc | 6780 |
| ctcattgtgt caaaaattcc ttgatttgca caaactcact ctgataatga attcagtctc | 6840 |
| tgaagtcgaa cttaccaacc tagcaaagaa ttttaaagga aaaaacagga aagcaaaaag | 6900 |
| cggaaatctg ataaccagat tgagggttcc cagtttaggt cctgcttttg tgactcaggc | 6960 |
| atgggtgtac atgaagaagt tggaaatgat tatggatcgg aattttttgt tgatgttgaa | 7020 |
| agacgttatc atcgggagga tgcagacgat cctgtccatg atctcaagag atgataatct | 7080 |
| cttctccgag tctgatatct ttactgtatt aaagatatac cggatagggg ataagatatt | 7140 |
| agaaaggcaa gggacaaagg gttacgactt gatcaaaatg attgagccta tttgtaactt | 7200 |
| aaagatgatg aatctggcac gtaaatatcg tcctctcatc cctacatttc ctcattttga | 7260 |
| aaaacatatt gctgactctg ttaaggaagg atcgaaaata gacaaaggga ttgagtttat | 7320 |
| atatgatcac attatgtcaa tccctggtgt ggacttgacc ttagttattt acggatcatt | 7380 |
| tcggcactgg ggtcatcctt ttatcaacta ctatgagggc ttagagaagc tacacaagca | 7440 |
| ggttacaatg cccaagacta ttgacagaga atatgcagaa tgtcttgcta gtgatctggc | 7500 |
| aagaatcgtt cttcagcaac aattcaatga acataagaaa tggtttgttg atgtagataa | 7560 |
| agtcccacaa tcccatcctt tcaaaagcca tatgaaagag aatacttggc ctactgcagc | 7620 |
| ccaagttcag gattacggcg atcgctggca tcagctccca ctcatcaaat gcttcgaaat | 7680 |
| cccagatttg ttagatccat cgatcatcta ctcagacaaa agtcattcca tgaaccggtc | 7740 |
| tgaagtacta cgacatgtaa gacttacacc tcatgtgccc attccaagca ggaaagtatt | 7800 |
| gcagacaatg ttggagacta aggcaacaga ctggaaagag tttttaaaga aaattgacga | 7860 |
| agaggggtta gaggatgatg atcttgtcat aggactcaaa gggaaagaga gagaattaaa | 7920 |
| aattgcggga agattctttt ctttgatgtc ctggaagctc agagagtatt ttgtcatcac | 7980 |
| tgagtatttg attaagacgc actttgtccc gatgtttaaa gggttgacca tggcggatga | 8040 |
| cttgacagcg gtgataaaga agatgatgga cacatcttca ggacaaggct tagataatta | 8100 |
| tgaatccatt tgtatagcca accatattga ctatgagaag tggaacaatc atcaaagaaa | 8160 |
| agagtcgaac gggcccgtgt tcaaggtgat gggtcaattc ttgggatatc cacgtctgat | 8220 |
| tgagagaact catgaatttt ttgagaagag tctgatatat tacaatggac gaccagatct | 8280 |
| gatgcgggtt cgaggaaatt ctctagtcaa cgcctcatct ttaaatgtct gctgggaggg | 8340 |
| tcaagctggg ggattagaag gactgcgaca gaagggatgg agtattctaa atttgcttgt | 8400 |
| cattcagaga gaagcaaaaa taaggaacac cgccgtgaaa gtgctagctc aaggtgacaa | 8460 |
| tcaggtgata tgtactcagt ataaaacgaa gaaatcccgg aatgatattg agcttaaggc | 8520 |
| agctctaaca cagatggtat ctaataatga gatgattatg tctgcgatta aatcaggcac | 8580 |
| cgagaaactg ggtcttttga ttaatgatga tgagacaatg caatctgctg attacctcaa | 8640 |
| ttacgggaag gttcccattt tcagaggagt aatcagaggc cttgagacaa aaagatggtc | 8700 |
| tcgagtgacc tgtgtgacaa atgatcagat tccaacgtgt gcgaacatta tgagctctgt | 8760 |
| gtcaactaat gcattaactg tagcccattt tgccgagaat ccagtcaatg ccatcattca | 8820 |
| gtataactac tttggaacat ttgcaaggct actgctgatg atgcatgacc ccgctctgag | 8880 |
| gatctctctg tatgaagtcc aatcaaaaat tccaggactt cacagtttga catttaaata | 8940 |
| ttctatgttg tatctggatc cttcgatagg aggagtctcc ggaatgtcac tctcgagatt | 9000 |
| cctcataaga tcatttccag atccagtgac agaaagtttg gcgttctgga aatttatcca | 9060 |
| ctctcatgca agaagcgatt cattaaagga gatatgtgca gtttttggaa atcctgaaat | 9120 |
| tgcaagattt cggctaactc atgtcgataa attggtggaa gacccaacct cattgaacat | 9180 |
| agctatggga atgagtcctg ctaatctatt aaagacagag gtaaaaaaat gtctactgga | 9240 |
| atcaaggcag agcatcaaga accagattgt aagagatgct actatttacc tacaccatga | 9300 |
| ggaagacaaa cttcgtagtt tcttatggtc cataacacca ctgttccctc ggttcttgag | 9360 |
| tgaattcaaa tctgggacat tcatcggagt agcagatggc ctgatcagct tatttcagaa | 9420 |
| ctctaggact attcgaaatt cttttaaaaa gcgttatcac agggaacttg atgatttaat | 9480 |
| aatcaagagc gaagtttcct cacttatgca tttgggtaag ctacatttga ggcgaggctc | 9540 |
| agttcgtatg tggacttgct cttctactca ggctgatctt ctccgattcc ggtcatgggg | 9600 |
| aagatctgtt ataggaacca cagtccctca tcccttagag atgttaggac aacattttaa | 9660 |
| aaaggagact ccttgcagtg cttgcaacat atccggatta gactatgtat ctgtccactg | 9720 |
| tccgaatggg attcatgacg tttttgaatc acgtggtcca ctccctgcat atttgggttc | 9780 |
| taaaacatcc gaatcaactt cgatcttgca gccgtgggag agagagagta aagtaccgtt | 9840 |
| gattaagcgt gccacaaggc ttcgtgatgc aatttcatgg tttgtgtctc ccgactctaa | 9900 |
| cttggcctca actatcctta agaacataaa tgcattaaca ggagaagaat ggtcaaagaa | 9960 |
| gcagcatgga tttaaaagga cgggatcggc gttacacagg ttctccacat ccaggatgag | 10020 |
| tcatggtggt tttgcttctc agagtacggc tgccttgact agattgatgg caactactga | 10080 |
| cactatgaga gatctgggag aacagaacta tgatttcctg tttcaggcga cattattcta | 10140 |
| tgctcaaata accacaactg tagtcaggaa tggatcattt catagctgca cggaccatta | 10200 |
| ocatataacc tgcaaatctt gtcsgagggc cattgatgag attaccttgg attcagcgat | 10260 |
| ggaatatagc cctccagatg tatcatcagt tttacaatct tggaggaatg gagaaggctc | 10320 |
| ttggggacat gaagtgaaac aaatataccc agttgaaggt gactggaggg gactatctcc | 10380 |
| tgttgaacaa tcttatcaag tcggacgctg tatcgggttt ctgttcggtg atctggcgta | 10440 |
| tagaaaatca tcccatgcag atgatagctc catgtttccg ttatctatac aaaacaaagt | 10500 |
| cagaggaaga ggctttttaa aagggcttat ggatgggtta atgagagcca gttgttgcca | 10560 |
| ggtgatccat cgtcgaagct tagcccatct gaagagaccg gctaatgcag tctatggagg | 10620 |
| gctgatttat ttgatagaca aattgagtgc atctgcccct tttctttcac tgacgagaca | 10680 |
| tggaccttta agggaagaat tagaaactgt tccacataag ataccgactt cttatcctac | 10740 |
| gagcaaccga gatatggggg tgatagttcg taattatttt aaatatcagt gcagactggt | 10800 |
| agaaaaaggt cggtacaaga cacattatcc tcaattgtgg cttttctcag atgtgctgtc | 10860 |
| cattgatttc ttaggacccc tgtctatatc ttcaactcta ttgggtattc tgtataaaca | 10920 |
| gacgttatct tctcgagaca aaaatgagtt gagagaactc gctaacttgt cttcattgtt | 10980 |
| gagatcagga gaaggatggg aagatatcca tgtcaaattc ttctctaagg acactttact | 11040 |
| ctgccctgaa gagatccgac atgcgtgcaa atttgggatt gctaaggaat ccgctgtttt | 11100 |
| aagctattat cctccttggt ctcaagagtc ttatggaggc atcacctcga tccccgtata | 11160 |
| tttttcgacc aggaagtatc ccaaaatttt agatgtccct cctcgggttc aaaacccatt | 11220 |
| ggtctcgggt ctacgattgg ggcaactccc tactggagca cattataaga ttaggagcat | 11280 |
| tgtaaagaac aagaaccttc gttatagaga tttccttagt tgtggggatg gatctggggg | 11340 |
| gatgaccgcg gcactattga gagaaaacag acaaagtagg ggaatcttca acagcctgtt | 11400 |
| agagttagcc ggatctctta tgagaggagc atctccagag cctccaagtg cactggagac | 11460 |
| gctcgggcaa gaacgatcta ggtgtgtgaa tggaagcaca tgttgggagt actcatctga | 11520 |
| cctaagccaa aaagagacat gggattactt cttaagattg aagagaggcc tgggtttgac | 11580 |
| cgtggactta atcaccatgg acatggaggt cagagaccct aatacaagtt tgatgataga | 11640 |
| aaagaacctc aaagtttatc tgcatcagat attagaacca actggtgtct taatatataa | 11700 |
| aacatacggg acccatattg cgacacaaac agataatatc ctgacgataa tcggtccttt | 11760 |
| ctttgagacg gttgacctag tccagtccga atacagcagc tcacaaacgt ccgaggtcta | 11820 |
| ttttgtagga cgaggcttgc gctctcatgt tgacgaaccc tgggtggact ggccatcctt | 11880 |
| aatggacaat tggagatcca tttatgcttt tcatgatcct actacagaat ttatcagagc | 11940 |
| aaaaaaagtc tgtgaaatug acagtcttat aggcattccg gctcaattca ttccagaccc | 12000 |
| atttgtaaat ctcgagacca tgctacagat agttggtgtt ccaacaggag tttcgcatgc | 12060 |
| cgcagctcta ttatcatcac aatatccaaa tcaattggtc acaacgtcaa tattttatat | 12120 |
| gacactcgtg tcttattata atgtaaacca tattcgaaga agccccaagc ctttctctcc | 12180 |
| tccgtctgat ggagtctcac agaacattgg ttcagccata gtcggactaa gtttttgggt | 12240 |
| gagtttgatg gagaatgatc tcggattata caaacaggct ctaggtgcaa taaagacgnc | 12300 |
| attccctatt agatggtcct ctgtccagac caaggatggg tttacacaag aatggagaac | 12360 |
| taaaggaaac ggaattccta aagattgtcg tctctcagac tctttggctc agataggaaa | 12420 |
| ctggatcaga gcgatggaat tggttaggaa caaaacgagg caatcaggat tttctgaaac | 12480 |
| cctatttgat caattctgcg gacttgcaga ccatcacctc aaatggcgga agttgggaaa | 12540 |
| cagaacagga attattgatt ggctaaataa tagaatttca tccattgaca aatccatctt | 12600 |
| ggtgaccaaa agtgatctgc atgacgagaa ctcatggagg gagtgaagat gtattcttcc | 12660 |
| acctctcatt gggtgatacc catatatgaa aaaaactata agtactttaa actctctttg | 12720 |
| ttttttaatg tatatctggt tttgttgttt ccgt | |

Protein sequence of an exemplary HPV E6/E7 fusion protein (SEQ ID NO: 5):
MHQKRTAMFQDPQERPRKLPQLCTELQTTIHDIILECVYCKQQLLRREVYDFAFRDLCIVYRDGNPYA
VDKLKFYSKISEYRHYCYSVYGTTLEQQYNKPLCDLLIRINQKPLCPEEKQRHLDKKQRFHNIRGRWT
GRCMSCCRSSRTRRETQLGGGGGAAYMARFEDPTRRPYKLPDLCTELNTSLQDIEITCVYCKTVLELT
EVFEFAFKDLFVVYRDSIPHAAHKIDFYSRIRELRHYSDSVYGDTLEKLTNTGLYNLLIRLRQKPLNP
AEKLRHLNEKRRFHNIAGHYRGQCHSCCNRARQERLQRRRETQVGGGGGAAYMHGPKATLQDIVLHLE
PQNEIPVDLLQLSDSEEENDEIDGVNHQHLPARRAEPQRHTMLCMCCKCEARIKLVVESSADDLRAFQ
QLFLNTLSFVPWCASQQGGGGGAAYMHGDTPTLHEYMLDLQPETTDLYQLNDSSEEEDEIDGPAGQAE
PDRAHYNIVTFCCKCDSTLRLCVQSTHVDIRTLEDLLMGTLGIVPICSQKP*
Protein sequence of an exemplary HPV E6/E7 fusion protein (SEQ ID NO: 6):
KARFEDPTRRPYKLPDLCTELNTSLQDIEITCVYCKTVLELTEVFEFAFKDLFVVYRDSIPHAAHKID
FYSRIRELRHYSDSVYGDTLEKLTNTGLYNLLIRLRQKPLNPAEKLRHLNEKRRFHNIAGHYRGQCHS
CCNRARQERLQRRRETQVGGGGGAAYMHQKRTAMFQDPQERPRKLPQLCTELQTTIHDIILECVYCKQ
QLLRREVYDFAFRDLCIVYRDGNPYAVDKLKFYSKISEYRHYCYSVYGTTLEQQYNKPLCDLLIRINQ
KPLCPEEKQRHLDKKQRFHNIRGRWTGRCMSCCRSSRTRRETQLGGGGGAAYMHGPKATLQDIVLHLE
PQNEIPVDLLQLSDSEEENDEIDGVNHQHLPARRAEPQRHTMLCMCCKCEARIKLVVESSADDLRAFQ
QLFLNTLSFVPWCASQQGGGGGAAYMHGDTPTLHEYMLDLQPETTDLYQLNDSSEEEDEIDGPAGQAE
PDRAHYNIVTFCCKCDSTLRLCVQSTHVDIRTLEDLLMGTLGIVPICSQKP*
Protein sequence of an exemplary HPV E6/E7 fusion protein (SEQ ID NO: 7):
MHGPKATLQDIVLHLEPQNEIPVDLLQLSDSEEENDEIDGVNHQHLPARRAEPQRHTMLCMCCKCEAR
IKLWESSADDLRAFQQLFLNTLSFVPWCASQQGGGGGAAYMHGDTPTLHEYMLDLQPETTDLYQLND
SSEEEDEIDGPAGQAEPDRAHYNIVTFCCKCDSTLRLCVQSTHVDIRTLEDLLMGTLGIVPICSQKPG
GGGGAAYMARFEDPTRRPYKLPDLCTELNTSLQDIEITCVYCKTVLELTEVFEFAFKDLFVVYRDSIP
HAAHKIDFYSRIRELRHYSDSVYGDTLEKLTNTGLYNLLIRLRQKPLNPAEKLRHLNEKRRFHNIAGH
YRGQCHSCCNRARQERLQRRRETQVGGGGGAAYMHQKRTAMFQDPQERPRKLPQLCTELQTTIHDIIL
ECVYCKQQLLRREVYDFAFRDLCIVYRDGNPYAVDKLKFYSKISEYRHYCYSVYGTTLEQQYNKPLCD
LLIRINQKPLCPEEKQRHLDKKQRFHNIRGRWTGRCMSCCRSSRTRRETQL*
Protein sequence of an exemplary HPV E6/E7 fusion protein (SEQ ID NO: 8):
MHQKRTAMFQDPQERPRKLPQLCTELQTTIHDIILECVYCKQQLLRREVYDFAFRDLCIVYRDGNPYA
VDKLKFYSKISEYRHYCYSVYGTTLEQQYNKPLCDLLIRINQKPLCPEEKQRHLDKKQRFHNIRGRWT
GRCMSCCRSSRTRRETQLGGGGGAAYMHGDTPTLHEYMLDLQPETTDLYQLNDSSEEEDEIDGPAGQA
EPDRAHYNIVTFCCKCDSTLRLCVQSTHVDIRTLEDLLMGTLGIVPICSQKPGGGGGAAYMHGPKATL
QDIVLHLEPQNEIPVDLLQLSDSEEENDEIDGVNHQHLPARRAEPQRHTMLCMCCKCEARIKLVVESS
ADDLRAFQQLFLNTLSFVPWCASQQGGGGGAAYMARFEDPTRRPYKLPDLCTELNTSLQDIEITCVYC
KTVLELTEVFEFAFKDLFVVYRDSIPHAAHKIDFYSRIRELRHYSDSVYGDTLEKLTNTGLYNLLIRL
RQKPLNPAF.KLRHLNEKRRFHNIAGHYRGQCHSCCNRARQERLQRRRETQV*
HPV16 E6 (SEQ ID NO: 9):
MHQKRTAMFQDPQERPRKLPQLCTELQTTIHDIILEXaaVYXaaKQQLLRREVYDFAFRDLCIVYRDG
NPYAVXaaDKXaaLKFYSKISEYRHYCYSLYGTTLEQQYNKPLCDLLIRXaaINXaaQKPLCPEEKQR
HLDKKQRFHNIRGRWTGRXaaMSXaaCRSSRTRRETQL
Note: when all Xaa's are cysteines, the sequence is the wild type
sequence
HPV18 E6 (SEQ ID NO: 10):
MARFEDPTRRPYKLPDLCTELNTSLQDIEITXaaVYXaaKTVLELTEVFEFAFKDLFVVYRDSIPHAA
XaaHKXaaIDFYSRIRELRHYSDSVYGDTLEKLTNTGLYNLLIRXaaLRXaaQKPLNPAEKLRHLNEK
RRFHNIAGHYRGQXaaHSXaaCNRARQERLQRRRETQV
Note: when all Xaa's are cysteines, the sequence is the wild type
sequence
HPV16 E7 (SEQ ID NO: 11):
MHGDTPTLHEYMLDLQPETTDLYXaaXaaXaaQLNDSSBEEDEIDGPAGQAEPDRAHYNIVTFCCKCD
STLRLCVQSTHVPIRTLEDLLMGTLGIVXaaPIXaaSQKP
Note: wild type sequence has XaaXaaxaa = CYE
Note: wild type sequence has cysteines at positions 91 and 94
HPV18E7 (SEQ ID NO: 12):
MHGPKATLQDIVLHLEPQNEIPVDLLXaaXaaXaaQLSDSEEENDEIDGVNHQHLPARRAEPQRHTML
CMCCKCBARIKLWESSAPDLRAFQQLFLNTLSFVXaaPWXaaASQQ
Note: wild type sequence has XaaXaaXaa = CHE
Note: wild type sequence has cysteines at positions 98 and 101
Protein sequence of an exemplary huSTEAP (SEQ ID NO: 13)
MESRKDITNQEELWKMKPRRNLEEDDYLHKDTGETSMLKRPVLLHLHQTAHADEFDCPSELQHTQELF
PQWHLPIKIAAIIASLTFLYTLLREVIHPLATSHQQYFYKIPILVINKVLPMVSITLLALVYLPGVIA
AIVQLHNGTKYKKFPHWLDKWMLTRKQFGLLSFFFAVLHAIYSLSYPMRRSYRYKLLNWAYQQVQQNK
EDAWIEHDVWRMEIYVSLGIVGLAILALLAVTSIPSVSDSLTWREFHYIQSKLGIVSLLLGTIHALIF
AWNKWIDIKQFVWYTPPTFMIAVFLPIVVLIFKSILFLPCLRKKILKIRHGWEDVTKINKTEICSQLK
L
DNA sequence of hu-STEAP protein (SEQ ID NO: 14):

| | |
|---|---|
| atggaatcac ggaaggacat cactaatcag gaggaactgt ggaaaatgaa gccaagaagg | 60 |
| aatctggaag aggacgacta tctgcacaag gacaccggcg aaacaagtat gctgaaacga | 120 |
| ccagtgctgc tgcacctgca tcagactgct cacgcagacg agtttgattg cccctctgaa | 180 |
| ctgcagcaca cccaggagct gttcccacag tggcatctgc ccatcaagat tgccgctatc | 240 |
| attgcttcac tgacatttct gtacactctg ctgagagaag tgatccaccc cctggccacc | 300 |
| agccatcagc agtacttcta taagatccct atcctggtca tcaacaaggt cctgccaatg | 360 |
| gtgagcatca cactgctggc cctggtctac ctgcctggag tgatcgcagc cattgtccag | 420 |
| ctgcacaatg ggacaaagta taagaaattt ccacattggc tggataagtg gatgctgact | 480 |
| aggaaacagt tcggactgct gtccttcttt ttcgccgtgc tgcacgctat ctacagcctg | 540 |
| tcctatccca tgaggaggag ctaccggtat aagctgctga actgggctta ccagcaggtg | 600 |
| cagcagaaca aggaggacgc atggattgaa catgacgtgt ggcgcatgga aatctacgcg | 660 |
| agcctgggca ttgccggact ggccatcctg gctctgctgg cagtgaccag tatcccttct | 720 |
| gtcagtgact caccgacatg gagagagttt cactacattc agagcaagct ggggatcgtg | 780 |
| tcccCgctgc tgggcaccat ccatgcactg atttttgcct ggaacaagcg gatcgatatc | 840 |
| aagcagttcg tgtggtatac tccccctacc tttatgattg ccgtcttcct gcccatcgtg | 900 |
| gtcctgatct tcaagtccat cctgttcctg ccttgtctgc ggaagaaaat cctgaaaatt | 960 |
| cggcacggat gggaggatgt caccaaaatc aataagactg aaatctgtag ccagctgaag | 1020 |
| ctttaa | 1026 |

Exemplary Maraba MG1 DNA sequence that encodes an huSTEAP protein (SEQ
ID NO: 15)

| | |
|---|---|
| acgaagacaa acaaaccatt gatagaatta agaggctcat gaaaatcctt aacagcgttc | 60 |
| aaaatgtctg ttacagtcaa gagagtcatt gatgattcac tcatcacccc caaattgcct | 120 |
| gcgaatgagg accctgtgga gcaccctgct gattatttca aaaagtcccg tgatattccg | 180 |
| gtgtacataa acacgaccaa aagtttgtct gatttgcggg gctatgttta tcaaggccta | 240 |
| aagtcaggca acatctctat aattcatgtc aacagttatc tgtatgcagc attaaaagag | 300 |
| atcagaggaa aattggacag agattggatc acctttggta tccaaatcgg aaaaacagga | 360 |
| gatagcgtgg ggatattcga tttactgacc ctaaaacctc tagatggtgt tttaccagat | 420 |
| ggggtgtctg atgctactcg aactagctca gacgatgcat ggcttccact gtatctattg | 480 |
| gggttataca gagttggtcg aacacagatg ccagaataca ggaagaagct gatggatggt | 540 |
| ctgattaatc aatgtaagat gatcaatgag cagtttgaac cactgttgcc agaaggaaga | 600 |
| gatgtctttg atgtctgggg aaatgacagc aattacacaa agattgtggc cgctgtagat | 660 |
| atgttcttcc atatgttcaa aaagcatgag aaggcctctt tcaggtatgg cacaatagtg | 720 |
| tcaagattta aggattgtgc agcattggct acatttggtc atctgtgtaa gatcactggt | 780 |
| atgtccactg aagatgtgac aacttggatt ctaaacaggg aggtggctga tgagatggtt | 840 |
| caaatgatgt acccaggaca ggagatagat aaggctgatt cttacatgcc ttatctaacc | 900 |
| gacttaggtc tgtcctcaaa atctccatat tcatcagtta aaaatccagc tttccatttt | 960 |
| tggggtcaat tgaccgcatt gttactgaga tcaaccagag ccagaaatgc acgtcagccg | 1020 |
| gatgacatcg agtatacatc cctgaccact gctgggctgt tgtatgcata tgccgttggt | 1080 |
| tcgtctgcag acctggctca acaattctac gttggggaca acaagtatgt gccagaaact | 1140 |
| ggagatggag gattaaccac caatgcaccg ccacaagggc gagatgtggt cgagtggctt | 1200 |
| agttggtttg aagatcaaaa cagaaaacct accccagaca tgctcatgta tgctaagaga | 1260 |
| gctgtcagtg ctttacaagg attgagggag aagacgattg gcaagtacgc caagtcagag | 1320 |
| tttgacaaat gacaactcac tcaccatatg tattactacc tttgcttcat atgaaaaaaa | 1380 |
| ctaacagcga tcatggatca gctatcaaag gtcaaggaat tccttaagac ttacgcgcag | 1440 |
| ttggatcaag cagtacaaga gatggatgac attgagtctc agagagagga aaagactaat | 1500 |
| tttgatttgt ttcaggaaga aggattggag attaaggaga agccttccta ttatcgggca | 1560 |
| gacgaagaag agattgattc agatgaagac agcgtggatg atgcacaaga cttagggata | 1620 |
| cgtacatcaa caagtcccat cgaggggtat gtggatgagg agcaggatga ttatgaggat | 1680 |
| gaggaagtga acgtggtgtt tacatcggac tggaaacagc ctgagctgga atccgacggg | 1740 |
| gatgggaaaa ctctccgatt gacgatacca gatggattga ctggggagca gaagtcgcaa | 1800 |
| tggcttgcca cgattaaggc agttgttcag agtgctaaat attggaacat ctcagaatgt | 1860 |
| tcatttgaga gttatgagca aggggttttg attagagaga gacaaatgac tcctgatgtc | 1920 |
| tacaaagtca ctcctgtttt aaatgctcca ccggttcaaa tgacagctaa tcaagatgtt | 1980 |
| tggtctctca gcagcactcc atttacattt ttgcccaaga aacaaggtgt gactccattg | 2040 |
| accatgtcct tagaagaact cttcaacacc cgaggtgaat tcatatctct gggaggaaac | 2100 |
| gggaaaatga gtcaccggga ggccatcatt ctagggttga gacacaagaa gctctataat | 2160 |
| caagccagac taaagtataa cttagcttga atatgaaaaa aactaacaga tatcaaaaga | 2220 |
| tatctctaac tcagtccatt gtgttcagtt caatcatgag ctctctcaag aaaattttgg | 2280 |
| gtattaaagg gaaagggaag aaatctaaga aattaggtat ggctccccca ccctatgaag | 2340 |
| aagagactcc aatggaatat tctccaagtg caccttatga taagtcattg tttggagtcg | 2400 |
| aagatatgga tttccatgat caacgtcaac tccgatatga gaaatttcac ttctcattca | 2460 |
| agatgactgt gagatcaaac aaaccatttc gaaattatga tgacgttgca gcagcggtct | 2520 |
| ccaattggga tcatatgtac atcggcatgg caggaaaacg tcctttttat aagatattag | 2580 |
| cattcatggg ttctactcta ttgaaggcta caccagccgt ctgggctgac caaggacagc | 2640 |
| cagaatatca tgctcactgt gagggacgag cttacttgcc gcatcggtta gggccgaccc | 2700 |
| ctccgatgtt gaatgtccct gaacattttc gccgtccatt taacatcgga ttattcagag | 2760 |
| ggacaatcga cataaccctg gtacttttcg atgatgaatc tgtagattct gccccggtca | 2820 |
| tatgggatca ttttaatgca tccagattga gcagcttcag agaaaaggct ttgttgtttg | 2880 |
| gtttgattct agaaaagaaa gccactggga attgggtatt ggactctatt agtcatttca | 2940 |
| agtaattatc acaagtgttg aggtgatggg cagactatga aaaaaactaa cagggttcaa | 3000 |
| acactcttga tcgaggtacc cagttatatt tgttacaaca atgttgagac tttttctctt | 3060 |
| ttgtttcttg gccttaggag cccactccaa atttactata gtattccctc atcatcaaaa | 3120 |
| agggaattgg aagaatgtgc cttccacata tcattattgc ccttctagtt ctgaccagaa | 3180 |
| ttggcataat gatttgactg gagttagtct tcatgtgaaa attcccaaaa gtcacaaagc | 3240 |
| tatacaagca gatggctgga tgtgccacgc tgctaaatgg gtgactactt gtgacttcag | 3300 |
| atggtacgga cccaaataca tcacgcattc catacactct atgtcaccca ccctagaaca | 3360 |
| gtgcaagacc agtattgagc agacaaagca aggagtttgg attaatccag gctttccccc | 3420 |
| tcaaagctgc ggatatgcta cagtgacgga tgcagaggtg gttgttgtac aagcaacacc | 3480 |
| tcatcatgtg ttggttgatg agtacacagg agaatggatt gactcacaat tggtgggggg | 3540 |
| caaatgttcc aaggaggttt gtcaaacggt tcacaactcg accgtgtggc atgctgatta | 3600 |
| caagattaca gggctgtgcg agtcaaatct ggcatcagtg gatatcacct tcttctctga | 3660 |
| ggatggtcaa aagacgtctt tgggaaaacc gaacactgga ttcaggagta atcactttgc | 3720 |
| ttacgaaagt ggagagaagg catgccgtat gcagtactgc acacgatggg gaatccgact | 3780 |
| accttctgga gtatggtttg aattagtgga caaagatctc ttccaggcgg caaaattgcc | 3840 |
| tgaatgtcct agaggatcca gtatctcagc tccttctcag acttctgtgg atgttagttt | 3900 |
| gatacaagac gtagagagga tcttagatta ctctctatgc caggagacgt ggagtaagat | 3960 |
| acgagccaag cttcctgtat ctccagtaga tctgagttat ctcgccccaa aaaatccagg | 4020 |
| gagcggaccg gccttcacta tcattaatgg cactttgaaa tatttcgaaa caagatacat | 4080 |
| cagagttgac ataagtaatc ccatcatccc tcacatggtg ggaacaatga gtggaaccac | 4140 |
| gactgagcgt gaattgtgga atgattggta tccatatgaa gacgtagaga ttggtccaaa | 4200 |
| tggggtgttg aaaactccca ctggtttcaa gtttccgctg tacatgattg ggcacggaat | 4260 |
| gttggattcc gatctccaca aatcctccca ggctcaagtc ttcgaacatc cacacgcaaa | 4320 |
| ggacgctgca tcacagcttc ctgatgatga gactttattt tttggtgaca caggactatc | 4380 |
| aaaaaaccca gtagagttag tagaaggctg gttcagtagc tggaagagca cattggcatc | 4440 |
| gttctttctg attataggct tgggggttgc attaatcttc atcattcgaa ttattgttgc | 4500 |
| gattcgctat aaatacaagg ggaggaagac ccaaaaaatt tacaatgatg tcgagatgag | 4560 |
| tcgattggga aataaataac agatgacgca tgagggtcag atcagattta cagcgtaagt | 4620 |
| gtgatattta ggattataaa ggttccttaa ttttaatttg ttacgcgttg tatgaaaaaa | 4680 |
| actcatcaac agccatcatg gaatcacgga aggacatcac taatcaggag gaactgtgga | 4740 |
| aaatgaagcc aagaaggaat ctggaagagg acgactatct gcacaaggac accggcgaaa | 4800 |
| caagtatgct gaaacgacca gtgctgctgc acctgcatca gactgctcac gcagacgagt | 4860 |
| ttgattgccc ctctgaactg cagcacaccc aggagctgtt cccacagtgg catctgccca | 4920 |
| tcaagattgc cgctatcatt gcttcactga catttctgta cactctgctg agagaagtga | 4980 |
| tccaccccct ggccaccagc catcagcagt acttctataa gatccctatc ctggtcatca | 5040 |
| acaaggtcct gccaatggtg agcatcacac tgctggccct ggtctacctg cctggagtga | 5100 |
| tcgcagccat tgtccagctg cacaatggga caaagtataa gaaatttcca cattggctgg | 5160 |
| ataagtggat gctgactagg aaacagttcg gactgctgtc cttctttttc gccgtgctgc | 5220 |
| acgctatcta cagcctgtcc tatcccatga ggaggagcta ccggtataag ctgctgaact | 5280 |
| gggcttacca gcaggtgcag cagaacaagg aggacgcatg gattgaacat gacgtgtggc | 5340 |
| gcatggaaat ctacgtgagc ctgggcattg tcggactggc catcctggct ctgctggcag | 5400 |
| tgaccagtat cccttctgtc agtgactcac tgacatggag agagtttcac tacattcaga | 5460 |
| gcaagctggg gatcgtgtcc ctgctgctgg gcaccatcca tgcactgatt tttgcctgga | 5520 |
| acaagtggat cgatatcaag cagttcgtgt ggtatactcc ccctaccttt atgattgccg | 5580 |
| tcttcctgcc catcgtggtc ctgatcttca agtccatcct gttcctgcct tgtctgcgga | 5640 |
| agaaaatcct gaaaattcgg cacggatggg aggatgtcac caaaatcaat aagactgaaa | 5700 |
| tctgtagcca gctgaagctt taacgtacgt gtatgaaaaa aactcatcaa cagccatcat | 5760 |
| ggatgttaac gattttgagt tgcatgagga ctttgcattg tctgaagatg actttgtcac | 5820 |
| ttcagaattt ctcaatccgg aagaccaaat gacatacctg aatcatgccg attataattt | 5880 |
| gaattctccc ttaatcagcg atgatattga tttcctgatc aagaaatata atcatgagca | 5940 |
| aattccgaaa atgtgggatg tcaagaattg ggagggagtg ttagagatgt tgacagcctg | 6000 |
| tcaagccagt ccaattttat ctagcactat gcataagtgg gtgggaaagt ggctcatgtc | 6060 |
| tgatgatcat gacgcaagcc aaggcttcag ttttcttcat gaagtggaca aagaagctga | 6120 |
| tctgacgttt gaggtggtgg agacattcat tagaggatgg ggaggtcgag aattgcagta | 6180 |
| caagaggaaa gacacatttc cggactccct tagagttgca gcctcattgt gtcaaaaatt | 6240 |
| ccttgatttg cacaaactca ctctgataat gaattcagtc tctgaagtcg aacttaccaa | 6300 |
| cctagcaaag aattttaaag gaaaaaacag gaaagcaaaa agcggaaatc tgataaccag | 6360 |
| attgagggtt cccagtttag gtcctgcttt tgtgactcag ggatgggtgt acatgaagaa | 6420 |
| gttggaaatg attatggatc ggaatttttt gttgatgttg aaagacgtta tcatcgggag | 6480 |
| gatgcagacg atcctgtcca tgatctcaag agatgataat ctcttctccg agtctgatat | 6540 |
| ctttactgta ttaaagatat accggatagg ggataagata ttagaaaggc aagggacaaa | 6600 |
| gggttacgac ttgatcaaaa tgattgagcc tatttgtaac ttaaagatga tgaatctggc | 6660 |
| acgtaaatat cgtcctctca tccctacatt tcctcatttt gaaaaacata ttgctgactc | 6720 |
| tgttaaggaa ggatcgaaaa tagacaaagg gattgagttt atatatgatc acattatgtc | 6780 |
| aatccctggt gtggacttga ccttagttat ttacggatca tttcggcact ggggtcatcc | 6840 |
| ttttatcaac tactatgagg gcttagagaa gctacacaag caggttacaa tgcccaagac | 6900 |
| tattgacaga gaatatgcag aatgtctcgc tagtgatctg gcaagaatcg ttcttcagca | 6960 |
| acaattcaat gaacataaga aatggtttgt tgatgtagat aaagtcccac aatcccatcc | 7020 |
| tttcaaaagc catatgaaag agaatacttg gcctactgca gcccaagttc aggattacgg | 7080 |
| cgatcgctgg catcagctcc cactcatcaa atgcttcgaa atcccagatt tgttagatcc | 7140 |
| atcgatcatc tactcagaca aaagtcattc catgaaccgg tctgaagtac tacgacatgt | 7200 |
| aagacttaca cctcatgtgc ccattccaag caggaaagta ttgcagacaa tgttggagac | 7260 |
| taaggcaaca gactggaaag agtttttaaa gaaaattgac gaagaggggt tagaggatga | 7320 |
| tgatcttgtc ataggactca aagggaaaga gagagaatta aaaattgcgg gaagattctt | 7380 |
| ttctttgatg tcctggaagc tcagagagta ttttgtcatc actgagtatt tgattaagac | 7440 |
| gcactttgtc ccgatgttta aagggttgac catggcggat gacttgacag cggtgataaa | 7500 |
| gaagatgatg gacacatctt caggacaagg cttagataat tatgaatcca tttgtatagc | 7560 |
| caaccatatt gactatgaga agtggaacaa tcatcaaaga aaagagtcga acgggcccgt | 7620 |
| gttcaaggtg atgggtcaat tcttgggata tccacgtctg attgagagaa ctcatgaatt | 7680 |
| ttttgagaag agtctgatat attacaatgg acgaccagat ctgatgcggg ttcgaggaaa | 7740 |
| ttctctagtc aacgcctcat ctttaaatgt ctgctgggag ggtcaagctg ggggattaga | 7800 |
| aggactgcga cagaagggat ggagtattct aaatttgctt gtcattcaga gagaagcaaa | 7860 |
| aataaggaac accgccgtga aagtgctagc tcaaggtgac aatcaggtga tatgtactca | 7920 |
| gtataaaacg aagaaatccc ggaatgatat tgagcttaag gcagctctaa cacagatggt | 7980 |
| atctaataat gagatgatta tgtctgcgat taaatcaggc accgagaaac tgggtctttt | 8040 |
| gattaatgat gatgagacaa tgcaatctgc tgattacctc aattacggga aggttcccat | 8100 |
| tttcagagga gtaatcagag gccttgagac aaaaagatgg tctcgagtga cctgtgtgac | 8160 |
| aaatgatcag attccaacgt gtgcgaacat tatgagctct gtgtcaacta atgcattaac | 8220 |
| tgtagcccat tttgccgaga atccagtcaa tgccatcatt cagtataact actttggaac | 8280 |
| atttgcaagg ctactgctga tgatgcatga ccccgctctg aggatctctc tgtatgaagt | 8340 |
| ccaatcaaaa attccaggac ttcacagttt gacatttaaa tattctatgt tgtatctgga | 8400 |
| tccttcgata ggaggagtct ccggaatgtc actctcgaga ttcctcataa gatcatttcc | 8460 |
| agatccagtg acagaaagtt tggcgttctg gaaatttatc cactctcatg caagaagcga | 8520 |
| ttcattaaag gagatatgtg cagtttttgg aaatcctgaa attgcaagat ttcggctaac | 8580 |
| tcatgtcgat aaattggtgg aagacccaac ctcattgaac atagctatgg gaatgagtcc | 8640 |
| tgctaatcta ttaaagacag aggtaaaaaa atgtctactg gaatcaaggc agagcatcaa | 8700 |
| gaaccagatt gcaagagatg ctactattta cctacaccat gaggaagaca aacttcgtag | 8760 |
| tttcttatgg tccataacac cactgttccc tcggttcttg agtgaattca aatctgggac | 8820 |
| attcatcgga gtagcagatg gcctgatcag cttatttcag aactctagga ctattcgaaa | 8880 |
| ttcttttaaa aagcgttatc acagggaact tgatgattta ataatcaaga gcgaagtttc | 8940 |
| ctcacttatg catttgggta agctacattt gaggcgaggc tcagttcgta tgtggacttg | 9000 |
| ctcttctact caggctgatc ttctccgatt ccggtcatgg ggaagatctg ttataggaac | 9060 |
| cacagtccct catcccttag agatgttagg acaacatttt aaaaaggaga ctccttgcag | 9120 |
| tgcttgcaac atatccggat tagactatgt atctgtccac tgtccgaatg ggattcatga | 9180 |
| cgtttttgaa tcacgtggtc cactccctgc atatttgggt tctaaaacat ccgaatcaac | 9240 |
| ttcgatcttg cagccgtggg agagagagag taaagtaccg ttgattaagc gtgccacaag | 9300 |
| gcttcgtgat gcaatttcat ggtttgtgtc tcccgactct aacttggcct caactatcct | 9360 |
| taagaacata aatgcattaa caggagaaga atggtcaaag aagcagcatg gatttaaaag | 9420 |
| gacgggatcg gcgttacaca ggttctccac atccaggatg agtcatggtg gttttgcttc | 9480 |
| tcagagtacg gctgccttga ctagattgat ggcaactact gacactatga gagatctggg | 9540 |
| agaacagaac tatgatttcc tgtttcaggc gacattattg tatgctcaaa taaccacaac | 9600 |
| tgtagtcagg aatggatcat ttcatagctg cacggaccat taccatataa cctgcaaatc | 9660 |
| ttgtctgagg gccattgatg agattacctt ggattcagcg atggaatata gccctccaga | 9720 |
| tgtatcatca gttttacaat cttggaggaa tggagaaggc tcttggggac atgaagtgaa | 9780 |
| acaaatatac ccagttgaag gtgactggag gggactatct cctgttgaac aatcttatca | 9840 |
| agtcggacgc tgtatcgggt ttctgttcgg tgatctggcg tatagaaaat catcccatgc | 9900 |
| agatgatagc tccacgtttc cgttatctat acaaaacaaa gtcagaggaa gaggcttttt | 9960 |
| aaaagggctt atggatgggt taatgagagc cagttgttgc caggtgatcc atcgtcgaag | 10020 |
| cttagcccat ctgaagagac cggctaatgc agtctatgga gggctgattt atttgataga | 10080 |
| caaattgagt gcatctgccc cttttctttc actgacgaga catggacctt taagggaaga | 10140 |
| attagaaact gttccacata agataccgac ttcttatcct acgagcaacc gagatatggg | 10200 |
| ggtgatagtt cgtaattatt ttaaatatca gtgcagactg gtagaaaaag gtcggtacaa | 10260 |
| gacacattat cctcaattgt ggcttttctc agatgtgctg tccattgatt tcttaggacc | 10320 |
| cctgtctata tcttcaactc tattgggtat tctgtataaa cagacgttat cttctcgaga | 10380 |
| caaaaatgag ttgagagaac tcgctaactt gtcttcattg ttgagatcag gagaaggatg | 10440 |
| ggaagatatc catgtcaaat tcttctctaa ggacacttta ctctgccctg aagagatccg | 10500 |
| acatgcgtgc aaatttggga ttgctaagga atccgctgtt ttaagctatt atcctccttg | 10560 |
| gtctcaagag tcttatggag gcatcacctc gatccccgta tatttttcga ccaggaagta | 10620 |
| tcccaaaatt ttagatgtcc ctcctcgggt tcaaaaccca ttggtctcgg gtctacgatt | 10680 |
| ggggcaactc cctactggag cacattataa gattaggagc attgtaaaga acaagaacct | 10740 |
| tcgttataga gatttcctta gttgtgggga tggatctggg gggatgaccg cggcactatt | 10800 |
| gagagaaaac agacaaagta ggggaanctt caacagcctg ttagagttag ccggatctct | 10860 |
| tatgagagga gcatctccag agcctccaag tgcactggag acgctcgggc aagaacgatc | 10920 |
| taggtgtgtg aatggaagca catgttggga gtactcatct gacctaagcc aaaaagagac | 10980 |
| atgggattac ttcttaagat tgaagagagg cctgggtttg accgtggact taatcaccat | 11040 |
| ggacatggag gtcagagacc ctaatacaag tttgatgata gaaaagaacc tcaaagttta | 11100 |
| tctgcatcag atattagaac caactggtgt cttaatatat aaaacatacg ggacccatat | 11160 |
| tgcgacacaa acagataata tcctgacgat aatcggtcct ttctttgaga cggttgacct | 11220 |
| agtccagtcc gaatacagca gctcacaaac gtccgaggtc tattttgtag gacgaggctt | 11280 |
| gcgctctcat gttgacgaac cctgggtgga ctggccatcc ttaatggaca attggagatc | 11340 |
| catttatgct tttcatgatc ctactacaga atttatcaga gcaaaaaaag tctgtgaaat | 11400 |
| tgacagtctt ataggcattc cggctcaatt cattccagac ccatttgtaa atctcgagac | 11460 |
| catgctacag atagttggtg ttccaacagg agtttcgcat gccgcagctc tattatcatc | 11520 |
| acaatatcca aatcaattgg tcacaacgtc aatattttat atgacactcg tgtcttatta | 11580 |
| taatgtaaac catattcgaa gaagccccaa gcctttctct cctccgtctg atggagtctc | 11640 |
| acagaacatt ggttcagcca tagtcggact aagtttttgg gtgagtttga tggagaatga | 11700 |
| tctcggatta tacaaacagg ctctaggtgc aataaagacg tcattcccta ttagatggtc | 11760 |
| ctctgtccag accaaggatg ggtttacaca agaatggaga actaaaggaa acggaattcc | 11820 |
| taaagattgt cgtctctcag actctttggc tcagatagga aactggatca gagcgatgga | 11880 |
| attggttagg aacaaaacga ggcaatcagg attttctgaa accctatttg atcaattctg | 11940 |
| cggacttgca gaccatcacc tcaaatggcg gaagttggga aacagaacag gaattattga | 1200C |
| ttggctaaat aatagaattt catccattga caaatccatc ttggtgacca aaagtgatct | 12060 |
| gcatgacgag aactcatgga gggagtgaag atgtattctt ccacctctca ttgggtgata | 12120 |
| cccatatatg aaaaaaacta taagtacttt aaactctctt tgttttttaa tgtatatctg | 12180 |
| gttttgttgt ttccgt | 12196 |

1-55. (canceled)

56. A method of treating a cancer in a mammal, said cancer being a tumour expressing an HPV protein, said method comprising:
a) administering a first virus, said first virus being an adenovirus capable of expressing an HPV tumour associated antigen, wherein said first virus is capable of generating immunity to said HPV tumour associated antigen; and
b) administering a second virus, said second virus being a Maraba MG1 virus comprising a nucleic acid capable of expressing an HPV tumour associated antigen, wherein said Maraba MG1 virus provides a therapeutic oncolytic effect in said mammal.

57. The method of claim 56, wherein the HPV tumour associated antigen comprises a HPV E6/E7 fusion protein.

58. The method of claim 57, wherein the HPV E6/E7 fusion protein comprises an amino acid sequence comprising the formula:
X1-L1-X2-L2-X3-L3-X4
wherein each of X1, X2, X3, and X4 is an epitope selected from the epitopes present in one of the following proteins: HPV16 E6, HPV16 E7, HPV18 E6, HPV18 E7; and
wherein L1, L2, and L3 can each be a linker or be absent.

59. The method of claim 59, wherein X1, X2, X3 and X4 comprise at least four different HPV epitopes.

60. The method of claim 58, wherein L1, L2 and L3 are proteosomal degradable linkers.

61. The method of claim 57, wherein the HPV E6/E7 fusion protein encoded by said first virus or said second virus comprises:
a) the HPV16 E6 protein with the amino acid sequence of SEQ ID NO: 9, or a variant thereof;
b) the HPV18 E6 protein with the amino acid sequence of SEQ ID NO: 10, or a variant thereof;
c) the HPV16 E7 protein with the amino acid sequence of SEQ ID NO: 11, or a variant thereof; or
d) the HPV18 E7 protein with the amino acid sequence of SEQ ID NO: 12, or a variant thereof.

62. The method of claim 61, wherein one or more of the Xaa's in SEQ ID NO: 9 are deleted or substituted for an amino acid that is not a cysteine.

63. The method of claims 61, wherein one or more of the Xaa's in SEQ ID NO: 10 are deleted or substituted for an amino acid that is not a cysteine.

64. The method of claim 61, wherein one or more of the Xaa's in SEQ ID NO: 11 are deleted or substituted for an amino acid that is not a cysteine, and wherein at least one of the following modifications are made:
a. the Xaa at position 24 of SEQ ID NO: 11 is either absent, or an amino acid that is not a cysteine;
b. the Xaa at position 25 of SEQ ID NO: 11 is either deleted or an amino acid that is not a tyrosine; or
c. the Xaa position at 26 of SEQ ID NO: 11 is either deleted or an amino acid that is not glutamic acid.

65. The method of claim 61, wherein one or more of the Xaa's in SEQ ID NO: 12 are deleted or substituted for an amino acid that is not a cysteine, and wherein at least one of the following modifications are made:
a. the Xaa at position 27 of SEQ ID NO: 12 is either absent, or an amino acid that is not a cysteine;
b. the Xaa at position 28 of SEQ ID NO: 12 is either deleted or an amino acid that is not a histidine; or
c. the Xaa position at 29 of SEQ ID NO: 12 is either deleted or an amino acid that is not glutamic acid.

66. The method of claim 56, wherein the HPV tumour associated antigen comprises an amino acid sequence that is at least 80% identical to SEQ ID NO: 1.

67. The method of claim 56, wherein the HPV tumour associated antigen comprises an amino acid sequence that is at least 80% identical to SEQ ID NO: 5.

68. The method of claim 56, wherein the HPV tumour associated antigen comprises an amino acid sequence that is at least 80% identical to SEQ ID NO: 6.

69. The method of claim 56, wherein the HPV tumour associated antigen comprises an amino acid sequence that is at least 80% identical to SEQ ID NO: 7.

70. The method of claim 56, wherein the HPV tumour associated antigen comprises an amino acid sequence that is at least 80% identical to SEQ ID NO: 8.

71. The method according to claim 56, wherein said Maraba MG1 virus is first administered at an interval of about 2 weeks after the said administration of said adenovirus.

72. The method according to claim 56, wherein the adenovirus is administered intramuscularly.

73. The method according to claim 56 wherein the Maraba MG1 virus is administered intravenously.

74. The method according to claim 56, wherein the cancer expresses an HPV tumour associated antigen.

75. The method according to claim 56, wherein said adenovirus and said Maraba MG1 virus comprise a nucleic acid capable of expressing an HPV tumour associated antigen that consists of an amino acid sequence that is SEQ ID NO: 1; wherein said adenovirus is administered intramuscularly and said Maraba MG1 virus is administered intravenously; wherein said Maraba MG1 virus is administered at least an interval of about two weeks after the administration of the adenovirus.

76. A heterologous prime-boost vaccine for use in the treatment of cancer in a mammal comprising:
a first virus, said first virus being an adenovirus comprising a nucleic acid capable of expressing an HPV tumour associated antigen, wherein said first virus is capable of generating immunity to said HPV tumour associated antigen; and
a second virus, said second virus being a Maraba MG1 virus comprising a nucleic acid capable of expressing a HPV tumour associated antigen, wherein said second virus is capable of providing a therapeutic oncolytic effect in said mammal.

77. The heterologous prime-boost vaccine of claim 76, wherein the HPV tumour associated antigen is a HPV E6/E7 fusion protein.

78. The heterologous prime-boost vaccine of claim 77, wherein the HPV E6/E7 fusion protein comprises the formula:
X1-L1-X2-L2-X3-L3-X4
wherein each of X1, X2, X3, and X4 is an epitope selected from the epitopes present in one of the following proteins: HPV16 E6, HPV16 E7, HPV18 E6, HPV18 E7; and
wherein L1, L2, and L3 can each be a linker or be absent.

79. The heterologous prime-boost vaccine of claim 78, wherein X1, X2, X3 and X4 comprise at least four different HPV epitopes.

80. The heterologous prime-boost vaccine of claim 78, wherein L1, L2 and L3 are proteosomal degradable linkers.

81. The heterologous prime-boost vaccine of claim 78, wherein the HPV E6/E7 fusion protein encoded by said first or said second virus comprises:
a. the HPV16 E6 protein with the amino acid sequence of SEQ ID NO: 9, or a variant thereof;
b. the HPV18 E6 protein with the amino acid sequence of SEQ ID NO: 10, or a variant thereof;
c. the HPV16 E7 protein with the amino acid sequence of SEQ ID NO: 11, or a variant thereof; and
d. the HPV18 E7 protein with the amino acid sequence of SEQ ID NO: 12, or a variant thereof.

82. The heterologous prime-boost vaccine of claim 81, wherein one or more of the Xaa's in SEQ ID NO: 9 are deleted or substituted for an amino acid that is not a cysteine.

83. The heterologous prime-boost vaccine of claim 81, wherein one or more of the Xaa's in SEQ ID NO: 10 are deleted or substituted for an amino acid that is not a cysteine.

84. The heterologous prime-boost vaccine of claim 81, wherein one or more of the Xaa's in SEQ ID NO: 11 are deleted or substituted for an amino acid that is not a cysteine, and wherein at least one of the following modifications are made:
a. the Xaa at position 24 of SEQ ID NO: 11 is either absent, or an amino acid that is not a cysteine;
b. the Xaa at position 25 of SEQ ID NO: 11 is either deleted or an amino acid that is not a tyrosine; or
c. the Xaa position at 26 of SEQ ID NO: 11 is either deleted or an amino acid that is not glutamic acid.

85. The heterologous prime-boost vaccine of claim 81, wherein one or more of the Xaa's in SEQ ID NO: 12 are deleted or substituted for an amino acid that is not a cysteine, and wherein at least one of the following modifications are made:
a. the Xaa at position 27 of SEQ ID NO: 12 is either absent, or an amino acid that is not a cysteine;
b. the Xaa at position 28 of SEQ ID NO: 12 is either deleted or an amino acid that is not a histidine; or
c. the Xaa position at 29 of SEQ ID NO: 12 is either deleted or an amino acid that is not glutamic acid.

86. The heterologous prime-boost vaccine of claim 76, wherein the HPV tumour associated antigen comprises an amino acid sequence that is at least 80% identical to SEQ ID NO: 1.

87. The heterologous prime-boost vaccine of claim 76, wherein the HPV tumour associated antigen comprises an amino acid sequence that is at least 80% identical to SEQ ID NO: 4.

88. The heterologous prime-boost vaccine of claim 76, wherein the HPV tumour associated antigen comprises an amino acid sequence that is at least 80% identical to SEQ ID NO: 5.

89. The heterologous prime-boost vaccine of claim 76, wherein the HPV tumour associated antigen comprises an amino acid sequence that is at least 80% identical to SEQ ID NO: 6.

90. The heterologous prime-boost vaccine of claim 76, wherein the HPV tumour associated antigen comprises an amino acid sequence that is at least 80% identical to SEQ ID NO: 7.

91. The heterologous prime-boost vaccine of claim 76, wherein the HPV tumour associated antigen comprises an amino acid sequence that is at least 80% identical to SEQ ID NO: 8.

92. The heterologous prime-boost vaccine of claim 76, wherein the HPV tumour associated antigen comprises an amino acid sequence that is encoded by the DNA sequence of SEQ ID NO: 3.

93. The heterologous prime-boost vaccine according to any one of claim 76, wherein said Maraba MG1 virus is first administered at an interval of about 2 weeks after the said administration of said adenovirus.

94. The heterologous prime-boost vaccine according to any one of claim 76, wherein the adenovirus is formulated for intramuscular administration.

95. The heterologous prime-boost vaccine according to any one of claim 76 wherein the Maraba MG1 boost is formulated for intravenous administration.

96. An HPV antigenic protein comprising an amino acid sequence that encodes:
a. the HPV16 E6 protein with the amino acid sequence of SEQ ID NO: 9, or a variant thereof;
b. the HPV18 E6 protein with the amino acid sequence of SEQ ID NO: 10, or a variant thereof;
c. the HPV16 E7 protein with the amino acid sequence of SEQ ID NO: 11, or a variant thereof; and
d. the HPV18 E7 protein with the amino acid sequence of SEQ ID NO: 12, or a variant thereof.

97. The antigenic protein of claim 96, wherein one or more of the Xaa's in SEQ ID NO: 9 are deleted or substituted for an amino acid that is not a cysteine.

98. The antigenic protein of claim 96, wherein one or more of the Xaa's in SEQ ID NO: 10 are deleted or substituted for an amino acid that is not a cysteine.

99. The antigenic protein of claim 96, wherein one or more of the Xaa's in SEQ ID NO: 11 are deleted or substituted for an amino acid that is not a cysteine, and wherein at least one of the following modifications are made:
a. the Xaa at position 24 of SEQ ID NO: 11 is either absent, or an amino acid that is not a cysteine;
b. the Xaa at position 25 of SEQ ID NO: 11 is either deleted or an amino acid that is not a tyrosine; or
c. the Xaa position at 26 of SEQ ID NO: 11 is either deleted or an amino acid that is not glutamic acid.

100. The antigenic protein of claim 96, wherein one or more of the Xaa's in SEQ ID NO: 12 are deleted or substituted for an amino acid that is not a cysteine, and wherein at least one of the following modifications are made:
a. the Xaa at position 27 of SEQ ID NO: 12 is either absent, or an amino acid that is not a cysteine;
b. the Xaa at position 28 of SEQ ID NO: 12 is either deleted or an amino acid that is not a histidine; or
c. the Xaa position at 29 of SEQ ID NO: 12 is either deleted or an amino acid that is not glutamic acid.

101. The antigenic protein of claim 96, wherein the HPV tumour associated antigen is at least 80% identical to SEQ ID NO: 1.

102. The antigenic protein of claim 96, wherein the HPV tumour associated antigen is at least 80% identical to SEQ ID NO: 4.

103. The antigenic protein of claim 96, wherein the HPV tumour associated antigen is at least 80% identical to SEQ ID NO: 5.

104. The antigenic protein of claim 96, wherein the HPV tumour associated antigen is at least 80% identical to SEQ ID NO: 6.

105. The antigenic protein of claim 96, wherein the HPV tumour associated antigen is at least 80% identical to SEQ ID NO: 7.

106. The antigenic protein of claim 96, wherein the HPV tumour associated antigen is at least 80% identical to SEQ ID NO: 8.

107. The heterologous prime-boost vaccine of claim 96, wherein the HPV tumour associated antigen that is expressed by said first virus, said Maraba MG1 virus, or both is encoded by the DNA sequence of SEQ ID NO: 3.


SEQUENCE LISTING (23 sequences)

SEQ ID NO: 1 - protein, 527 aa, Artificial Sequence
  Human Papilloma Virus (HPV) E6/E7 fusion protein
MHQKRTAMFQDPQERPRKLPQLCTELQTTIHDIILECVYCKQQLLRREVYDFAFRDLCIVYRDGNPYAVDKLKFYSKISEYRHYCYSVYGTTLEQQYNKPLCDLLIRINQKPLCPEEKQRHLDKKQRFHNIRGRWTGRCMSCCRSSRTRRETQLGGGGGAAYMARFEDPTRRPYKLPDLCTELNTSLQDIEITCVYCKTVLELTEVFEFAFKDLFVVYRDSIPHAAHKIDFYSRIRELRHYSDSVYGDTLEKLTNTGLYNLLIRLRQKPLNPAEKLRHLNEKRRFHNIAGHYRGQCHSCCNRARQERLQRRRETQVGGGGGAAYMHGDTPTLHEYMLDLQPETTDLYQLNDSSEEEDEIDGPAGQAEPDRAHYNIVTFCCKCDSTLRLCVQSTHVDIRTLEDLLMGTLGIVPICSQKPGGGGGAAYMHGPKATLQDIVLHLEPQNEIPVDLLQLSDSEEENDEIDGVNHQHLPARRAEPQRHTMLCMCCKCEARIKLVVESSADDLRAFQQLFLNTLSFVPWCASQQ

SEQ ID NO: 2 - protein, 8 aa, Artificial Sequence
  linker
GGGGGAAY

SEQ ID NO: 3 - DNA, 1584 bp, Artificial Sequence
  DNA sequence of HPV E6/E7 fusion protein
atgcatcagaagcgaactgctatgtttcaggaccctcaggagcggccacgcaaactgcctcagctgtgcaccgaactgcagacaactatccacgacatcattctggaatgcgtgtactgtaagcagcagctgctgaggagagaggtctatgacttcgcttttcgcgatctgtgcatcgtgtaccgagacggaaacccatatgcagtcgataagctgaagttctacagcaagatctccgaatacaggcattactgttacagcgtgtacgggaccacactggagcagcagtataacaagcccctgtgcgacctgctgatcagaattaatcagaagcccctgtgccctgaggaaaaacagaggcacctggataagaaacagagatttcataacatccgaggacgatggaccgggcggtgcatgtcctgctgtagaagctcccggactcgacgagagacccagctgggcggaggaggaggagcagcttacatggcacgattcgaggaccctacccgaaggccatataagctgcccgacctgtgcacagaactgaatacttctctgcaggacatcgagattacatgcgtgtactgtaaaaccgtcctggagctgacagaagtgttcgagtttgctttcaaggacctgtttgtggtctaccgggattcaatccctcacgcagcccataaaatcgacttctacagcaggatcagggaactgcgccactactccgacagcgtgtacggggatacactggagaagctgacaaacactggcctgtacaatctgctgatccgactgcgacagaagccactgaacccagccgaaaaactgagacacctgaacgagaagagacggtttcacaatattgcaggccattataggggacagtgccatagttgctgtaatcgagccaggcaggaaagactgcagcgccgaagggagactcaagtcggcggaggaggaggagctgcatacatgcacggcgacacccccacactgcatgaatatatgctggatctgcagcctgagactaccgacctgtaccagctgaacgattctagtgaggaagaggacgaaatcgacggaccagcaggacaggcagagcctgaccgggcccactataatattgtgacattctgctgtaagtgcgattctactctgcggctgtgcgtgcagagtactcatgtcgacatccgcaccctggaggatctgctgatggggactctgggcatcgtcccaatttgtagccagaaaccaggcggcggcggcggagcagcttacatgcacggacccaaggctaccctgcaggacatcgtgctgcatctggaacctcagaatgagattccagtcgacctgctgcagctgagtgattcagaagaggaaaacgacgagatcgacggcgtgaatcaccagcatctgcctgctagacgggcagagccacagcgacacacaatgctgtgcatgtgctgtaagtgtgaagccaggatcaagctggtggtcgagtcaagcgccgacgatctgcgcgccttccagcagctgttcctgaatactctgtcatttgtcccttggtgtgcctcccagcagtga

SEQ ID NO: 4 - DNA, 12754 bp, Artificial Sequence
  Maraba MG1 DNA sequence additionally encoding an HPV-E6E7 fusion protein
acgaagacaaacaaaccattgatagaattaagaggctcatgaaaatccttaacagcgttcaaaatgtctgttacagtcaagagagtcattgatgattcactcatcacccccaaattgcctgcgaatgaggaccctgtggagtaccctgctgattatttcaaaaagtcccgtgatattccggtgtacataaacacgaccaaaagtttgtctgatttgcggggctatgtttatcaaggcctaaagtcaggcaacatctctataattcatgtcaacagttatctgtatgcagcattaaaagagatcagaggaaaattggacagagattggatcacctttggtatccaaatcggaaaaacaggagatagcgtggggatattcgatttactgaccctaaaacctctagatggtgttttaccagatggggtgtctgatgctactcgaactagctcagacgatgcatggcttccactgtatctattggggttatacagagttggtcgaacacagatgccagaatacaggaagaagctgatggatggtctgattaatcaatgtaagatgatcaatgagcagtttgaaccactgttgccagaaggaagagatgtctttgatgtctggggaaatgacagcaattacacaaagattgtggccgctgtagatatgttcttccatatgttcaaaaagcatgagaaggcctctttcaggtatggcacaatagtgtcaagatttaaggattgtgcagcattggctacatttggtcatctgtgtaagatcactggtatgtccactgaagatgtgacaacttggattctaaacagggaggtggctgatgagatggttcaaatgatgtacccaggacaggagatagataaggctgattcttacatgccttatctaatcgacttaggtctgtcctcaaaatctccatattcatcagttaaaaatccagctttccatttttggggtcaattgaccgcattgttactgagatcaaccagagccagaaatgcacgtcagccggatgacatcgagtatacatccctgaccactgctgggctgttgtatgcatatgccgttggttcgtctgcagacctggctcaacaattctacgttggggacaacaagtatgtgccagaaactggagatggaggattaaccaccaatgcaccgccacaagggcgagatgtggtcgagtggcttagttggtttgaagatcaaaacagaaaacctaccccagacatgctcatgtatgctaagagagctgtcagtgctttacaaggattgagggagaagacgattggcaagtacgccaagtcagagtttgacaaatgacaactcactcaccatatgtattactacctttgcttcatatgaaaaaaactaacagcgatcatggatcagctatcaaaggtcaaggaattccttaagacttacgcgcagttggatcaagcagtacaagagatggatgacattgagtctcagagagaggaaaagactaattttgatttgtttcaggaagaaggattggagattaaggagaagccttcctattatcgggcagatgaagaagagattgattcagatgaagacagcgtggatgatgcacaagacttagggatacgtacatcaacaagtcccatcgaggggtatgtggatgaggagcaggatgattatgaggatgaggaagtgaacgtggtgtttacatcggactggaaacagcctgagctggaatccgacggggatgggaaaactctccgattgacgataccagatggattgactggggagcagaagtcgcaatggcttgccacgattaaggcagttgttcagagtgctaaatattggaacatctcagaatgttcatttgagagttatgagcaaggggttttgattagagagagacaaatgactcctgatgtctacaaagtcactcctgttttaaatgctccaccggttcaaatgacagctaatcaagatgtttggtctctcagcagcactccatttacatttttgcccaagaaacaaggtgtgactccattgaccatgtccttagaagaactcttcaacacccgaggtgaattcatatctctgggaggaaacgggaaaatgagtcaccgggaggccatcattctagggttgagacacaagaagctctataatcaagccagactaaagtataacttagcttgaatatgaaaaaaactaacagatatcaaaagatatctctaactcagtccattgtgttcagttcaatcatgagctctctcaagaaaattttgggtattaaagggaaagggaagaaatctaagaaattaggtatggctcccccaccctatgaagaagagactccaatggaatattctccaagtgcaccttatgataagtcattgtttggagtcgaagatatggatttccatgatcaacgtcaactccgatatgagaaatttcacttctcattgaagatgactgtgagatcaaacaaaccatttcgaaattatgatgacgttgcagcagcggtgtccaattgggatcatatgtacatcggcatggcaggaaaacgtcctttttataagatattagcattcatgggttctactctattgaaggctacaccagccgtctgggctgaccaaggacagccagaatatcatgctcactgtgagggacgagcttacttgccgcatcggttagggccgacccctccgatgttgaatgtccctgaacattttcgccgtccatttaacatcggattattcagagggacaatcgacataaccctggtacttttcgatgatgaatctgtagattctgccccggtcatatgggatcattttaatgcatccagattgagcagcttcagagaaaaggctttgttgtttggtttgattctagaaaagaaagccactgggaattgggtattggactctattagtcatttcaagtaattatcacaagtgttgaggtgatgggcagactatgaaaaaaactaacagggttcaaacactcttgatcgaggtacccagttatatttgttacaacaatgttgagactttttctcttttgtttcttggccttaggagcccactccaaatttactatagtattccctcatcatcaaaaagggaattggaagaatgtgccttccacatatcattattgcccttctagttctgaccagaattggcataatgatttgactggagttagtcttcatgtgaaaattcccaaaagtcacaaagctatacaagcagatggctggatgtgccacgctgctaaatgggtgactacttgtgacttcagatggtacggacccaaatacatcacgcattccatacactctatgtcacccaccctagaacagtgcaagaccagtattgagcagacaaagcaaggagtttggattaatccaggctttccccctcaaagctgcggatatgctacagtgacggatgcagaggtggttgttgtacaagcaacacctcatcatgtgttggttgatgagtacacaggagaatggattgactcacaattggtggggggcaaatgttccaaggaggtttgtcaaacggttcacaactcgaccgtgtggcatgctgattacaagattacagggctgtgcgagtcaaatctggcatcagtggatatcaccttcttctctgaggatggtcaaaagacgtctttgggaaaaccgaacactggattcaggagtaatcactttgcttacgaaagtggagagaaggcatgccgtatgcagtactgcacacgatggggaatccgactaccttctggagtatggtttgaattagtggacaaagatctcttccaggcggcaaaattgcctgaatgtcctagaggatccagtatctcagctccttctcagacttctgtggatgttagtttgatacaagacgtagagaggatcttagattactctctatgccaggagacgtggagtaagatacgagccaagcttcctgtatctccagtagatctgagttatctcgccccaaaaaatccagggagcggaccggccttcactatcattaatggcactttgaaatatttcgaaacaagatacatcagagttgacataagtaatcccatcatccctcacatggtgggaacaatgagtggaaccacgactgagcgtgaattgtggaatgattggtatccatatgaagacgtagagattggtccaaatggggtgttgaaaactcccactggtttcaagtttccgctgtacatgattgggcacggaatgttggattccgatctccacaaatcctcccaggctcaagtcttcgaacatccacacgcaaaggacgctgcatcacagcttcctgatgatgagactttattttttggtgacacaggactatcaaaaaacccagtagagttagtagaaggctggttcagtagctggaagagcacattggcatcgttctttctgattataggcttgggggttgcattaatcttcatcattcgaattattgttgcgattcgctataaatacaaggggaggaagacccaaaaaatttacaatgatgtcgagatgagtcgattgggaaataaataacagatgacgcatgagggtcagatcagatttacagcgtaagtgtgatatttaggattataaaggttccttaattttaatttgttacgcgttgtatgaaaaaaactcatcaacagccatcatgcatcagaagcgaactgctatgtttcaggaccctcaggagcggccacgcaaactgcctcagctgtgcaccgaactgcagacaactatccacgacatcattctggaatgcgtgtactgtaagcagcagctgctgaggagagaggtctatgacttcgcttttcgcgatctgtgcatcgtgtaccgagacggaaacccatatgcagtcgataagctgaagttctacagcaagatctccgaatacaggcattactgttacagcgtgtacgggaccacactggagcagcagtataacaagcccctgtgcgacctgctgatcagaattaatcagaagcccctgtgccctgaggaaaaacagaggcacctggataagaaacagagatttcataacatccgaggacgatggaccgggcggtgcatgtcctgctgtagaagctcccggactcgacgagagacccagctgggcggaggaggaggagcagcttacatggcacgattcgaggaccctacccgaaggccatataagctgcccgacctgtgcacagaactgaatacttctctgcaggacatcgagattacatgcgtgtactgtaaaaccgtcctggagctgacagaagtgttcgagtttgctttcaaggacctgtttgtggtctaccgggattcaatccctcacgcagcccataaaatcgacttctacagcaggatcagggaactgcgccactactccgacagcgtgtacggggatacactggagaagctgacaaacactggcctgtacaatctgctgatccgactgcgacagaagccactgaacccagccgaaaaactgagacacctgaacgagaagagacggtttcacaatattgcaggccattataggggacagtgccatagttgctgtaatcgagccaggcaggaaagactgcagcgccgaagggagactcaagtcggcggaggaggaggagctgcatacatgcacggcgacacccccacactgcatgaatatatgctggatctgcagcctgagactaccgacctgtaccagctgaacgattctagtgaggaagaggacgaaatcgacggaccagcaggacaggcagagcctgaccgggcccactataatattgtgacattctgctgtaagtgcgattctactctgcggctgtgcgtgcagagtactcatgtcgacatccgcaccctggaggatctgctgatggggactctgggcatcgtcccaatttgtagccagaaaccaggcggcggcggcggagcagcttacatgcacggacccaaggctaccctgcaggacatcgtgctgcatctggaacctcagaatgagattccagtcgacctgctgcagctgagtgattcagaagaggaaaacgacgagatcgacggcgtgaatcaccagcatctgcctgctagacgggcagagccacagcgacacacaatgctgtgcatgtgctgtaagtgtgaagccaggatcaagctggtggtcgagtcaagcgccgacgatctgcgcgccttccagcagctgttcctgaatactctgtcatttgtcccttggtgtgcctcccagcagtgacgtacgtgtatgaaaaaaactcatcaacagccatcatggatgttaacgattttgagttgcatgaggactttgcattgtctgaagatgactttgtcacttcagaatttctcaatccggaagaccaaatgacatacctgaatcatgccgattataatttgaattctcccttaatcagcgatgatattgatttcctgatcaagaaatataatcatgagcaaattccgaaaatgtgggatgtcaagaattgggagggagtgttagagatgttgacagcctgtcaagccagtccaattttatctagcactatgcataagtgggtgggaaagtggctcatgtctgatgatcatgacgcaagccaaggcttcagttttcttcatgaagtggacaaagaagctgatctgacgtttgaggtggtggagacattcattagaggatggggaggtcgagaattgcagtacaagaggaaagacacatttccggactcctttagagttgcagcctcattgtgtcaaaaattccttgatttgcacaaactcactctgataatgaattcagtctctgaagtcgaacttaccaacctagcaaagaattttaaaggaaaaaacaggaaagcaaaaagcggaaatctgataaccagattgagggttcccagtttaggtcctgcttttgtgactcagggatgggtgtacatgaagaagttggaaatgattatggatcggaattttttgttgatgttgaaagacgttatcatcgggaggatgcagacgatcctgtccatgatctcaagagatgataatctcttctccgagtctgatatctttactgtattaaagatataccggataggggataagatattagaaaggcaagggacaaagggttacgacttgatcaaaatgattgagcctatttgtaacttaaagatgatgaatctggcacgtaaatatcgtcctctcatccctacatttcctcattttgaaaaacatattgctgactctgttaaggaaggatcgaaaatagacaaagggattgagtttatatatgatcacattatgtcaatccctggtgtggacttgaccttagttatttacggatcatttcggcactggggtcatccttttatcaactactatgagggcttagagaagctacacaagcaggttacaatgcccaagactattgacagagaatatgcagaatgtcttgctagtgatctggcaagaatcgttcttcagcaacaattcaatgaacataagaaatggtttgttgatgtagataaagtcccacaatcccatcctttcaaaagccatatgaaagagaatacttggcctactgcagcccaagttcaggattacggcgatcgctggcatcagctcccactcatcaaatgcttcgaaatcccagatttgttagatccatcgatcatctactcagacaaaagtcattccatgaaccggtctgaagtactacgacatgtaagacttacacctcatgtgcccattccaagcaggaaagtattgcagacaatgttggagactaaggcaacagactggaaagagtttttaaagaaaattgacgaagaggggttagaggatgatgatcttgtcataggactcaaagggaaagagagagaattaaaaattgcgggaagattcttttctttgatgtcctggaagctcagagagtattttgtcatcactgagtatttgattaagacgcactttgtcccgatgtttaaagggttgaccatggcggatgacttgacagcggtgataaagaagatgatggacacatcttcaggacaaggcttagataattatgaatccatttgtatagccaaccatattgactatgagaagtggaacaatcatcaaagaaaagagtcgaacgggcccgtgttcaaggtgatgggtcaattcttgggatatccacgtctgattgagagaactcatgaattttttgagaagagtctgatatattacaatggacgaccagatctgatgcgggttcgaggaaattctctagtcaacgcctcatctttaaatgtctgctgggagggtcaagctgggggattagaaggactgcgacagaagggatggagtattctaaatttgcttgtcattcagagagaagcaaaaataaggaacaccgccgtgaaagtgctagctcaaggtgacaatcaggtgatatgtactcagtataaaacgaagaaatcccggaatgatattgagcttaaggcagctctaacacagatggtatctaataatgagatgattatgtctgcgattaaatcaggcaccgagaaactgggtcttttgattaatgatgatgagacaatgcaatctgctgattacctcaattacgggaaggttcccattttcagaggagtaatcagaggccttgagacaaaaagatggtctcgagtgacctgtgtgacaaatgatcagattccaacgtgtgcgaacattatgagctctgtgtcaactaatgcattaactgtagcccattttgccgagaatccagtcaatgccatcattcagtataactactttggaacatttgcaaggctactgctgatgatgcatgaccccgctctgaggatctctctgtatgaagtccaatcaaaaattccaggacttcacagtttgacatttaaatattctatgttgtatctggatccttcgataggaggagtctccggaatgtcactctcgagattcctcataagatcatttccagatccagtgacagaaagtttggcgttctggaaatttatccactctcatgcaagaagcgattcattaaaggagatatgtgcagtttttggaaatcctgaaattgcaagatttcggctaactcatgtcgataaattggtggaagacccaacctcattgaacatagctatgggaatgagtcctgctaatctattaaagacagaggtaaaaaaatgtctactggaatcaaggcagagcatcaagaaccagattgtaagagatgctactatttacctacaccatgaggaagacaaacttcgtagtttcttatggtccataacaccactgttccctcggttcttgagtgaattcaaatctgggacattcatcggagtagcagatggcctgatcagcttatttcagaactctaggactattcgaaattcttttaaaaagcgttatcacagggaacttgatgatttaataatcaagagcgaagtttcctcacttatgcatttgggtaagctacatttgaggcgaggctcagttcgtatgtggacttgctcttctactcaggctgatcttctccgattccggtcatggggaagatctgttataggaaccacagtccctcatcccttagagatgttaggacaacattttaaaaaggagactccttgcagtgcttgcaacatatccggattagactatgtatctgtccactgtccgaatgggattcatgacgtttttgaatcacgtggtccactccctgcatatttgggttctaaaacatccgaatcaacttcgatcttgcagccgtgggagagagagagtaaagtaccgttgattaagcgtgccacaaggcttcgtgatgcaatttcatggtttgtgtctcccgactctaacttggcctcaactatccttaagaacataaatgcattaacaggagaagaatggtcaaagaagcagcatggatttaaaaggacgggatcggcgttacacaggttctccacatccaggatgagtcatggtggttttgcttctcagagtacggctgccttgactagattgatggcaactactgacactatgagagatctgggagaacagaactatgatttcctgtttcaggcgacattattgtatgctcaaataaccacaactgtagtcaggaatggatcatttcatagctgcacggaccattaccatataacctgcaaatcttgtctgagggccattgatgagattaccttggattcagcgatggaatatagccctccagatgtatcatcagttttacaatcttggaggaatggagaaggctcttggggacatgaagtgaaacaaatatacccagttgaaggtgactggaggggactatctcctgttgaacaatcttatcaagtcggacgctgtatcgggtttctgttcggtgatctggcgtatagaaaatcatcccatgcagatgatagctccatgtttccgttatctatacaaaacaaagtcagaggaagaggctttttaaaagggcttatggatgggttaatgagagccagttgttgccaggtgatccatcgtcgaagcttagcccatctgaagagaccggctaatgcagtctatggagggctgatttatttgatagacaaattgagtgcatctgccccttttctttcactgacgagacatggacctttaagggaagaattagaaactgttccacataagataccgacttcttatcctacgagcaaccgagatatgggggtgatagttcgtaattattttaaatatcagtgcagactggtagaaaaaggtcggtacaagacacattatcctcaattgtggcttttctcagatgtgctgtccattgatttcttaggacccctgtctatatcttcaactctattgggtattctgtataaacagacgttatcttctcgagacaaaaatgagttgagagaactcgctaacttgtcttcattgttgagatcaggagaaggatgggaagatatccatgtcaaattcttctctaaggacactttactctgccctgaagagatccgacatgcgtgcaaatttgggattgctaaggaatccgctgttttaagctattatcctccttggtctcaagagtcttatggaggcatcacctcgatccccgtatatttttcgaccaggaagtatcccaaaattttagatgtccctcctcgggttcaaaacccattggtctcgggtctacgattggggcaactccctactggagcacattataagattaggagcattgtaaagaacaagaaccttcgttatagagatttccttagttgtggggatggatctggggggatgaccgcggcactattgagagaaaacagacaaagtaggggaatcttcaacagcctgttagagttagccggatctcttatgagaggagcatctccagagcctccaagtgcactggagacgctcgggcaagaacgatctaggtgtgtgaatggaagcacatgttgggagtactcatctgacctaagccaaaaagagacatgggattacttcttaagattgaagagaggcctgggtttgaccgtggacttaatcaccatggacatggaggtcagagaccctaatacaagtttgatgatagaaaagaacctcaaagtttatctgcatcagatattagaaccaactggtgtcttaatatataaaacatacgggacccatattgcgacacaaacagataatatcctgacgataatcggtcctttctttgagacggttgacctagtccagtccgaatacagcagctcacaaacgtccgaggtctattttgtaggacgaggcttgcgctctcatgttgacgaaccctgggtggactggccatccttaatggacaattggagatccatttatgcttttcatgatcctactacagaatttatcagagcaaaaaaagtctgtgaaattgacagtcttataggcattccggctcaattcattccagacccatttgtaaatctcgagaccatgctacagatagttggtgttccaacaggagtttcgcatgccgcagctctattatcatcacaatatccaaatcaattggtcacaacgtcaatattttatatgacactcgtgtcttattataatgtaaaccatattcgaagaagccccaagcctttctctcctccgtctgatggagtctcacagaacattggttcagccatagtcggactaagtttttgggtgagtttgatggagaatgatctcggattatacaaacaggctctaggtgcaataaagacgtcattccctattagatggtcctctgtccagaccaaggatgggtttacacaagaatggagaactaaaggaaacggaattcctaaagattgtcgtctctcagactctttggctcagataggaaactggatcagagcgatggaattggttaggaacaaaacgaggcaatcaggattttctgaaaccctatttgatcaattctgcggacttgcagaccatcacctcaaatggcggaagttgggaaacagaacaggaattattgattggctaaataatagaatttcatccattgacaaatccatcttggtgaccaaaagtgatctgcatgacgagaactcatggagggagtgaagatgtattcttccacctctcattgggtgatacccatatatgaaaaaaactataagtactttaaactctctttgttttttaatgtatatctggttttgttgtttccgt

SEQ ID NO: 5 - protein, 527 aa, Artificial Sequence
  Exemplary HPV E6/E7 fusion protein
MHQKRTAMFQDPQERPRKLPQLCTELQTTIHDIILECVYCKQQLLRREVYDFAFRDLCIVYRDGNPYAVDKLKFYSKISEYRHYCYSVYGTTLEQQYNKPLCDLLIRINQKPLCPEEKQRHLDKKQRFHNIRGRWTGRCMSCCRSSRTRRETQLGGGGGAAYMARFEDPTRRPYKLPDLCTELNTSLQDIEITCVYCKTVLELTEVFEFAFKDLFVVYRDSIPHAAHKIDFYSRIRELRHYSDSVYGDTLEKLTNTGLYNLLIRLRQKPLNPAEKLRHLNEKRRFHNIAGHYRGQCHSCCNRARQERLQRRRETQVGGGGGAAYMHGPKATLQDIVLHLEPQNEIPVDLLQLSDSEEENDEIDGVNHQHLPARRAEPQRHTMLCMCCKCEARIKLVVESSADDLRAFQQLFLNTLSFVPWCASQQGGGGGAAYMHGDTPTLHEYMLDLQPETTDLYQLNDSSEEEDEIDGPAGQAEPDRAHYNIVTFCCKCDSTLRLCVQSTHVDIRTLEDLLMGTLGIVPICSQKP

SEQ ID NO: 6 - protein, 527 aa, Artificial Sequence
  Exemplary HPV E6/E7 fusion protein
MARFEDPTRRPYKLPDLCTELNTSLQDIEITCVYCKTVLELTEVFEFAFKDLFVVYRDSIPHAAHKIDFYSRIRELRHYSDSVYGDTLEKLTNTGLYNLLIRLRQKPLNPAEKLRHLNEKRRFHNIAGHYRGQCHSCCNRARQERLQRRRETQVGGGGGAAYMHQKRTAMFQDPQERPRKLPQLCTELQTTIHDIILECVYCKQQLLRREVYDFAFRDLCIVYRDGNPYAVDKLKFYSKISEYRHYCYSVYGTTLEQQYNKPLCDLLIRINQKPLCPEEKQRHLDKKQRFHNIRGRWTGRCMSCCRSSRTRRETQLGGGGGAAYMHGPKATLQDIVLHLEPQNEIPVDLLQLSDSEEENDEIDGVNHQHLPARRAEPQRHTMLCMCCKCEARIKLVVESSADDLRAFQQLFLNTLSFVPWCASQQGGGGGAAYMHGDTPTLHEYMLDLQPETTDLYQLNDSSEEEDEIDGPAGQAEPDRAHYNIVTFCCKCDSTLRLCVQSTHVDIRTLEDLLMGTLGIVPICSQKP

SEQ ID NO: 7 - protein, 527 aa, Artificial Sequence
  Exemplary HPV E6/E7 fusion protein
MHGPKATLQDIVLHLEPQNEIPVDLLQLSDSEEENDEIDGVNHQHLPARRAEPQRHTMLCMCCKCEARIKLVVESSADDLRAFQQLFLNTLSFVPWCASQQGGGGGAAYMHGDTPTLHEYMLDLQPETTDLYQLNDSSEEEDEIDGPAGQAEPDRAHYNIVTFCCKCDSTLRLCVQSTHVDIRTLEDLLMGTLGIVPICSQKPGGGGGAAYMARFEDPTRRPYKLPDLCTELNTSLQDIEITCVYCKTVLELTEVFEFAFKDLFVVYRDSIPHAAHKIDFYSRIRELRHYSDSVYGDTLEKLTNTGLYNLLIRLRQKPLNPAEKLRHLNEKRRFHNIAGHYRGQCHSCCNRARQERLQRRRETQVGGGGGAAYMHQKRTAMFQDPQERPRKLPQLCTELQTTIHDIILECVYCKQQLLRREVYDFAFRDLCIVYRDGNPYAVDKLKFYSKISEYRHYCYSVYGTTLEQQYNKPLCDLLIRINQKPLCPEEKQRHLDKKQRFHNIRGRWTGRCMSCCRSSRTRRETQL

SEQ ID NO: 8 - protein, 527 aa, Artificial Sequence
  Exemplary HPV E6/E7 fusion protein
MHQKRTAMFQDPQERPRKLPQLCTELQTTIHDIILECVYCKQQLLRREVYDFAFRDLCIVYRDGNPYAVDKLKFYSKISEYRHYCYSVYGTTLEQQYNKPLCDLLIRINQKPLCPEEKQRHLDKKQRFHNIRGRWTGRCMSCCRSSRTRRETQLGGGGGAAYMHGDTPTLHEYMLDLQPETTDLYQLNDSSEEEDEIDGPAGQAEPDRAHYNIVTFCCKCDSTLRLCVQSTHVDIRTLEDLLMGTLGIVPICSQKPGGGGGAAYMHGPKATLQDIVLHLEPQNEIPVDLLQLSDSEEENDEIDGVNHQHLPARRAEPQRHTMLCMCCKCEARIKLVVESSADDLRAFQQLFLNTLSFVPWCASQQGGGGGAAYMARFEDPTRRPYKLPDLCTELNTSLQDIEITCVYCKTVLELTEVFEFAFKDLFVVYRDSIPHAAHKIDFYSRIRELRHYSDSVYGDTLEKLTNTGLYNLLIRLRQKPLNPAEKLRHLNEKRRFHNIAGHYRGQCHSCCNRARQERLQRRRETQV

SEQ ID NO: 9 - protein, 158 aa, Artificial Sequence
  Artificial HPV16 E6 protein sequence. When all Xaa′s are cystines, the sequence corresponds to the wildtype HPV16 E6 protein sequence.
  MISC_FEATURE (37)..(37): present or absent, if present Xaa can be any naturally occurring amino acid
  MISC_FEATURE (40)..(40): present or absent, if present Xaa can be any naturally occurring amino acid
  MISC_FEATURE (70)..(70): present or absent, if present Xaa can be any naturally occurring amino acid
  MISC_FEATURE (73)..(73): present or absent, if present Xaa can be any naturally occurring amino acid
  MISC_FEATURE (110)..(110): present or absent, if present Xaa can be any naturally occurring amino acid
  MISC_FEATURE (113)..(113): present or absent, if present Xaa can be any naturally occurring amino acid
  MISC_FEATURE (143)..(143): present or absent, if present Xaa can be any naturally occurring amino acid
  MISC_FEATURE (146)..(146): present or absent, if present Xaa can be any naturally occurring amino acid
  MISC_FEATURE (146)..(146): Xaa may be present or absent, if present Xaa can be any naturally occurring amino acid
MHQKRTAMFQDPQERPRKLPQLCTELQTTIHDIILEXVYXKQQLLRREVYDFAFRDLCIVYRDGNPYAVXDKXLKFYSKISEYRHYCYSLYGTTLEQQYNKPLCDLLIRXINXQKPLCPEEKQRHLDKKQRFHNIRGRWTGRXMSXCRSSRTRRETQL

SEQ ID NO: 10 - protein, 158 aa, Artificial Sequence
  Artificial HPV18 E6 protein sequence. When all Xaa′s are cystines, the sequence corresponds to the wildtype HPV18 E6 protein sequence.
  MISC_FEATURE (32)..(32): Xaa may be present or absent, if present Xaa can be any naturally occurring amino acid
  MISC_FEATURE (35)..(35): Xaa may be present or absent, if present Xaa can be any naturally occurring amino acid
  MISC_FEATURE (65)..(65): Xaa may be present or absent, if present Xaa can be any naturally occurring amino acid
  MISC_FEATURE (68)..(68): Xaa may be present or absent, if present Xaa can be any naturally occurring amino acid
  MISC_FEATURE (105)..(105): Xaa may be present or absent, if present Xaa can be any naturally occurring amino acid
  MISC_FEATURE (108)..(108): Xaa may be present or absent, if present Xaa can be any naturally occurring amino acid
  MISC_FEATURE (138)..(138): Xaa may be present or absent, if present Xaa can be any naturally occurring amino acid
  MISC_FEATURE (141)..(141): Xaa may be present or absent, if present Xaa can be any naturally occurring amino acid
MARFEDPTRRPYKLPDLCTELNTSLQDIEITXVYXKTVLELTEVFEFAFKDLFVVYRDSIPHAAXHKXIDFYSRIRELRHYSDSVYGDTLEKLTNTGLYNLLIRXLRXQKPLNPAEKLRHLNEKRRFHNIAGHYRGQXHSXCNRARQERLQRRRETQV

SEQ ID NO: 11 - protein, 98 aa, Artificial Sequence
  Artificial HPV16 E7 protein sequence. When XaaXaaXaa is CYE and Xaa′s at positions 91 and 94 are cystein sequence corresponds to the wildtype HPV16 E7 protein sequence.
  MISC_FEATURE (24)..(26): Xaa′s may be present or absent, if present Xaa can be any naturally occurring amino acid
  MISC_FEATURE (91)..(91): Xaa may be present or absent, if present Xaa can be any naturally occurring amino acid
  MISC_FEATURE (94)..(94): Xaa may be present or absent, if present Xaa can be any naturally occurring amino acid
MHGDTPTLHEYMLDLQPETTDLYXXXQLNDSSEEEDEIDGPAGQAEPDRAHYNIVTFCCKCDSTLRLCVQSTHVDIRTLEDLLMGTLGIVXPIXSQKP

SEQ ID NO: 12 - protein, 105 aa, Artificial Sequence
  Artificial HPV18 E7 protein sequence. When XaaXaaXaa is CHE and Xaa′s at positions 98 and 101 are cystein sequence corresponds to the wildtype HPV18 E7 protein sequence.
  MISC_FEATURE (27)..(29): Xaa′s may be present or absent, if present Xaa can be any naturally occurring amino acid
  MISC_FEATURE (98)..(98): Xaa may be present or absent, if present Xaa can be any naturally occurring amino acid
  MISC_FEATURE (101)..(101): Xaa may be present or absent, if present Xaa can be any naturally occurring amino acid
MHGPKATLQDIVLHLEPQNEIPVDLLXXXQLSDSEEENDEIDGVNHQHLPARRAEPQRHTMLCMCCKCEARIKLVVESSADDLRAFQQLFLNTLSFVXPWXASQQ

SEQ ID NO: 13 - protein, 341 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQLKL

SEQ ID NO: 14 - DNA, 1026 bp, Artificial Sequence
  Codon optimized DNA sequence for huSTEAP protein
atggaatcacggaaggacatcactaatcaggaggaactgtggaaaatgaagccaagaaggaatctggaagaggacgactatctgcacaaggacaccggcgaaacaagtatgctgaaacgaccagtgctgctgcacctgcatcagactgctcacgcagacgagtttgattgcccctctgaactgcagcacacccaggagctgttcccacagtggcatctgcccatcaagattgccgctatcattgcttcactgacatttctgtacactctgctgagagaagtgatccaccccctggccaccagccatcagcagtacttctataagatccctatcctggtcatcaacaaggtcctgccaatggtgagcatcacactgctggccctggtctacctgcctggagtgatcgcagccattgtccagctgcacaatgggacaaagtataagaaatttccacattggctggataagtggatgctgactaggaaacagttcggactgctgtccttctttttcgccgtgctgcacgctatctacagcctgtcctatcccatgaggaggagctaccggtataagctgctgaactgggcttaccagcaggtgcagcagaacaaggaggacgcatggattgaacatgacgtgtggcgcatggaaatctacgtgagcctgggcattgtcggactggccatcctggctctgctggcagtgaccagtatcccttctgtcagtgactcactgacatggagagagtttcactacattcagagcaagctggggatcgtgtccctgctgctgggcaccatccatgcactgatttttgcctggaacaagtggatcgatatcaagcagttcgtgtggtatactccccctacctttatgattgccgtcttcctgcccatcgtggtcctgatcttcaagtccatcctgttcctgccttgtctgcggaagaaaatcctgaaaattcggcacggatgggaggatgtcaccaaaatcaataagactgaaatctgtagccagctgaagctttaa

SEQ ID NO: 15 - DNA, 12196 bp, Artificial Sequence
  DNA sequence of Maraba MG1 virus endocing huSTEAP protein
acgaagacaaacaaaccattgatagaattaagaggctcatgaaaatccttaacagcgttcaaaatgtctgttacagtcaagagagtcattgatgattcactcatcacccccaaattgcctgcgaatgaggaccctgtggagtaccctgctgattatttcaaaaagtcccgtgatattccggtgtacataaacacgaccaaaagtttgtctgatttgcggggctatgtttatcaaggcctaaagtcaggcaacatctctataattcatgtcaacagttatctgtatgcagcattaaaagagatcagaggaaaattggacagagattggatcacctttggtatccaaatcggaaaaacaggagatagcgtggggatattcgatttactgaccctaaaacctctagatggtgttttaccagatggggtgtctgatgctactcgaactagctcagacgatgcatggcttccactgtatctattggggttatacagagttggtcgaacacagatgccagaatacaggaagaagctgatggatggtctgattaatcaatgtaagatgatcaatgagcagtttgaaccactgttgccagaaggaagagatgtctttgatgtctggggaaatgacagcaattacacaaagattgtggccgctgtagatatgttcttccatatgttcaaaaagcatgagaaggcctctttcaggtatggcacaatagtgtcaagatttaaggattgtgcagcattggctacatttggtcatctgtgtaagatcactggtatgtccactgaagatgtgacaacttggattctaaacagggaggtggctgatgagatggttcaaatgatgtacccaggacaggagatagataaggctgattcttacatgccttatctaatcgacttaggtctgtcctcaaaatctccatattcatcagttaaaaatccagctttccatttttggggtcaattgaccgcattgttactgagatcaaccagagccagaaatgcacgtcagccggatgacatcgagtatacatccctgaccactgctgggctgttgtatgcatatgccgttggttcgtctgcagacctggctcaacaattctacgttggggacaacaagtatgtgccagaaactggagatggaggattaaccaccaatgcaccgccacaagggcgagatgtggtcgagtggcttagttggtttgaagatcaaaacagaaaacctaccccagacatgctcatgtatgctaagagagctgtcagtgctttacaaggattgagggagaagacgattggcaagtacgccaagtcagagtttgacaaatgacaactcactcaccatatgtattactacctttgcttcatatgaaaaaaactaacagcgatcatggatcagctatcaaaggtcaaggaattccttaagacttacgcgcagttggatcaagcagtacaagagatggatgacattgagtctcagagagaggaaaagactaattttgatttgtttcaggaagaaggattggagattaaggagaagccttcctattatcgggcagatgaagaagagattgattcagatgaagacagcgtggatgatgcacaagacttagggatacgtacatcaacaagtcccatcgaggggtatgtggatgaggagcaggatgattatgaggatgaggaagtgaacgtggtgtttacatcggactggaaacagcctgagctggaatccgacggggatgggaaaactctccgattgacgataccagatggattgactggggagcagaagtcgcaatggcttgccacgattaaggcagttgttcagagtgctaaatattggaacatctcagaatgttcatttgagagttatgagcaaggggttttgattagagagagacaaatgactcctgatgtctacaaagtcactcctgttttaaatgctccaccggttcaaatgacagctaatcaagatgtttggtctctcagcagcactccatttacatttttgcccaagaaacaaggtgtgactccattgaccatgtccttagaagaactcttcaacacccgaggtgaattcatatctctgggaggaaacgggaaaatgagtcaccgggaggccatcattctagggttgagacacaagaagctctataatcaagccagactaaagtataacttagcttgaatatgaaaaaaactaacagatatcaaaagatatctctaactcagtccattgtgttcagttcaatcatgagctctctcaagaaaattttgggtattaaagggaaagggaagaaatctaagaaattaggtatggctcccccaccctatgaagaagagactccaatggaatattctccaagtgcaccttatgataagtcattgtttggagtcgaagatatggatttccatgatcaacgtcaactccgatatgagaaatttcacttctcattgaagatgactgtgagatcaaacaaaccatttcgaaattatgatgacgttgcagcagcggtgtccaattgggatcatatgtacatcggcatggcaggaaaacgtcctttttataagatattagcattcatgggttctactctattgaaggctacaccagccgtctgggctgaccaaggacagccagaatatcatgctcactgtgagggacgagcttacttgccgcatcggttagggccgacccctccgatgttgaatgtccctgaacattttcgccgtccatttaacatcggattattcagagggacaatcgacataaccctggtacttttcgatgatgaatctgtagattctgccccggtcatatgggatcattttaatgcatccagattgagcagcttcagagaaaaggctttgttgtttggtttgattctagaaaagaaagccactgggaattgggtattggactctattagtcatttcaagtaattatcacaagtgttgaggtgatgggcagactatgaaaaaaactaacagggttcaaacactcttgatcgaggtacccagttatatttgttacaacaatgttgagactttttctcttttgtttcttggccttaggagcccactccaaatttactatagtattccctcatcatcaaaaagggaattggaagaatgtgccttccacatatcattattgcccttctagttctgaccagaattggcataatgatttgactggagttagtcttcatgtgaaaattcccaaaagtcacaaagctatacaagcagatggctggatgtgccacgctgctaaatgggtgactacttgtgacttcagatggtacggacccaaatacatcacgcattccatacactctatgtcacccaccctagaacagtgcaagaccagtattgagcagacaaagcaaggagtttggattaatccaggctttccccctcaaagctgcggatatgctacagtgacggatgcagaggtggttgttgtacaagcaacacctcatcatgtgttggttgatgagtacacaggagaatggattgactcacaattggtggggggcaaatgttccaaggaggtttgtcaaacggttcacaactcgaccgtgtggcatgctgattacaagattacagggctgtgcgagtcaaatctggcatcagtggatatcaccttcttctctgaggatggtcaaaagacgtctttgggaaaaccgaacactggattcaggagtaatcactttgcttacgaaagtggagagaaggcatgccgtatgcagtactgcacacgatggggaatccgactaccttctggagtatggtttgaattagtggacaaagatctcttccaggcggcaaaattgcctgaatgtcctagaggatccagtatctcagctccttctcagacttctgtggatgttagtttgatacaagacgtagagaggatcttagattactctctatgccaggagacgtggagtaagatacgagccaagcttcctgtatctccagtagatctgagttatctcgccccaaaaaatccagggagcggaccggccttcactatcattaatggcactttgaaatatttcgaaacaagatacatcagagttgacataagtaatcccatcatccctcacatggtgggaacaatgagtggaaccacgactgagcgtgaattgtggaatgattggtatccatatgaagacgtagagattggtccaaatggggtgttgaaaactcccactggtttcaagtttccgctgtacatgattgggcacggaatgttggattccgatctccacaaatcctcccaggctcaagtcttcgaacatccacacgcaaaggacgctgcatcacagcttcctgatgatgagactttattttttggtgacacaggactatcaaaaaacccagtagagttagtagaaggctggttcagtagctggaagagcacattggcatcgttctttctgattataggcttgggggttgcattaatcttcatcattcgaattattgttgcgattcgctataaatacaaggggaggaagacccaaaaaatttacaatgatgtcgagatgagtcgattgggaaataaataacagatgacgcatgagggtcagatcagatttacagcgtaagtgtgatatttaggattataaaggttccttaattttaatttgttacgcgttgtatgaaaaaaactcatcaacagccatcatggaatcacggaaggacatcactaatcaggaggaactgtggaaaatgaagccaagaaggaatctggaagaggacgactatctgcacaaggacaccggcgaaacaagtatgctgaaacgaccagtgctgctgcacctgcatcagactgctcacgcagacgagtttgattgcccctctgaactgcagcacacccaggagctgttcccacagtggcatctgcccatcaagattgccgctatcattgcttcactgacatttctgtacactctgctgagagaagtgatccaccccctggccaccagccatcagcagtacttctataagatccctatcctggtcatcaacaaggtcctgccaatggtgagcatcacactgctggccctggtctacctgcctggagtgatcgcagccattgtccagctgcacaatgggacaaagtataagaaatttccacattggctggataagtggatgctgactaggaaacagttcggactgctgtccttctttttcgccgtgctgcacgctatctacagcctgtcctatcccatgaggaggagctaccggtataagctgctgaactgggcttaccagcaggtgcagcagaacaaggaggacgcatggattgaacatgacgtgtggcgcatggaaatctacgtgagcctgggcattgtcggactggccatcctggctctgctggcagtgaccagtatcccttctgtcagtgactcactgacatggagagagtttcactacattcagagcaagctggggatcgtgtccctgctgctgggcaccatccatgcactgatttttgcctggaacaagtggatcgatatcaagcagttcgtgtggtatactccccctacctttatgattgccgtcttcctgcccatcgtggtcctgatcttcaagtccatcctgttcctgccttgtctgcggaagaaaatcctgaaaattcggcacggatgggaggatgtcaccaaaatcaataagactgaaatctgtagccagctgaagctttaacgtacgtgtatgaaaaaaactcatcaacagccatcatggatgttaacgattttgagttgcatgaggactttgcattgtctgaagatgactttgtcacttcagaatttctcaatccggaagaccaaatgacatacctgaatcatgccgattataatttgaattctcccttaatcagcgatgatattgatttcctgatcaagaaatataatcatgagcaaattccgaaaatgtgggatgtcaagaattgggagggagtgttagagatgttgacagcctgtcaagccagtccaattttatctagcactatgcataagtgggtgggaaagtggctcatgtctgatgatcatgacgcaagccaaggcttcagttttcttcatgaagtggacaaagaagctgatctgacgtttgaggtggtggagacattcattagaggatggggaggtcgagaattgcagtacaagaggaaagacacatttccggactcctttagagttgcagcctcattgtgtcaaaaattccttgatttgcacaaactcactctgataatgaattcagtctctgaagtcgaacttaccaacctagcaaagaattttaaaggaaaaaacaggaaagcaaaaagcggaaatctgataaccagattgagggttcccagtttaggtcctgcttttgtgactcagggatgggtgtacatgaagaagttggaaatgattatggatcggaattttttgttgatgttgaaagacgttatcatcgggaggatgcagacgatcctgtccatgatctcaagagatgataatctcttctccgagtctgatatctttactgtattaaagatataccggataggggataagatattagaaaggcaagggacaaagggttacgacttgatcaaaatgattgagcctatttgtaacttaaagatgatgaatctggcacgtaaatatcgtcctctcatccctacatttcctcattttgaaaaacatattgctgactctgttaaggaaggatcgaaaatagacaaagggattgagtttatatatgatcacattatgtcaatccctggtgtggacttgaccttagttatttacggatcatttcggcactggggtcatccttttatcaactactatgagggcttagagaagctacacaagcaggttacaatgcccaagactattgacagagaatatgcagaatgtcttgctagtgatctggcaagaatcgttcttcagcaacaattcaatgaacataagaaatggtttgttgatgtagataaagtcccacaatcccatcctttcaaaagccatatgaaagagaatacttggcctactgcagcccaagttcaggattacggcgatcgctggcatcagctcccactcatcaaatgcttcgaaatcccagatttgttagatccatcgatcatctactcagacaaaagtcattccatgaaccggtctgaagtactacgacatgtaagacttacacctcatgtgcccattccaagcaggaaagtattgcagacaatgttggagactaaggcaacagactggaaagagtttttaaagaaaattgacgaagaggggttagaggatgatgatcttgtcataggactcaaagggaaagagagagaattaaaaattgcgggaagattcttttctttgatgtcctggaagctcagagagtattttgtcatcactgagtatttgattaagacgcactttgtcccgatgtttaaagggttgaccatggcggatgacttgacagcggtgataaagaagatgatggacacatcttcaggacaaggcttagataattatgaatccatttgtatagccaaccatattgactatgagaagtggaacaatcatcaaagaaaagagtcgaacgggcccgtgttcaaggtgatgggtcaattcttgggatatccacgtctgattgagagaactcatgaattttttgagaagagtctgatatattacaatggacgaccagatctgatgcgggttcgaggaaattctctagtcaacgcctcatctttaaatgtctgctgggagggtcaagctgggggattagaaggactgcgacagaagggatggagtattctaaatttgcttgtcattcagagagaagcaaaaataaggaacaccgccgtgaaagtgctagctcaaggtgacaatcaggtgatatgtactcagtataaaacgaagaaatcccggaatgatattgagcttaaggcagctctaacacagatggtatctaataatgagatgattatgtctgcgattaaatcaggcaccgagaaactgggtcttttgattaatgatgatgagacaatgcaatctgctgattacctcaattacgggaaggttcccattttcagaggagtaatcagaggccttgagacaaaaagatggtctcgagtgacctgtgtgacaaatgatcagattccaacgtgtgcgaacattatgagctctgtgtcaactaatgcattaactgtagcccattttgccgagaatccagtcaatgccatcattcagtataactactttggaacatttgcaaggctactgctgatgatgcatgaccccgctctgaggatctctctgtatgaagtccaatcaaaaattccaggacttcacagtttgacatttaaatattctatgttgtatctggatccttcgataggaggagtctccggaatgtcactctcgagattcctcataagatcatttccagatccagtgacagaaagtttggcgttctggaaatttatccactctcatgcaagaagcgattcattaaaggagatatgtgcagtttttggaaatcctgaaattgcaagatttcggctaactcatgtcgataaattggtggaagacccaacctcattgaacatagctatgggaatgagtcctgctaatctattaaagacagaggtaaaaaaatgtctactggaatcaaggcagagcatcaagaaccagattgtaagagatgctactatttacctacaccatgaggaagacaaacttcgtagtttcttatggtccataacaccactgttccctcggttcttgagtgaattcaaatctgggacattcatcggagtagcagatggcctgatcagcttatttcagaactctaggactattcgaaattcttttaaaaagcgttatcacagggaacttgatgatttaataatcaagagcgaagtttcctcacttatgcatttgggtaagctacatttgaggcgaggctcagttcgtatgtggacttgctcttctactcaggctgatcttctccgattccggtcatggggaagatctgttataggaaccacagtccctcatcccttagagatgttaggacaacattttaaaaaggagactccttgcagtgcttgcaacatatccggattagactatgtatctgtccactgtccgaatgggattcatgacgtttttgaatcacgtggtccactccctgcatatttgggttctaaaacatccgaatcaacttcgatcttgcagccgtgggagagagagagtaaagtaccgttgattaagcgtgccacaaggcttcgtgatgcaatttcatggtttgtgtctcccgactctaacttggcctcaactatccttaagaacataaatgcattaacaggagaagaatggtcaaagaagcagcatggatttaaaaggacgggatcggcgttacacaggttctccacatccaggatgagtcatggtggttttgcttctcagagtacggctgccttgactagattgatggcaactactgacactatgagagatctgggagaacagaactatgatttcctgtttcaggcgacattattgtatgctcaaataaccacaactgtagtcaggaatggatcatttcatagctgcacggaccattaccatataacctgcaaatcttgtctgagggccattgatgagattaccttggattcagcgatggaatatagccctccagatgtatcatcagttttacaatcttggaggaatggagaaggctcttggggacatgaagtgaaacaaatatacccagttgaaggtgactggaggggactatctcctgttgaacaatcttatcaagtcggacgctgtatcgggtttctgttcggtgatctggcgtatagaaaatcatcccatgcagatgatagctccatgtttccgttatctatacaaaacaaagtcagaggaagaggctttttaaaagggcttatggatgggttaatgagagccagttgttgccaggtgatccatcgtcgaagcttagcccatctgaagagaccggctaatgcagtctatggagggctgatttatttgatagacaaattgagtgcatctgccccttttctttcactgacgagacatggacctttaagggaagaattagaaactgttccacataagataccgacttcttatcctacgagcaaccgagatatgggggtgatagttcgtaattattttaaatatcagtgcagactggtagaaaaaggtcggtacaagacacattatcctcaattgtggcttttctcagatgtgctgtccattgatttcttaggacccctgtctatatcttcaactctattgggtattctgtataaacagacgttatcttctcgagacaaaaatgagttgagagaactcgctaacttgtcttcattgttgagatcaggagaaggatgggaagatatccatgtcaaattcttctctaaggacactttactctgccctgaagagatccgacatgcgtgcaaatttgggattgctaaggaatccgctgttttaagctattatcctccttggtctcaagagtcttatggaggcatcacctcgatccccgtatatttttcgaccaggaagtatcccaaaattttagatgtccctcctcgggttcaaaacccattggtctcgggtctacgattggggcaactccctactggagcacattataagattaggagcattgtaaagaacaagaaccttcgttatagagatttccttagttgtggggatggatctggggggatgaccgcggcactattgagagaaaacagacaaagtaggggaatcttcaacagcctgttagagttagccggatctcttatgagaggagcatctccagagcctccaagtgcactggagacgctcgggcaagaacgatctaggtgtgtgaatggaagcacatgttgggagtactcatctgacctaagccaaaaagagacatgggattacttcttaagattgaagagaggcctgggtttgaccgtggacttaatcaccatggacatggaggtcagagaccctaatacaagtttgatgatagaaaagaacctcaaagtttatctgcatcagatattagaaccaactggtgtcttaatatataaaacatacgggacccatattgcgacacaaacagataatatcctgacgataatcggtcctttctttgagacggttgacctagtccagtccgaatacagcagctcacaaacgtccgaggtctattttgtaggacgaggcttgcgctctcatgttgacgaaccctgggtggactggccatccttaatggacaattggagatccatttatgcttttcatgatcctactacagaatttatcagagcaaaaaaagtctgtgaaattgacagtcttataggcattccggctcaattcattccagacccatttgtaaatctcgagaccatgctacagatagttggtgttccaacaggagtttcgcatgccgcagctctattatcatcacaatatccaaatcaattggtcacaacgtcaatattttatatgacactcgtgtcttattataatgtaaaccatattcgaagaagccccaagcctttctctcctccgtctgatggagtctcacagaacattggttcagccatagtcggactaagtttttgggtgagtttgatggagaatgatctcggattatacaaacaggctctaggtgcaataaagacgtcattccctattagatggtcctctgtccagaccaaggatgggtttacacaagaatggagaactaaaggaaacggaattcctaaagattgtcgtctctcagactctttggctcagataggaaactggatcagagcgatggaattggttaggaacaaaacgaggcaatcaggattttctgaaaccctatttgatcaattctgcggacttgcagaccatcacctcaaatggcggaagttgggaaacagaacaggaattattgattggctaaataatagaatttcatccattgacaaatccatcttggtgaccaaaagtgatctgcatgacgagaactcatggagggagtgaagatgtattcttccacctctcattgggtgatacccatatatgaaaaaaactataagtactttaaactctctttgttttttaatgtatatctggttttgttgtttccgt

SEQ ID NO: 16 - protein, 10 aa, Artificial Sequence
  synthetic H-2Kb binding E6 peptide
EVYDFAFRDL

SEQ ID NO: 17 - protein, 9 aa, Artificial Sequence
  synthetic H-2Db binding E7 peptide
RAHYNIVTF

SEQ ID NO: 18 - DNA, 30 bp, Artificial Sequence
  synthetic primer
actggaattcatgcatcagaagcgaactgc

SEQ ID NO: 19 - DNA, 29 bp, Artificial Sequence
  synthetic primer
actgggatcctcactgctgggaggcacac

SEQ ID NO: 20 - protein, 8 aa, Artificial Sequence
  synthetic
RSYRYKLL

SEQ ID NO: 21 - protein, 9 aa, Artificial Sequence
  synthetic
VTKINKTEI

SEQ ID NO: 22 - protein, 9 aa, Artificial Sequence
  synthetic
VSKINRTEM

SEQ ID NO: 23 - protein, 9 aa, Artificial Sequence
  synthetic
KDITNQEEL